(19) United States
(12) Patent Application Publication
(10) Pub. No.: US 2008/0044426 A1
(43) Pub. Date: Feb. 21, 2008

(54) Novel Atypical Pneumonia-Causing Virus

(76) Inventors: Jan Cornelis De Jong, Eh Gouda (NL); Theodorus Marinus Bestebroer, Ve Krimpen Aanden Ijssel (NL); James Henry Matthew Simon, Amsterdam (NL); Ronaldus Adrianus Maria Fouchier, Rotterdam (NL); Albertus Dominicus Marcellinus Osterhaus, Antwerpen-Zuid (BE)

Correspondence Address: HOFFMANN & BARON, LLP, 6900 JERICHO TURNPIKE, SYOSSET, NY, 11791, US

(21) Appl. No.: 10/579,614

(22) PCT Filed: Nov. 18, 2004
(86) PCT No.: PCT/NL04/00805

§ 371 Date: Jul. 19, 2007

(30) Foreign Application Priority Data
Nov. 18, 2003 (EP) 03078613.1
Dec. 1, 2003 (EP) 03078772.5

(51) Int. Cl. A61K 39/215; A61K 39/395; A61K 48/00; C07K 16/10; C12N 1/21; C12N 15/40; C12N 15/63; C12N 5/10; C12N 7/01; C12N 9/12; C12Q 1/70

(52) U.S. Cl. 424/159.1; 424/221.1; 435/194; 435/235.1; 435/252.3; 435/348; 435/5; 514/2; 514/44; 530/350; 530/389.4; 536/23.72

(57) ABSTRACT

The invention relates to the field of virology. The invention provides a new isolated essentially mammalian positive-sense single stranded RNA virus (EMCR-CoV) within the group of coronaviuses and components thereof.

The invention relates to the field of virology.

Recently, a respiratory illness (atypical pneumonia) was diagnosed in an 8 months old patient that could not be attributed to SARS (Severe Acute Respiratory Syndrome) virus or any other known viral infection. The patient tested negative for influenza, parainfluenza, mumps and RSV and yet the disease was identified to be caused by a virus which closely resembled SARS.

For being able to trace its origin, monitor its epidemiology and prevent possible spreading of the disease, it is of great importance to be able to recognise viral causes of pneumonia in an early stage. Especially, if severe diseases are found to be caused by viruses, it is necessary to detect the identity of the virus as soon as possible, in order to develop diagnostic tools and possibly therapies. The SARS epidemy has shown that it is paramount for prevention of spread of the disease to be able to get an early diagnosis in order to timely take effective isolation measures en initiate quarantaine precautions. Only then, world-wide contaminations can be prevented.

Furthermore, identification of the viral cause for the disease enables development of vaccines, which can be used prophylactically to protect people who are at risk of being infected. And, finally, knowledge of the viral cause enables to develop therapeutic measures.

Thus, there is great need in developing diagnostic tools and therapies for viral pneumonias in general, and particular to a novel disease-causing infectious agent, especially when this agent appears to be a virus.

The invention provides the nucleotide sequence of an isolated essentially mammalian positive-sense single stranded RNA virus belonging to the Coronaviruses, which is the causative factor for the new disease, hereinafter referred to as EMCR-CoV and the disease being referred to as EMCR-CoV-caused pneumonia. From a phylogenetic analysis of the Matrix and Nucleocapsid gene sequences of the virus (FIGS. 2a and 2b) it appears that the virus is a distinct member of the group formed by PEDV (porcine epidemic diarrhea virus), HCoV-229E (human coronavirus 229E), PRCoV (porcine respiratory coronavirus), TGEV (transmissible gastroenteritis virus), CaCoV (Canine coronavirus) and FeCoV (feline coronavirus). Based on amino acid identity matrices, human coronavirus 229E seems to be the closest relative (for all ORFs with the exception of Matrix which appears to be slightly more closely related to PEDV—see FIG. 3).

Although phylogenetic analyses provide a convenient method of identifying a virus, several other possibly more straightforward albeit somewhat more coarse methods for identifying said virus or viral proteins or nucleic acids from said virus are herein also provided. As a rule of thumb an EMCR-Coronavirus can be identified by the percentages of homology of the virus, proteins or nucleic acids to be identified in comparison with viral proteins or nucleic acids identified herein by sequence. It is generally known that virus species, especially RNA virus species, often constitute a quasi species wherein a cluster of said viruses displays heterogeneity among its members. Thus it is expected that each isolate may have a somewhat different percentage relationship with the sequences of the isolate as provided herein.

When one wishes to compare a virus isolate with the sequences as listed in FIG. 1 molo, the invention provides an isolated essentially mammalian positive-sense single stranded RNA virus (EMCR-CoV) belonging to the Coronaviruses and identifiable as phylogenetically corresponding thereto by determining a nucleic acid sequence of said virus and determining that said nucleic acid sequence has a percentage nucleic acid identity to the sequences as listed higher than the percentages identified herein for the nucleic acids as identified herein below in comparison with PEDV, 229E, PRCoV, TGEV, CaCoV and FeCoV. Likewise, an isolated essentially mammalian positive-sense single stranded RNA virus (EMCR-CoV) belonging to the Coronaviruses and identifiable as phylogenetically corresponding thereto by determining an amino acid sequence of said virus and determining that said amino acid sequence has a percentage amino acid homology to the sequences as listed which is essentially higher than the percentages provided herein in comparison with PEDV, 229E, PRCoV, TGEV, CaCoV and FeCoV.

With the provision of the sequence information of this EMCR-Coronavirus (EMCR-CoV), the invention provides diagnostic means and methods, prophylactic means and methods and therapeutic means and methods to be employed in the diagnosis, prevention and/or treatment of disease, in particular of respiratory disease (atypical pneumonia), in particular of mammals, more in particular in humans associated with infection by this virus. In virology, it is most advisory that diagnosis, prophylaxis and/or treatment of a specific viral infection is performed with reagents that are most specific for said specific virus causing said infection. In this case this means that it is preferred that said diagnosis, prophylaxis and/or treatment of an EMCR-CoV virus infection is performed with reagents that are most specific for EMCR-CoV virus. This by no means however excludes the possibility that less specific, but sufficiently cross-reactive reagents are used instead, for example because they are more easily available and sufficiently address the task at hand.

The invention for example provides a method for virologically diagnosing an EMCR-CoV infection of an animal, in particular of a mammal, more in particular of a human being, comprising determining in a sample of said animal the presence of a viral isolate or component thereof by reacting said sample with an EMCR-CoV specific nucleic acid or antibody according to the invention, and a method for serologically diagnosing an EMCR-CoV infection of a mammal comprising determining in a sample of said mammal the presence of an antibody specifically directed against an EMCR-CoV virus or component thereof by reacting said sample with an EMCR-CoV virus-specific proteinaceous molecule or fragment thereof or an antigen according to the invention.

The invention also provides a diagnostic kit for diagnosing an EMCR-CoV infection comprising an EMCR-CoV virus, an EMCR-CoV virus-specific nucleic acid, proteinaceous molecule or fragment thereof, antigen and/or an antibody according to the invention, and preferably a means for detecting said EMCR-CoV virus, EMCR-CoV virus-specific nucleic acid, proteinaceous molecule or fragment thereof, antigen and/or an antibody, said means for example comprising an excitable group such as a fluorophore or enzymatic detection system used in the art (examples of suitable diagnostic kit format comprise IF, ELISA, neutralization assay, RT-PCR assay). To determine whether an as yet unidentified virus component or synthetic analogue thereof such as nucleic acid, proteinaceous molecule or fragment thereof can be identified as EMCR-CoV-virus-specific, it suffices to analyse the nucleic acid or amino acid sequence of said component, for example for a stretch of said nucleic acid or amino acid, preferably of at least 10, more preferably at least 25, more preferably at least 40 nucleotides or amino acids (respectively), by sequence homology comparison with the provided EMCR-CoV viral sequences and with known non-EMCR-CoV viral sequences (human coronavirus 299E is preferably used) using for example phylogenetic analyses as provided herein. Depending on the degree of relationship with said EMCR-CoV or non-EMCR-CoV viral sequences, the component or synthetic analogue can be identified.

The invention thus provides the nucleotide sequence of a novel etiological agent, an isolated essentially mammalian positive-sense single stranded RNA virus (herein also called EMCR-CoV virus) belonging to the Coronaviridae family, and EMCR-CoV virus-specific components or synthetic analogues thereof.

Coronaviruses were first isolated from chickens in 1937, while the first human coronavirus was propagated in vitro by Tyrell and Bonoe in 1965. There are now about 13 species in this family, which infect cattle, pigs, rodents, cats, dogs, birds and man. Coronavirus particles are irregularly shaped, about 60-220 nm in diameter, with an outer envelope bearing distinctive, ‘club-shaped’ peplomers (about 20 nm long and 10 nm wide at the distal end). This ‘crown-like’ appearance give the family its name. The envelope carries two glycoproteins: S, the spike glycoprotein which is involved in cell fusion and is a major antigen, and M, the membrane glycoprotein, which is involved in budding and envelope formation. The genome is associated with a basic phosphoprotein, designated N. The genome of coronaviruses, a single stranded positive-sense RNA strand, is typically 27-31 Kb long and contains a 5′ methylated cap and a 3′ poly-A tail, by which it can directly function as an mRNA in the infected cell. Initially the 5′ ORF 1 (about 20 Kb) is translated to produce a viral polymerase, which then produces a full length negative sense strand. This is used as a template to produce mRNA as a ‘nested set’ of transcripts, all with identical 5′ non-translated leader sequence of 72 nucleotides and coincident 3′ polyadenylated ends. Each mRNA thus produced is monocistronic, the genes at the 5′ end being translated from the longest mRNA and so on. These unusual cytoplasmic structures are produced not by splicing, but by the polymerase during transcription. Between each of the genes there is a repeated intergenic sequence—AACUAAAC—which interacts with the transcriptase plus cellular factors to splice the leader sequence onto the start of each ORF. In some coronaviruses there are about 8 ORFs, coding for the proteins mentioned above, but also for a heamagglutenin esterase (HE), and several other non-structural proteins.

Newly isolated viruses are phylogenetically corresponding to and thus taxonomically corresponding to EMCR-CoV virus when comprising a gene order and/or amino acid sequence and/or nucleotide sequence sufficiently similar to our prototypic EMCR-CoV virus. The highest amino acid sequence identity, between ORFs of EMCR-CoV virus and any of the known other viruses of the same family to date are with human coronavirus 299E or Porcine Epidemic Diarrhea Virus (see FIGS. 3 and 4). The amino acid identities with human coronavirus 229E ranges from 45% (Nucleoprotein) to 81% (Replicase 1b); interestingly, Replicase 1a has an identity of just 56% contrasting with Replicase 1b's 81% identity. EMCR CoV has a closer identity with human coronavirus 229E than with any of the known other viruses of the same family to date for all putative ORFs, with the exception of Matrix, which is slightly more closely related to the Matrix ORF of PEDV. Individual proteins or whole virus isolates with, respectively, higher homology than these mentioned maximum values are considered phylogenetically corresponding and thus taxonomically corresponding to EMCR-CoV virus, and generally will be encoded by a nucleic acid sequence structurally corresponding with a sequence as shown in FIG. 1. Herewith the invention provides a virus phylogenetically corresponding to the isolated virus of which the sequences are depicted in FIG. 1.

It should be noted that, similar to other viruses, a certain degree of variation can be expected to be found between EMCR-CoV-viruses isolated from different sources.

Also, the viral sequence of the EMCR-CoV virus or an isolated EMCR-CoV virus gene as provided herein for example shows less than 95%, preferably less than 90%, more preferably less than 80%, more preferably less than 70% and most preferably less than 65% nucleotide sequence homology or less than 95%, preferably less than 90%, more preferably less than 80%, more preferably less than 70% and most preferably less than 65% amino acid sequence homology with the respective nucleotide or amino acid sequence of the human coronavirus 299E or Porcine Epidemic Diarrhea Virus as for example can be found in Genbank (for example in accession number af304460 (HCoV-299E) or af353511 (PEDV).

Sequence divergence of EMCR-CoV strains around the world may be somewhat higher, in analogy with other coronaviruses.

The term “nucleotide sequence homology” as used herein denotes the presence of homology between two (poly)nucleotides. Polynucleotides have “homologous” sequences if the sequence of nucleotides in the two sequences is the same when aligned for maximum correspondence. Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence homology” for polynucleotides, such as 50, 60, 70, 80, 90, 95, 98, 99 or 100 percent sequence homology may be determined by comparing two optimally aligned sequences over a comparison window, wherein the portion of the polynucleotide sequence in the comparison window may include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology. Optimal alignment of sequences for comparison may be conducted by computerized implementations of known algorithms, or by inspection. Readily available sequence comparison and multiple sequence alignment algorithms are, respectively, the Basic Local Alignment Search Tool (BLAST) (Altschul, S. F. et al.1990. J. Mol. Biol. 215:403; Altschul, S. F. et al. 1997. Nucleic Acid Res. 25:3389-3402) and ClustalW programs both available on the internet. Other suitable programs include GAP, BESTFIT and FASTA in the Wisconsin Genetics Software Package (Genetics Computer Group (GCG), Madison, Wis., USA).

As used herein, “substantially complementary” means that two nucleic acid sequences have at least about 65%, preferably about 70%, more preferably about 80%, even more preferably 90%, and most preferably about 98%, sequence complementarity to each other. This means that the primers and probes must exhibit sufficient complementarity to their template and target nucleic acid, respectively, to hybridise under stringent conditions. Therefore, the primer sequences as disclosed in this specification need not reflect the exact sequence of the binding region on the template and degenerate primers can be used. A substantially complementary primer sequence is one that has sufficient sequence complementarity to the amplification template to result in primer binding and second-strand synthesis.

The term “hybrid” refers to a double-stranded nucleic acid molecule, or duplex, formed by hydrogen bonding between complementary nucleotides. The terms “hybridise” or “anneal” refer to the process by which single strands of nucleic acid sequences form double-helical segments through hydrogen bonding between complementary nucleotides.

The term “oligonucleotide” refers to a short sequence of nucleotide monomers (usually 6 to 100 nucleotides) joined by phosphorous linkages (e.g., phosphodiester, alkyl and aryl-phosphate, phosphorothioate), or non-phosphorous linkages (e.g., peptide, sulfamate and others). An oligonucleotide may contain modified nucleotides having modified bases (e.g., 5-methyl cytosine) and modified sugar groups (e.g., 2′-O-methyl ribosyl, 2′-O-methoxyethyl ribosyl, 2′-fluoro ribosyl, 2′-amino ribosyl, and the like). Oligonucleotides may be naturally-occurring or synthetic molecules of double- and single-stranded DNA and double- and single-stranded RNA with circular, branched or linear shapes and optionally including domains capable of forming stable secondary structures (e.g., stem-and-loop and loop-stem-loop structures).

The term “primer” as used herein refers to an oligonucleotide which is capable of annealing to the amplification target allowing a DNA polymerase to attach thereby serving as a point of initiation of DNA synthesis when placed under conditions in which synthesis of primer extension product which is complementary to a nucleic acid strand is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxy ribonucleotide. The primer must be sufficiently long to prime the synthesis of extension products in the presence of the agent for polymerization. The exact lengths of the primers will depend on many factors, including temperature and source of primer. A “pair of bidirectional primers” as used herein refers to one forward and one reverse primer as commonly used in the art of DNA amplification such as in PCR amplification.

The term “probe” refers to a single-stranded oligonucleotide sequence that will recognize and form a hydrogen-bonded duplex with a complementary sequence in a target nucleic acid sequence analyte or its cDNA derivative.

The terms “stringency” or “stringent hybridization conditions” refer to hybridization conditions that affect the stability of hybrids, e.g., temperature, salt concentration, pH, formamide concentration and the like. These conditions are empirically optimised to maximize specific binding and minimize non-specific binding of primer or probe to its target nucleic acid sequence. The terms as used include reference to conditions under which a probe or primer will hybridise to its target sequence, to a detectably greater degree than other sequences (e.g. at least 2-fold over background). Stringent conditions are sequence dependent and will be different in different circumstances. Longer sequences hybridise specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridises to a perfectly matched probe or primer. Typically, stringent conditions will be those in which the salt concentration is less than about 1.0 M Na+ion, typically about 0.01 to 1.0 M Na+ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1 M NaCl, 1% SDS at 37° C., and a wash in 0.1×SSC at 60° C. Hybridization procedures are well known in the art and are described in e.g. Ausubel et al, Current Protocols in Molecular Biology, John Wiley & Sons Inc., 1994.

The term “antibody” includes reference to antigen binding forms of antibodies (e.g., Fab, F (ab) 2). The term “antibody” frequently refers to a polypeptide substantially encoded by an immunoglobulin gene or immunoglobulin genes, or fragments thereof which specifically bind and recognize an analyte (antigen). However, while various antibody fragments can be defined in terms of the digestion of an intact antibody, one of skill will appreciate that such fragments may be synthesized de novo either chemically or by utilizing recombinant DNA methodology. Thus, the term antibody, as used herein, also includes antibody fragments such as single chain Fv, chimeric antibodies (i. e., comprising constant and variable regions from different species), humanized antibodies (i. e., comprising a complementarity determining region (CDR) from a non-human source) and heteroconjugate antibodies (e. g., bispecific antibodies).

In short, the invention provides an isolated essentially mammalian positive-sense single stranded RNA virus (EMCR-CoV) belonging to the Coronaviruses and identifiable as phylogenetically corresponding thereto by determining a nucleic acid sequence of a suitable fragment of the genome of said virus and testing it in phylogenetic tree analyses wherein maximum likelihood trees are generated using 100 bootstraps and 3 jumbles and finding it to be more closely phylogenetically corresponding to a virus isolate having the sequences as depicted in FIG. 1 than it is corresponding to a virus isolate of PEDV (porcine epidemic diarrhea virus), HCoV-229E (human coronavirus 229E), PRCoV (porcine respiratory coronavirus), TGEV (transmissible gastroenteritis virus), CaCoV (Canine coronavirus) and FeCoV (feline coronavirus).

Suitable nucleic acid genome fragments each useful for such phylogenetic tree analyses are for example any of the fragments encoding the Matrix protein or the Nucleocapsid protein as disclosed in FIG. 1, leading to the phylogenetic tree analysis as disclosed herein in FIG. 2a or 2b. Other suitable nucleic acid fragments useful for such phylogenetic tree analyses are for example any of the fragments encoding Replicase 1a and 1b, Spike, orf 4a and 4b, and E.

A suitable open reading frame (ORF) useful in phylogenetic analyses comprises the ORF encoding the viral replicase (ORF 1a). When an overall amino acid identity of at least 60%, preferably of at least 70%, more preferably of at least 80%, more preferably of at least 90%, most preferably of at least 95% of the analysed replicase with the replicase having a sequence comprising the amino acids of FIG. 1 is found, the analysed virus isolate comprises an EMCR-CoV virus isolate according to the invention.

A suitable open reading frame (ORF) useful in phylogenetic analyses comprises the ORF encoding the viral replicase (ORF 1b). When an overall amino acid identity of at least 82%, more preferably of at least 90%, most preferably of at least 95% of the analysed replicase with the replicase having a sequence comprising the amino acids of FIG. 1 is found, the analysed virus isolate comprises an EMCR-CoV virus isolate according to the invention.

Another suitable open reading frame (ORF) useful in phylogenetic analyses comprises the ORF encoding the Nucleocapsid protein. When an overall amino acid identity of at least 50%, more preferably of at least 60%, more preferably of at least 70%, more preferably of at least 80%, more preferably of at least 90%, most preferably of at least 95% of the analysed Nucleocapsid protein with the Nucleocapsid protein encoded by a sequence comprising (part of) the sequence F of FIG. 1 is found, the analysed virus isolate comprises an EMCR-CoV isolate according to the invention.

Another suitable open reading frame (ORF) useful in phylogenetic analyses comprises the ORF encoding the Matrix protein. When an overall amino acid identity of at least 60%, more preferably of at least 70%, more preferably of at least 80%, more preferably of at least 90%, most preferably of at least 95% of the analysed Matrix protein with the Matrix protein encoded by a sequence comprising (part of) the sequence F of FIG. 1 is found, the analysed virus isolate comprises an EMCR-CoV isolate according to the invention.

Another suitable open reading frame (ORF) useful in phylogenetic analyses comprises the ORF encoding the spike protein S. When an overall amino acid identity of at least 55%, more preferably of at least 60%, more preferably of at least 70%, more preferably of at least 80%, more preferably of at least 90%, most preferably of at least 95% of the analysed S-protein encoded by a sequence comprising the sequence of translation 2 of E and translation 1 of the F sequence of the S-protein as depicted in FIG. 1 is found, the analysed virus isolate comprises an EMCR-CoV virus isolate according to the invention. The S ORF of the EMCR-CoV virus seems to be located adjacent to the ORF lab (coding for the viral replicase), which would discriminate an EMCR-CoV viruses from the bovine coronavirus and the murine hepatitis virus, which have a so-called 2a gene and an HE-gene between the S protein and the viral polymerase.

The invention provides among others an isolated or recombinant nucleic acid or virus-specific functional fragment thereof obtainable from a virus according to the invention. The isolated or recombinant nucleic acids comprises the sequences as given in FIG. 1 or sequences of homologues which are able to hybridise with those under stringent conditions. In particular, the invention provides primers and/or probes suitable for identifying an EMCR-CoV virus nucleic acid.

Furthermore, the invention provides a vector comprising a nucleic acid according to the invention. To begin with, vectors such as plasmid vectors containing (parts of) the genome of the EMCR-CoV virus, virus vectors containing (parts of) the genome of the EMCR-CoV (for example, but not limited thereto, vaccinia virus, retroviruses, baculovirus), or EMCR-CoV virus containing (parts of) the genome of other viruse or other pathogens are provided.

Also, the invention provides a host cell comprising a nucleic acid or a vector according to the invention. Plasmid or viral vectors containing the replicase components of EMCR-CoV virus are generated in prokaryotic cells for the expression of the components in relevant cell types (bacteria, insect cells, eukaryotic cells). Plasmid or viral vectors containing full-length or partial copies of the EMCR-CoV virus genome will be generated in prokaryotic cells for the expression of viral nucleic acids in-vitro or in-vivo. The latter vectors may contain other viral sequences for the generation of chimeric viruses or chimeric virus proteins, may lack parts of the viral genome for the generation of replication defective virus, and may contain mutations, deletions or insertions for the generation of attenuated viruses.

Infectious copies of EMCR-CoV virus (being wild type, attenuated, replication-defective or chimeric) can be produced upon co-expression of the polymerase components according to the state-of-the-art technologies described above.

In addition, eukaryotic cells, transiently or stably expressing one or more full-length or partial EMCR-CoV virus proteins can be used. Such cells can be made by transfection (proteins or nucleic acid vectors), infection (viral vectors) or transduction (viral vectors) and may be useful for complementation of mentioned wild type, attenuated, replication-defective or chimeric viruses.

A chimeric virus may be of particular use for the generation of recombinant vaccines protecting against two or more viruses. For example, it can be envisaged that EMCR-CoV virus vector expressing one or more proteins of a human metapneumovirus or a human metapneumovirus vector expressing one or more proteins of EMCR-CoV virus will protect individuals vaccinated with such vector against both virus infections. Such a specific chimeric virus is particularly useful in the invention because it is suspected that co-infection of, for instance, human metapneumovirus frequently occurs in coronavirus infected patients. Attenuated and replication-defective viruses may be of use for vaccination purposes with live vaccines as has been suggested for other viruses.

In a preferred embodiment, the invention provides a proteinaceous molecule or coronavirus-specific viral protein or functional fragment thereof encoded by a nucleic acid according to the invention. Useful proteinaceous molecules are for example derived from any of the genes or genomic fragments derivable from a virus according to the invention. Such molecules, or antigenic fragments thereof, as provided herein, are for example useful in diagnostic methods or kits and in pharmaceutical compositions such as sub-unit vaccines and inhibitory peptides. Particularly useful are the viral replicase protein, the spike protein, the matrix protein, the nucleocapsid or antigenic fragments thereof for inclusion as antigen or subunit immunogen, but inactivated whole virus can also be used. Particularly useful are also those proteinaceous substances that are encoded by recombinant nucleic acid fragments that are identified for phylogenetic analyses, of course preferred are those that are within the preferred bounds and metes of ORFs useful in phylogenetic analyses, in particular for eliciting EMCR-CoV virus specific antibodies, whether in vivo (e.g. for protective puposes or for providing diagnostic antibodies) or in vitro (e.g. by phage display technology or another technique useful for generating synthetic antibodies).

Also provided herein are antibodies, be it natural polyclonal or monoclonal, or synthetic (e.g. (phage) library-derived binding molecules) antibodies that specifically react with an antigen comprising a proteinaceous molecule or EMCR-CoV virus-specific functional fragment thereof according to the invention. Such antibodies are useful in a method for identifying a viral isolate as an EMCR-CoV virus comprising reacting said viral isolate or a component thereof with an antibody as provided herein. This can for example be achieved by using purified or non-purified EMCR-CoV virus or parts thereof (proteins, peptides) using ELISA, RIA, FACS or similar formats of antigen detection assays (Current Protocols in Immunology). Alternatively, infected cells or cell cultures may be used to identify viral antigens using classical immunofluorescence or immunohistochemical techniques. Specifically useful in this respect are antibodies raised against EMCR-CoV virus proteins which are encoded by a nucleotide sequence comprising one or more of the sequences disclosed in FIG. 1.

Other methods for identifying a viral isolate as an EMCR-CoV virus comprise reacting said viral isolate or a component thereof with a virus specific nucleic acid according to the invention.

In this way the invention provides a viral isolate identifiable with a method according to the invention as a mammalian virus taxonomically corresponding to a positive-sense single stranded RNA virus identifiable as likely belonging to the EMCR-CoV virus genus within the family of Coronaviruses.

The method is useful in a method for virologically diagnosing an EMCR-CoV virus infection of a mammal, said method for example comprising determining in a sample of said mammal the presence of a viral isolate or component thereof by reacting said sample with a nucleic acid or an antibody according to the invention.

Methods of the invention can in principle be performed by using any nucleic acid amplification method, such as the Polymerase Chain Reaction (PCR; Mullis 1987, U.S. Pat. Nos. 4,683,195, 4,683,202, en 4,800,159) or by using amplification reactions such as Ligase Chain Reaction (LCR; Barany 1991, Proc. Natl. Acad. Sci. USA 88:189-193; EP Appl. No., 320,308), Self-Sustained Sequence Replication (3SR; Guatelli et al., 1990, Proc. Natl. Acad. Sci. USA 87:1874-1878), Strand Displacement Amplification (SDA; U.S. Pat. Nos. 5,270,184, en 5,455,166), Transcriptional Amplification System (TAS; Kwoh et al., Proc. Natl. Acad. Sci. USA 86:1173-1177), Q-Beta Replicase (IAzardi et al., 1988, Bio/Technology 6:1197), Rolling Circle Amplification (RCA; U.S. Pat. No. 5,871,921), Nucleic Acid Sequence Based Amplification (NASBA), Cleavase Fragment Length Polymorphism (U.S. Pat. No. 5,719,028), Isothermal and Chimeric Primer-initiated Amplification of Nucleic Acid (ICAN), Ramification-extension Amplification Method (RAM; U.S. Pat. Nos. 5,719,028 and 5,942,391) or other suitable methods for amplification of nucleic acids.

In order to amplify a nucleic acid with a small number of mismatches to one or more of the amplification primers, an amplification reaction may be performed under conditions of reduced stringency (e.g. a PCR amplification using an annealing temperature of 38° C., or the presence of 3.5 mM MgCl2). The person skilled in the art will be able to select conditions of suitable stringency.

The primers herein are selected to be “substantially” complementary (i.e. at least 65%, more preferably at least 80% perfectly complementary) to their target regions present on the different strands of each specific sequence to be amplified. It is possible to use primer sequences containing e.g. inositol residues or ambiguous bases or even primers that contain one or more mismatches when compared to the target sequence. In general, sequences that exhibit at least 65%, more preferably at least 80% homology with the target DNA or RNA oligonucleotide sequences, are considered suitable for use in a method of the present invention. Sequence mismatches are also not critical when using low stringency hybridization conditions.

The detection of the amplification products can in principle be accomplished by any suitable method known in the art. The detection fragments may be directly stained or labelled with radioactive labels, antibodies, luminescent dyes, fluorescent dyes, or enzyme reagents. Direct DNA stains include for example intercalating dyes such as acridine orange, ethidium bromide, ethidium monoazide or Hoechst dyes. Alternatively, the DNA or RNA fragments may be detected by incorporation of labelled dNTP bases into the synthesized fragments. Detection labels which may be associated with nucleotide bases include e.g. fluorescein, cyanine dye or BrdUrd.

When using a probe-based detection system, a suitable detection procedure for use in the present invention may for example comprise an enzyme immunoassay (EIA) format (Jacobs et al., 1997, J. Clin. Microbiol. 35, 791-795). For performing a detection by manner of the EIA procedure, either the forward or the reverse primer used in the amplification reaction may comprise a capturing group, such as a biotin group for immobilization of target DNA PCR amplicons on e.g. a streptavidin coated microtiter plate wells for subsequent EIA detection of target DNA -amplicons (see below). The skilled person will understand that other groups for immobilization of target DNA PCR amplicons in an EIA format may be employed.

Probes useful for the detection of the target DNA as disclosed herein preferably bind only to at least a part of the DNA sequence region as amplified by the DNA amplification procedure. Those of skill in the art can prepare suitable probes for detection based on the nucleotide sequence of the target DNA without undue experimentation as set out herein. Also the complementary nucleotide sequences, whether DNA or RNA or chemically synthesized analogs, of the target DNA may suitably be used as type-specific detection probes in a method of the invention, provided that such a complementary strand is amplified in the amplification reaction employed.

Suitable detection procedures for use herein may for example comprise immobilization of the amplicons and probing the DNA sequences thereof by e.g. southern blotting. Other formats may comprise an EIA format as described above. To facilitate the detection of binding, the specific amplicon detection probes may comprise a label moiety such as a fluorophore, a chromophore, an enzyme or a radio-label, so as to facilitate monitoring of binding of the probes to the reaction product of the amplification reaction. Such labels are well-known to those skilled in the art and include, for example, fluorescein isothiocyanate (FITC), β-galactosidase, horseradish peroxidase, streptavidin, biotin, digoxigenin, 35S or 125I. Other examples will be apparent to those skilled in the art.

Detection may also be performed by a so called reverse line blot (RLB) assay, such as for instance described by Van den Brule et al. (2002, J. Clin. Microbiol. 40, 779-787). For this purpose RLB probes are preferably synthesized with a 5′ amino group for subsequent immobilization on e.g. carboxyl-coated nylon membranes. The advantage of an RLB format is the ease of the system and its speed, thus allowing for high throughput sample processing.

The use of nucleic acid probes for the detection of RNA or DNA fragments is well known in the art. Mostly these procedure comprise the hybridization of the target nucleic acid with the probe followed by post-hybridization washings. Specificity is typically the function of post-hybridization washes, the critical factors being the ionic strength and temperature of the final wash solution. For nucleic acid hybrids, the Tm can be approximated from the equation of Meinkoth and Wahl, Anal. Biochem., 138: 267-284 (1984): Tm=81.5° C.+16.6(log M)+0.41(% GC)−0.61(% form)−500/L; where M is the molarity of monovalent cations, % GC is the percentage of guanosine and cytosine nucleotides in the nucleic acid, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, the hybridization and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistm and Molecular Biology—Hybridization with Nucleic Acid Probes, Part I, Chapter 2 “Overview of principles of hybridization and the strategy of nucleic acid probe assays”, Elsevier. New York (1993); and Current Protocols in Molecular Biology, Chapter 2, Ausubel, et al., Eds., Greene Publishing and Wiley-Interscience, New York (1995).

In another aspect, the invention provides oligonucleotide probes for the generic detection of target RNA or DNA. The detection probes herein are selected to be “substantially” complementary to one of the strands of the double stranded nucleic acids generated by an amplification reaction of the invention. Preferably the probes are substantially complementary to the immobilizable, e.g. biotin labelled, antisense strands of the amplicons generated from the target RNA or DNA.

It is allowable for detection probes of the present invention to contain one or more mismatches to their target sequence. In general, sequences that exhibit at least 65%, more preferably at least 80% homology with the target oligonucleotide sequences are considered suitable for use in a method of the present invention.

Antibodies, both monoclonal and polyclonal, can also be used for detection purpose in the present invention, for example, in immunoassays in which they can be utilized in liquid phase or bound to a solid phase carrier. In addition, the monoclonal antibodies in these immunoassays can be detectably labeled in various ways. A variety of immunoassay formats may be used to select antibodies specifically reactive with a particular protein (or other analyte). For example, solid-phase ELISA immunoassays are routinely used to select monoclonal antibodies specifically immunoreactive with a protein. See Harlow and Lane, Antibodies, A Laboratory Manual, Cold Spring Harbor Publications, New York (1988), for a description of immunoassay formats and conditions that can be used to determine selective binding. Examples of types of immunoassays that can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be done utilizing immunoassays that are run in either the forward, reverse, or simultaneous modes, including immunohistochemical assays on physiological samples. Those of skill in the art will know, or can readily discern, other immunoassay formats without undue experimentation.

Antibodies can be bound to many different carriers and used to detect the presence of the target molecules. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such using routine experimentation.

The invention also provides a method for serologically diagnosing an EMCR-CoV virus infection of a mammal comprising determining in a sample of said mammal the presence of an antibody specifically directed against an EMCR-CoV virus or component thereof by reacting said sample with a proteinaceous molecule or fragment thereof or an antigen according to the invention

Methods and means provided herein are particularly useful in a diagnostic kit for diagnosing an EMCR-CoV virus infection, be it by virological or serological diagnosis. Such kits or assays may for example comprise a virus, a nucleic acid, a proteinaceous molecule or fragment thereof, an antigen and/or an antibody according to the invention.

Use of a virus, a nucleic acid, a proteinaceous molecule or fragment thereof, an antigen and/or an antibody according to the invention is also provided for the production of a pharmaceutical composition, for example for the treatment or prevention of EMCR-CoV virus infections and/or for the treatment or prevention of atypical pneumonia, in particular in humans. Preferably a peptide comprising part of the amino acid sequence of the spike protein as depicted in the relevant translations of FIG. 1, is used for the preparation of a therapeutic or prophylactic peptide. Also preferably, a protein comprising the amino acid sequence of the spike protein as depicted in the relevant translations of FIG. 1, is used for the preparation of a sub-unit vaccine. Furthermore, the nucleocapsid of Coronaviruses, as depicted in the translation of FIG. 1, is known to be particularly useful for eliciting cell-mediated immunity against Coronaviruses and can be used for the preparation of a sub-unit vaccine.

Attenuation of the virus can be achieved by established methods developed for this purpose, including but not limited to the use of related viruses of other species, serial passages through laboratory animals or/and tissue/cell cultures, serial passages through cell cultures at temperatures below 37° C. (cold-adaption), site directed mutagenesis of molecular clones and exchange of genes or gene fragments between related viruses.

A pharmaceutical composition comprising a virus, a nucleic acid, a proteinaceous molecule or fragment thereof, an antigen and/or an antibody according to the invention can for example be used in a method for the treatment or prevention of an EMCR-CoV virus infection and/or a respiratory illness comprising providing an individual with a pharmaceutical composition according to the invention. This is most useful when said individual comprises a human. Antibodies against EMCR-CoV virus proteins, especially against the spike protein of EMCR-CoV virus, preferably against the amino acid sequence as depicted in the translation in FIG. 1, are also useful for prophylactic or therapeutic purposes, as passive vaccines. It is known from other coronaviruses that the spike protein is a very strong antigen and that antibodies against spike protein can be used in prophylactic and therapeutic vaccination.

The invention also provides method to obtain an antiviral agent useful in the treatment of atypical pneumonia comprising establishing a cell culture or experimental animal comprising a virus according to the invention, treating said culture or animal with an candidate antiviral agent, and determining the effect of said agent on said virus or its infection of said culture or animal. An example of such an antiviral agent comprises an EMCR-CoV virus-neutralising antibody, or functional component thereof, as provided herein, but antiviral agents of other nature are obtained as well.

The invention also provides use of an antiviral agent according to the invention for the preparation of a pharmaceutical composition, in particular for the preparation of a pharmaceutical composition for the treatment of atypical pneumonia, especifically when caused by an EMCR-CoV virus infection, and provides a pharmaceutical composition comprising an antiviral agent according to the invention, useful in a method for the treatment or prevention of an EMCR-CoV virus infection or atypical pneumonia, said method comprising providing an individual with such a pharmaceutical composition.

The invention also comprises an animal model usable for testing of prophylactic and/or therapeutic methods and/or preparations. It is hypothesized that apes can be infected with the EMCR-CoV virus, thereby showing clinical symptoms, and more importantly, similar tissue morphology as found in humans suffering from atypical pneumonia caused by the EMCR-CoV virus. Subjecting apes to a prophylactic or therapeutic treatment either before or during infection with the virus will have a good and useful predictionary value for application of such a prophylaxis or therapy in human subjects.

The invention is further explained in the Examples without limiting it thereto.

### FIGURES LEGENDS

FIG. 1: Nucleotide sequences from parts of the EMCR-CoV virus. Also included are the putative amino acid sequences of polypeptides.

FIG. 2: Phylogenetic relationship for the nucleotide sequences of isolate EMCR-CoV with its closest relatives genetically. Phylogenetic trees were generated by maximum likelihood analyses using 100 bootstraps and 3 jumbles. The scale representing the number of nucleotide changes is shown for each tree. FIG. 1a. Maximum likelihood tree of matrix gene nucleotide sequences. Numbers in trees represent bootstrap values. The scale bar roughly reflects 10% nucleotide differences between related sequences. FIG. 1b. Maximum likelihood tree of nucleocapsid gene nucleotide sequences. Numbers in trees represent bootstrap values. The scale bar roughly reflects 10% nucleotide differences between related sequences.

FIG. 3: Similarity matrices indicating amino acid identity for the putative Replicase 1a, Replicase 1b, Replicase 1ab, Spike, Orf E, Matrix and Nucleocapsid proteins 3a-g, respectively), and for the putative Matrix protein and Nucleoprotein (3h and 3i resp.) between the EMCR-CoV virus and closely related coronaviruses. See text for abbreviations.

FIG. 4 Alignments with various coronaviruses: 5′ untranslated region genomic sequence (a); Putative orf 1a amino acid sequence (b); Putative orf 1b amino acid sequence (c); Putative orf 1ab amino acid sequence (d); Putative Spike amino acid sequence (e); Putative orf 4a amino acid sequence (f); Putative orf 4ab amino acid sequence (g); Putative orf E amino acid sequence (h); Putative Matrix amino acid sequence (i); Putative Nucleoprotein amino acid sequence (j); Putative 3′ untranslated genomic sequence (k); See text for abbreviations.

### EXAMPLES

### Specimen Collection

Virus was collected from an 8 month old patient suffering from pneumonia using nasal swabs.

### Virus Isolation and Culture

Throat swabs were dipped into a culture of tMK cells and passaged four times. Virus was then in Vero-118 cells. One litre of virus containing cell culture supernatant was harvested, and the virus was pelleted in an ultracentrifuge and the virus pellet was resuspended 1 ml PBS.

### RNA Isolation

RNA was isolated from the supernatant of infected cell cultures or sucrose gradient fractions using a High Pure RNA Isolation kit according to instructions from the manufacturer (Roche Diagnostics, Almere, The Netherlands).

### Sequencing

Purified RNA was sent to BaseClear holding BV (Leiden, The Netherlands) for sequencing.

### Phylogenetic Analyses

Nucleotide sequences were aligned using Clustal W running under BioEdit version 5.0.9. Maximum likelihood trees were created using the Seqboot and DNA-ML packages of Phylip 5.6 using 100 bootstraps and 3 jumbles. The consensus trees were calculated using the Consense package of phylip 5.6. These consensus trees were used as usertree in DNA-ML to recalculate the branch lengths from the original sequences.

The sequences of EMCR-CoV were compared with those of reference viruses representing each species in the four groups of coronaviruses. These were: human coronavirus 229E (229E), af304460; porcine epidemic diarrhea virus (PEDV) af353511; transmissible gastroenteritis virus (TGEV), aj271965; bovine coronavirus (BoCoV), af220295; murine hepatitis virus (MHV), af201929; avian infectious bronchitis virus (AIBV), m95169, Canine coronavirus (CaCoV), d13096; feline coronavirus (FeCoV), ay204704; porcine respiratory coronavirus (PRCoV), z24675; human coronavirus OC43 (OC43), m76373, 114643, m933990; porcine haemagglutinating encephalomyelitis virus (HEV), ay078417; rat coronavirus (RtCoV) af207551) References for the viruses are the numbers of the NCBI catalog (http://www.ncbi.nlm.nih.gov/entrezo).

In general, coronaviruses, such as EMCR-CoV can be isolated and identified according to the following protocol:

### Specimen Collection

In order to find virus isolates nasopharyngeal aspirates, throat and nasal swabs, broncheo alveolar lavages, serum and plasma samples, and stools preferably from mammals such as humans, carnivores (dogs, cats, mustellits, seals etc.), horses, ruminants (cattle, sheep, goats etc.), pigs, rabbits, birds (poultry, ostriches, etc) should be examined. From birds cloaca swabs and droppings can be examined as well. Sera should be collected for immunological assays, such as ELISA, molecular-based assays, such as RT-PCR and virus neutralisation assays. Collected virus specimens may be diluted with 5 ml Dulbecco MEM medium (BioWhittaker, Walkersville, Md.) and thoroughly mixed on a vortex mixer for one minute. The suspension is thus centrifuged for ten minutes at 840×g. The sediment is spread on a multispot slide (Nutacon, Leimuiden, The Netherlands) for immunofluorescence techniques, and the supernatant is used for virus isolation.

### Virus Isolation

For virus isolation Vero-118 cells or tMK cells (RIVM, Bilthoven, The Netherlands) were cultured in 24 well plates containing glass slides (Costar, Cambridge, UK), with the medium described below supplemented with 10% fetal bovine serum (BioWhittaker, Vervier, Belgium). Before inoculation the plates were washed with PBS and supplied with Eagle's MEM with Hanks' salt (ICN, Costa mesa, CA) supplemented with 0.52/liter gram NaHCO₃, 0.025 M Hepes (Biowhittaker), 2 mM L-glutamine (Biowhittaker), 200 units/liter penicilline, 200 μg/liter streptomycine (Biowhittaker), 1 gram/liter lactalbumine (Sigma-Aldrich, Zwijndrecht, The Netherlands), 2.0 gram/liter D-glucose (Merck, Amsterdam, The Netherlands), 10 gram/liter peptone (Oxoid, Haarlem, The Netherlands) and 0.02% trypsine (Life Technologies, Bethesda, Md.). The plates were inoculated with supernatant of the patient samples, 0,2 ml per well in triplicate, followed by centrifuging at 840×g for one hour. After inoculation the plates were incubated at 37° C. for 1-7 days and cultures were checked daily for CPE. Extensive CPE was generally observed within 5-10 and included detachment of cells from the monolayer.

### Virus Culture

Sub-confluent monolayers of tMK cells or Vero clone 118 cells in media as described above were inoculated with supernatants of samples that displayed CPE or with samples taken from a patient.

### RNA Isolation

RNA was isolated from the supernatant of infected cell cultures or sucrose gradient fractions using a High Pure RNA Isolation kit according to instructions from the manufacturer (Roche Diagnostics, Almere, The Netherlands). RNA can also be isolated following other procedures known in the field (Current Protocols in Molecular Biology).

### Sequence Analysis

Sequence analyses were performed by BaseClear holding BV (Leiden, The Netherlands).

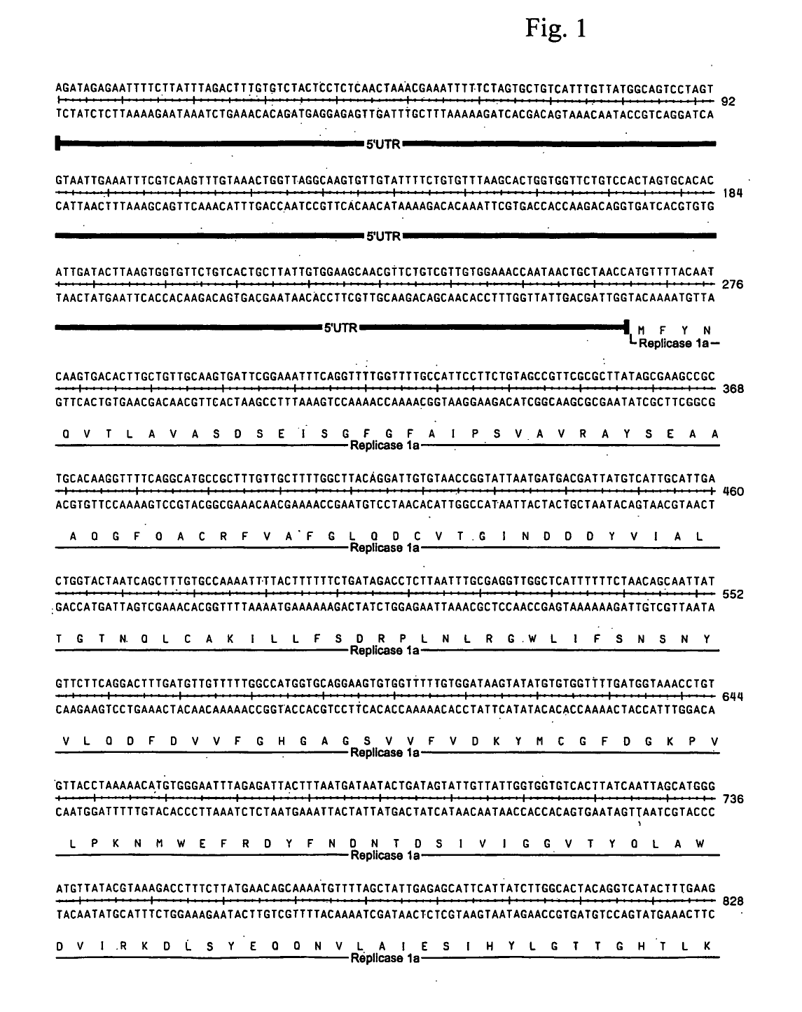

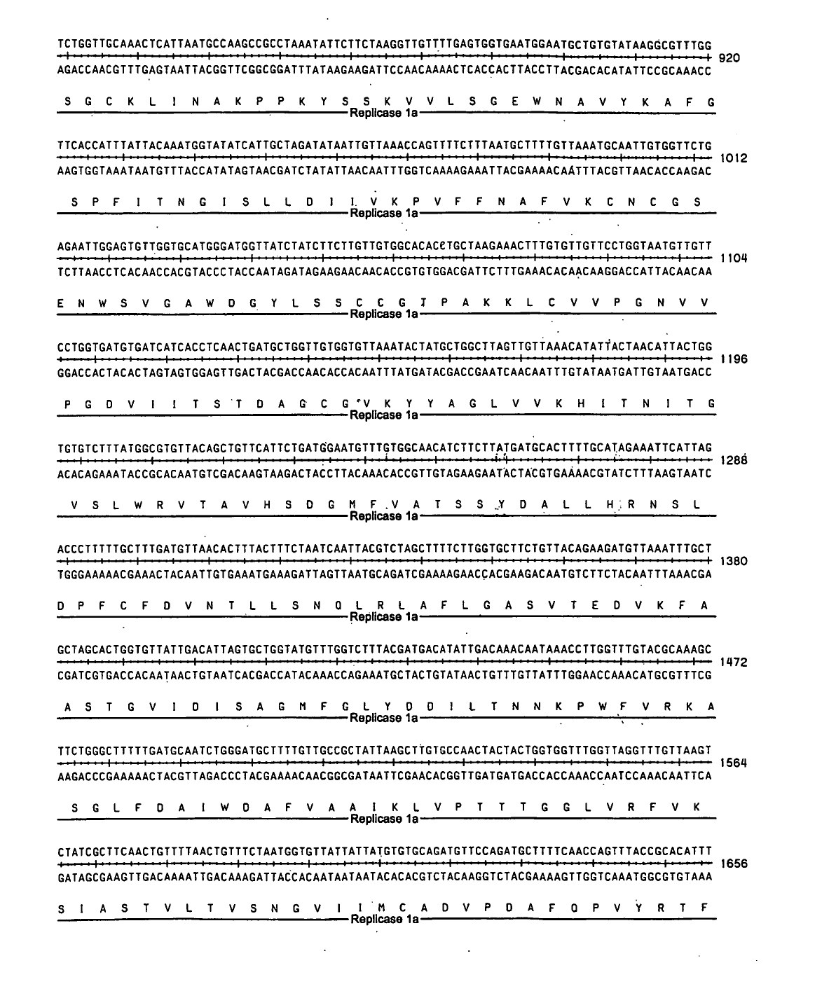

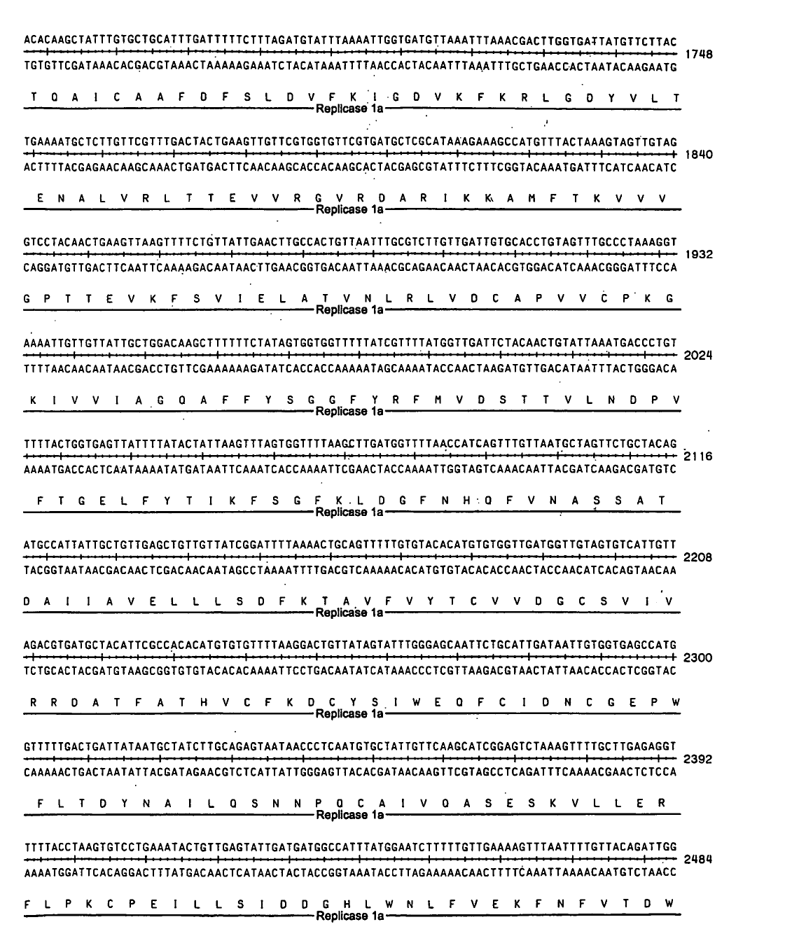

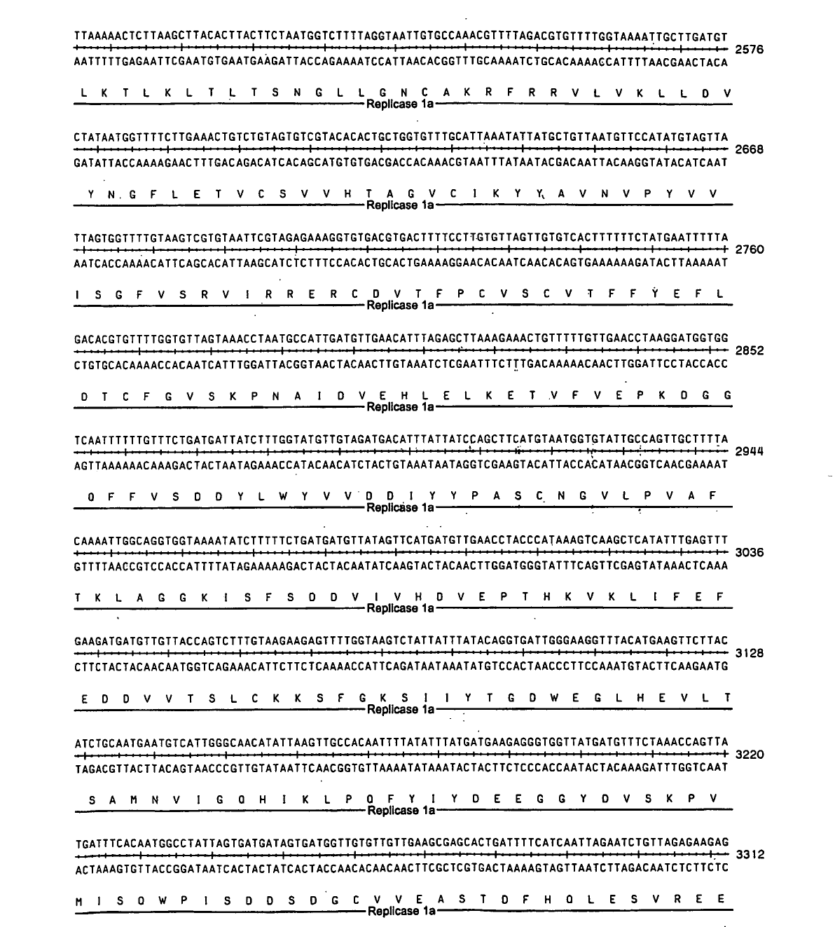

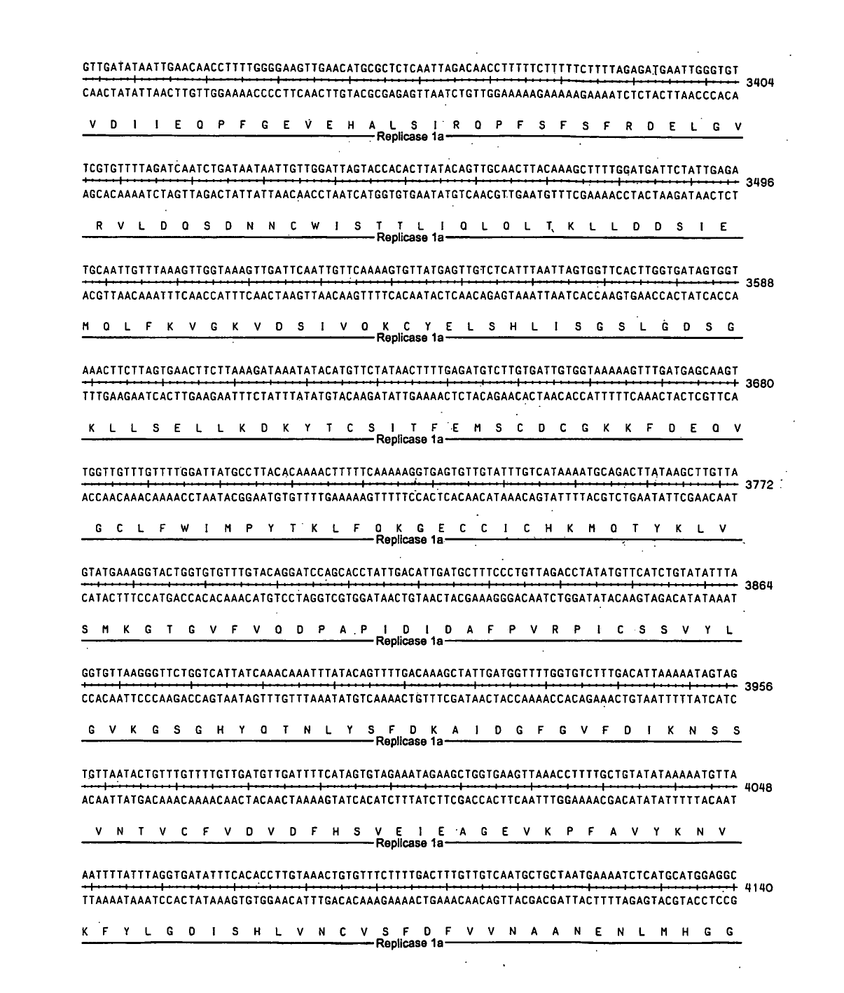

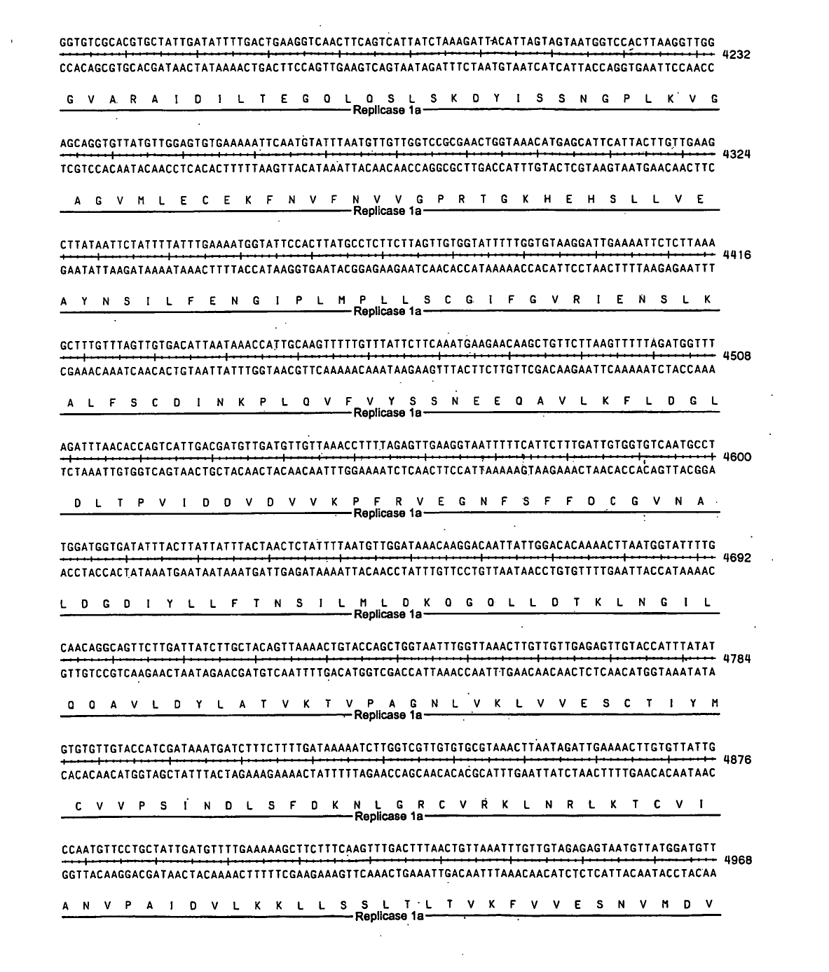

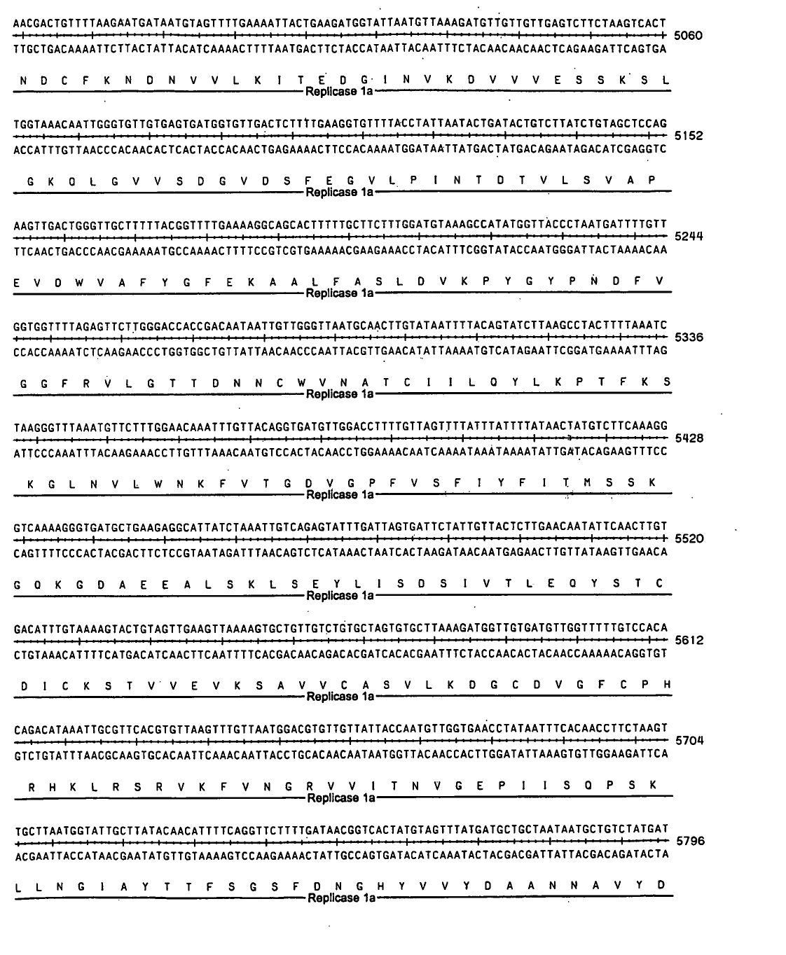

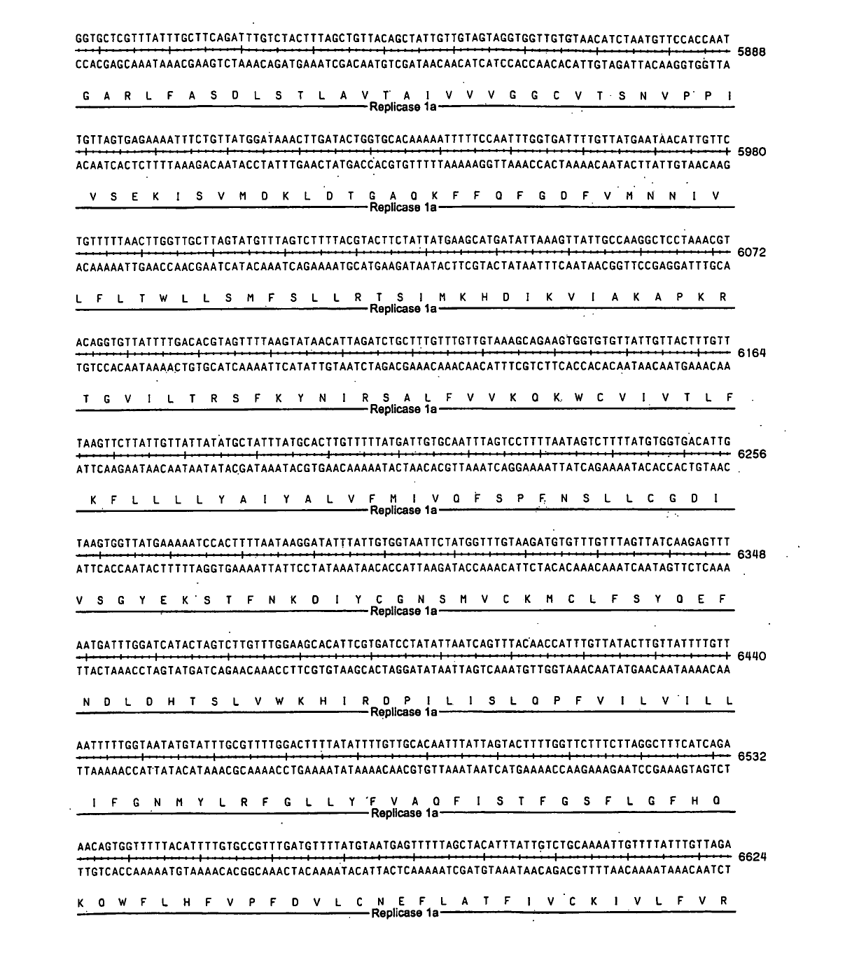

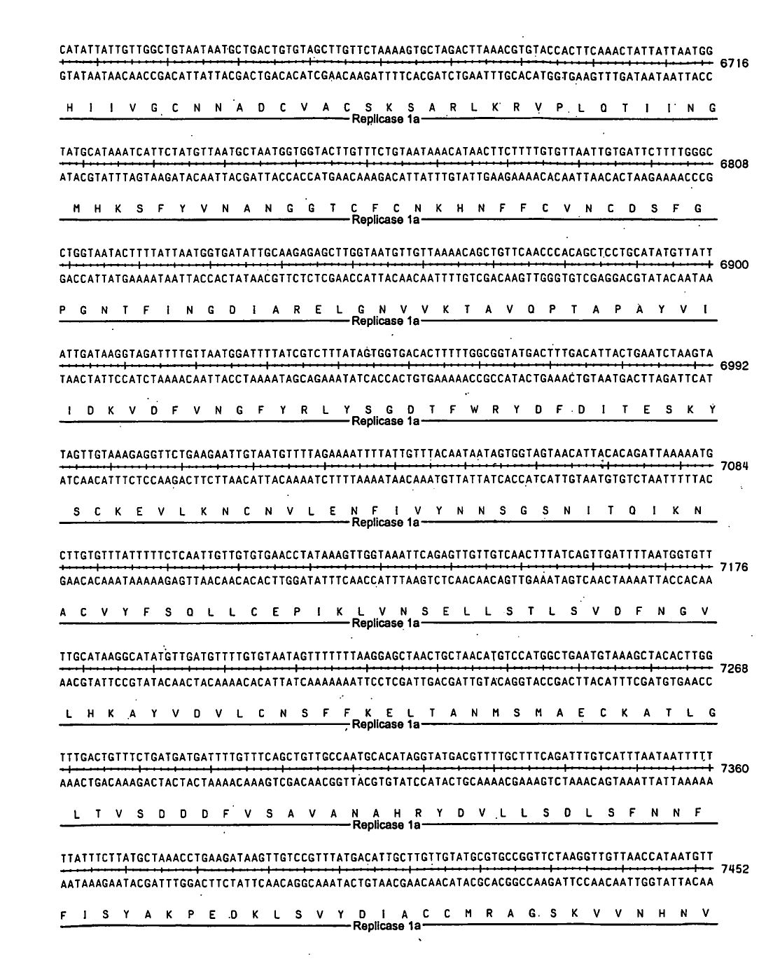

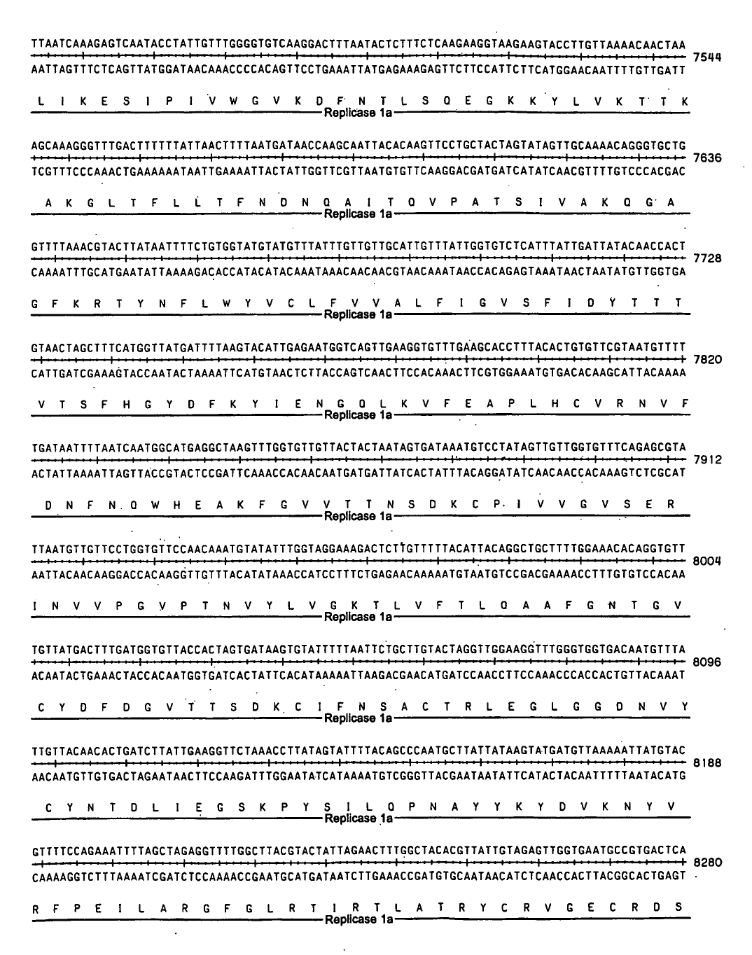

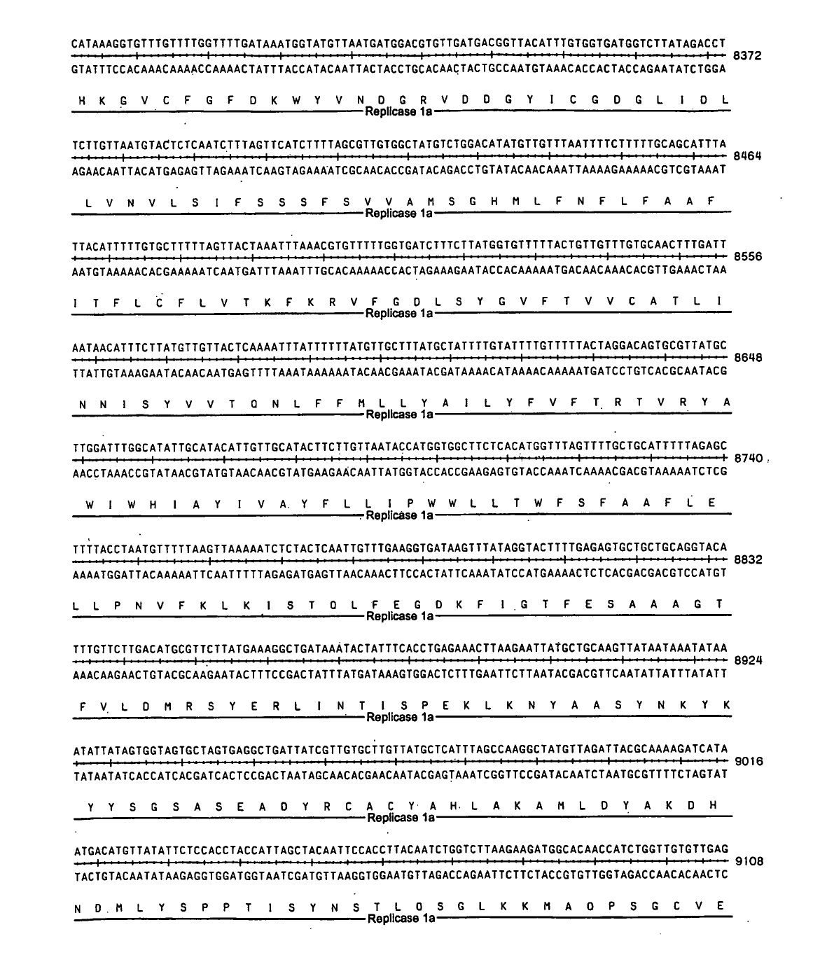

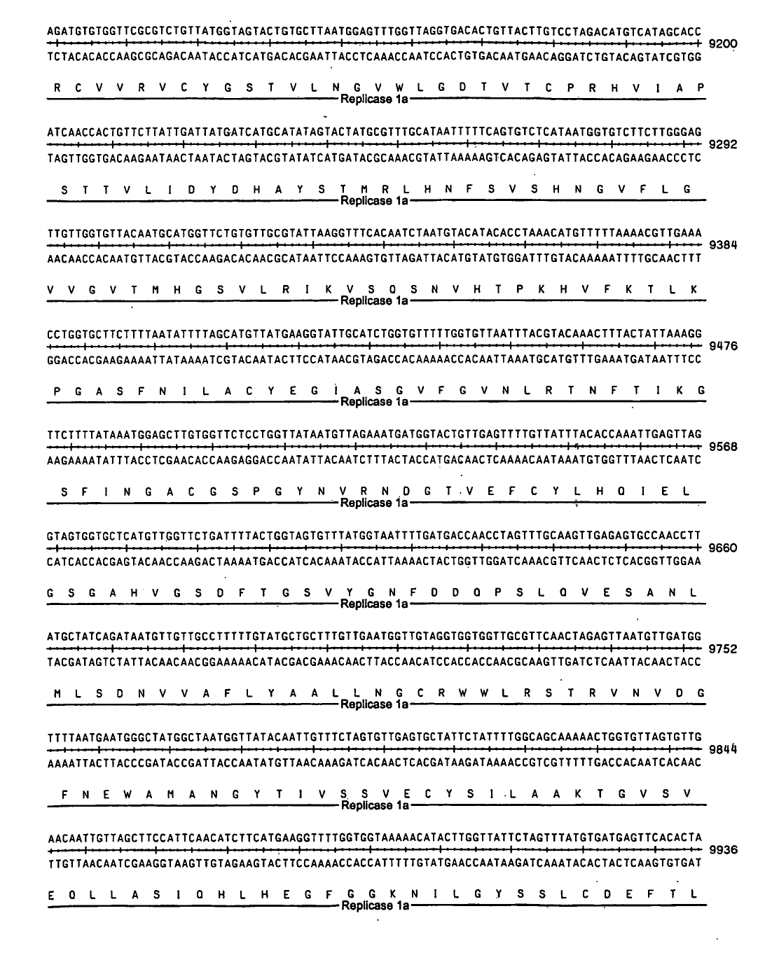

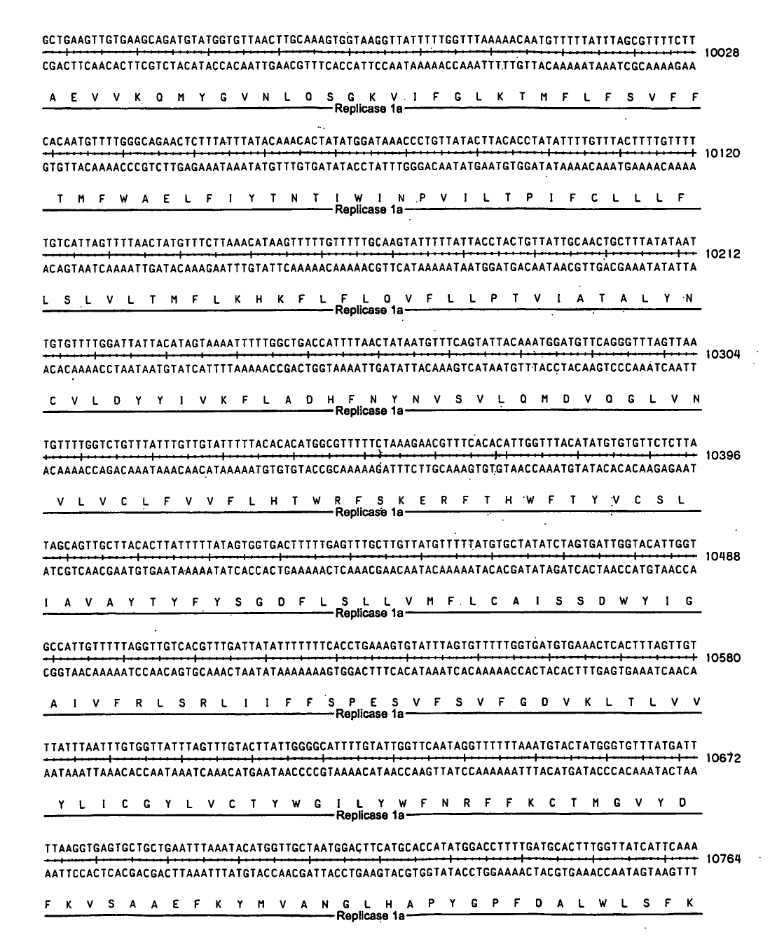

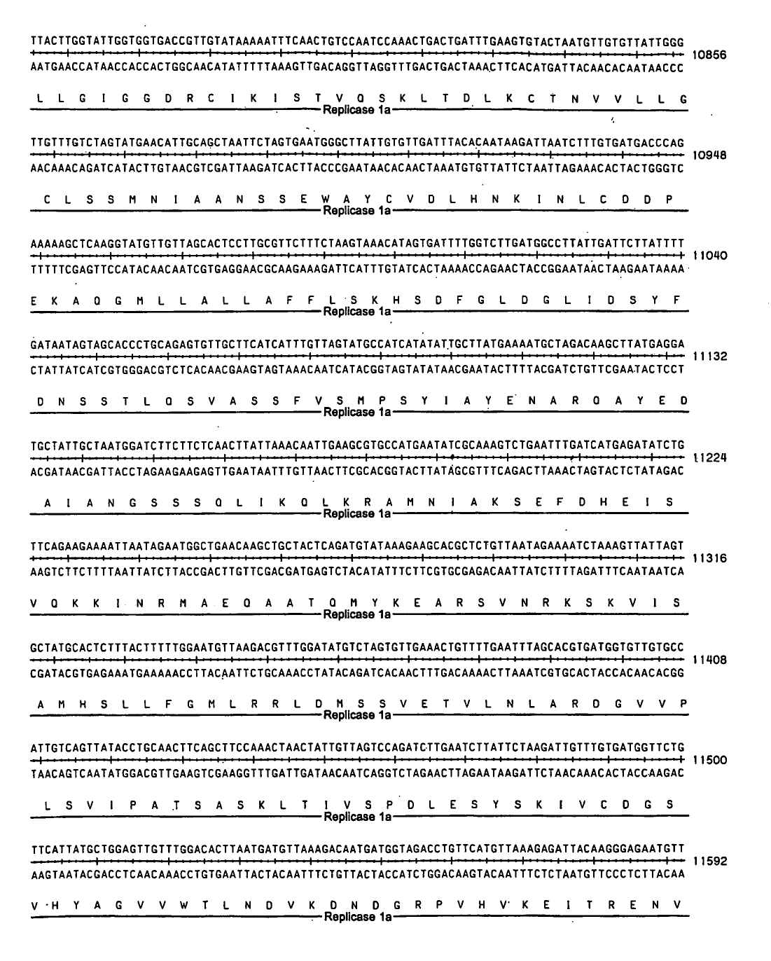

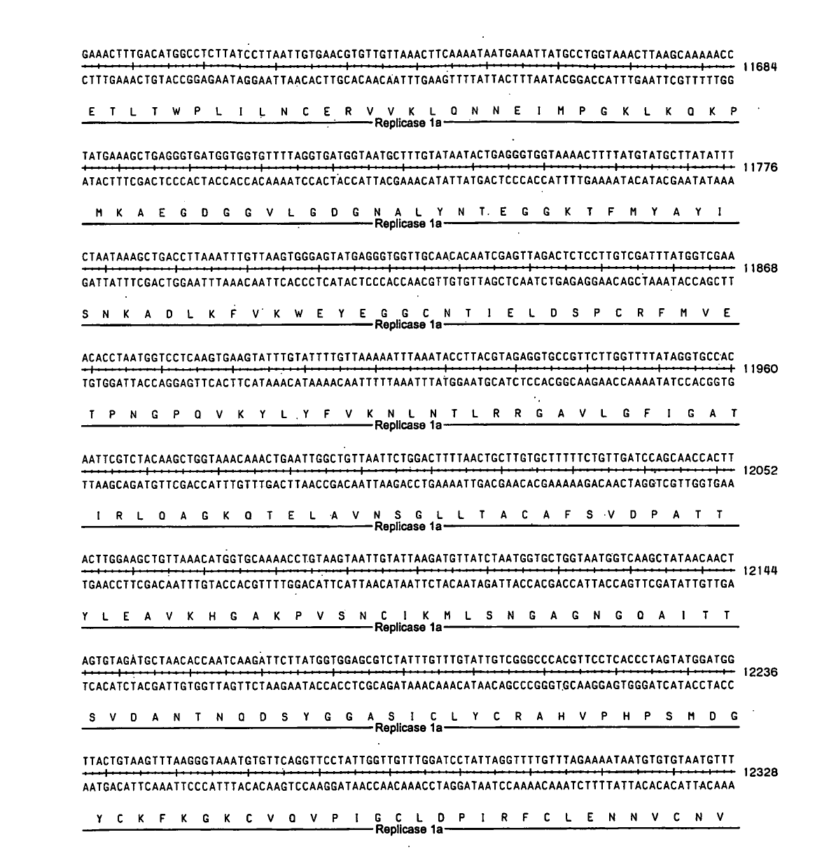

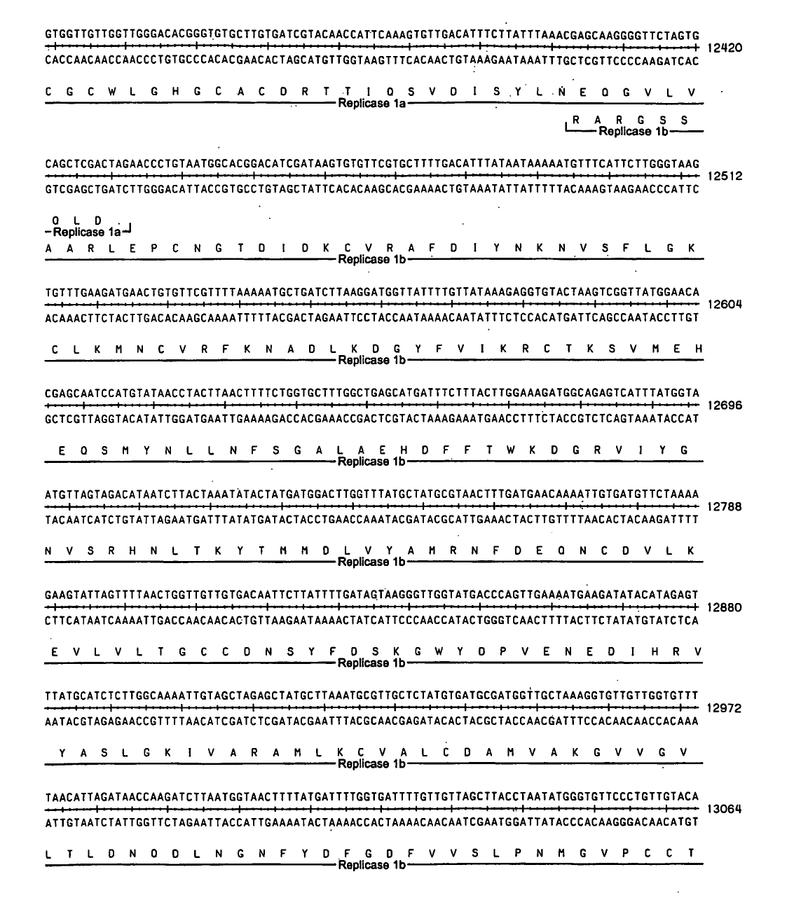

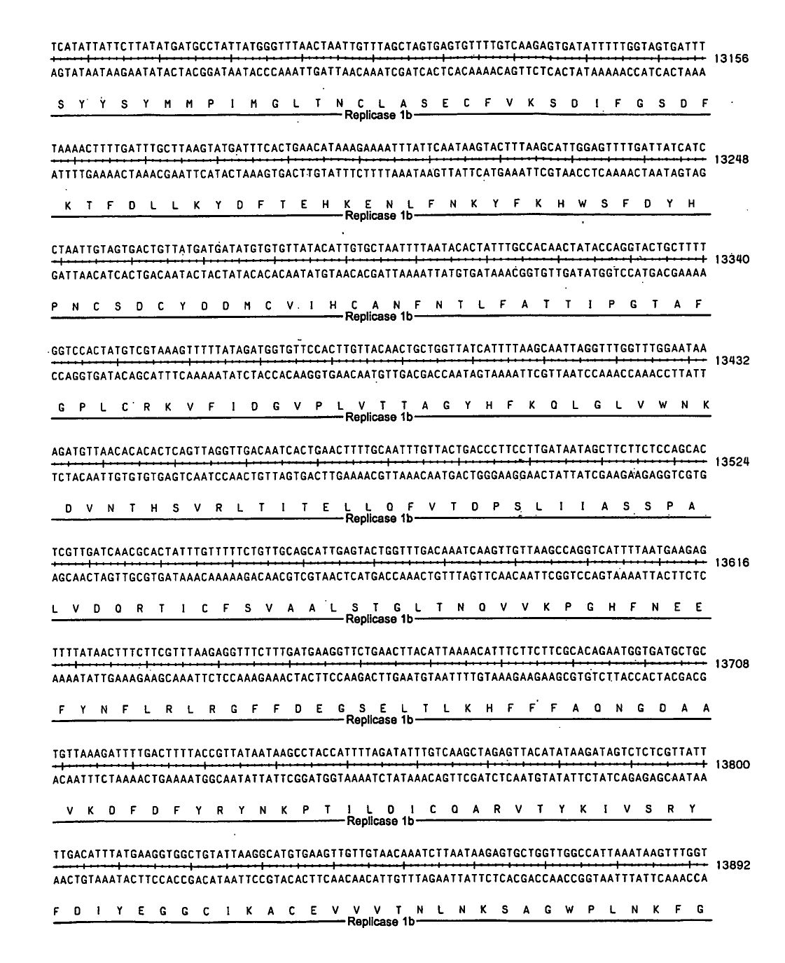

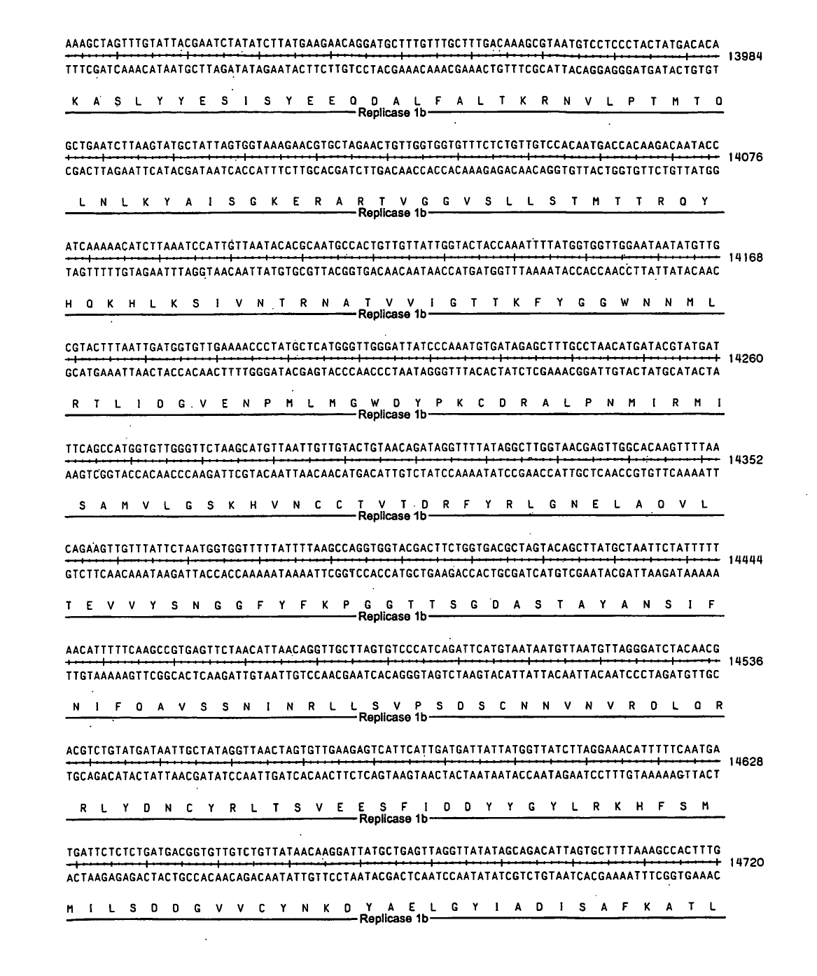

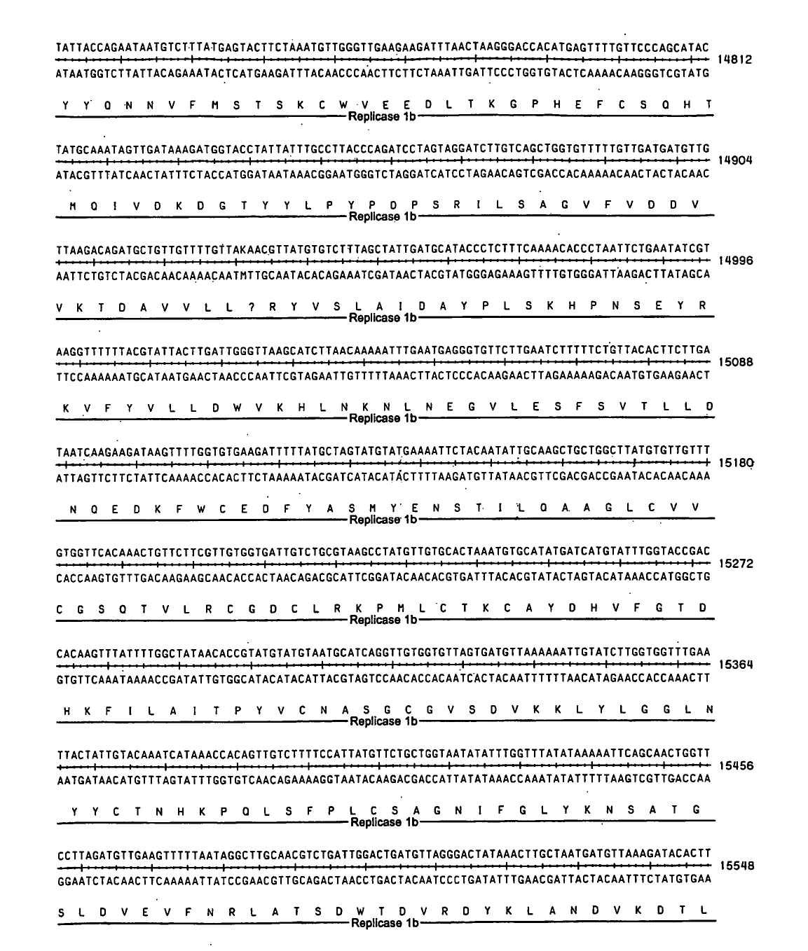

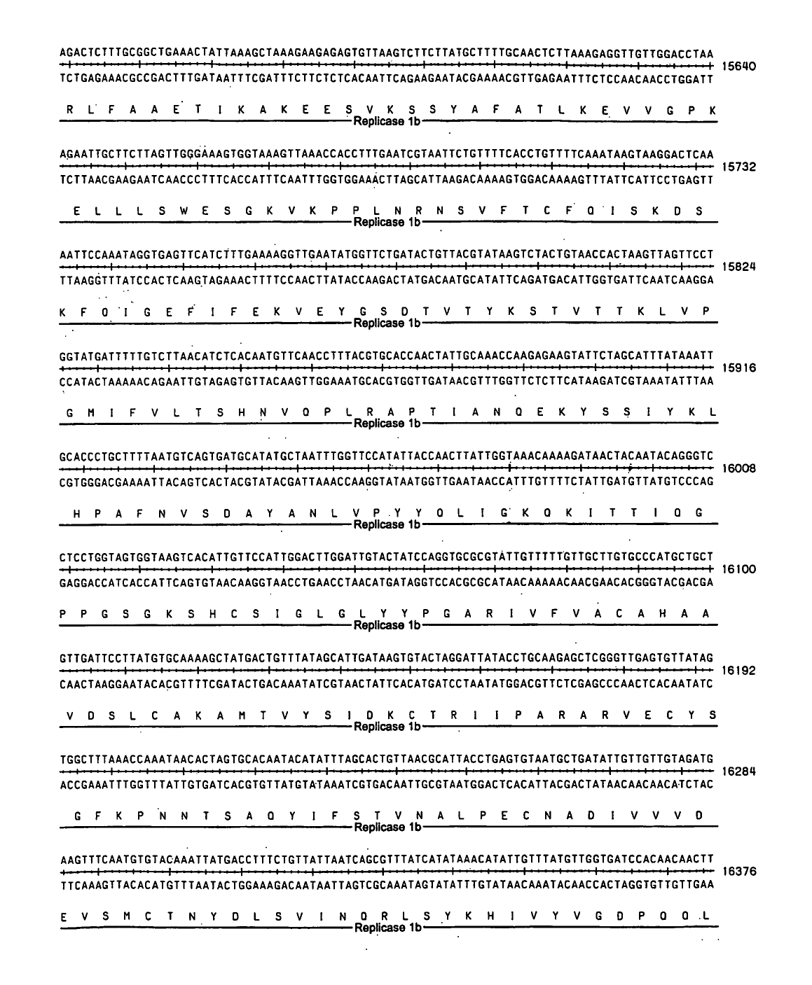

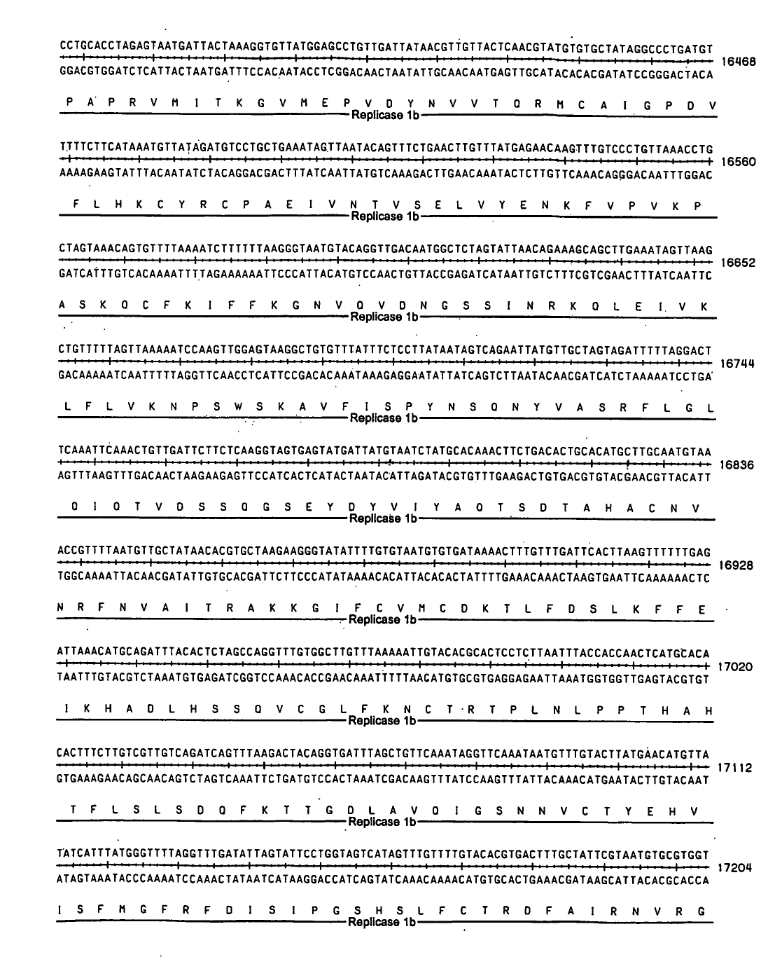

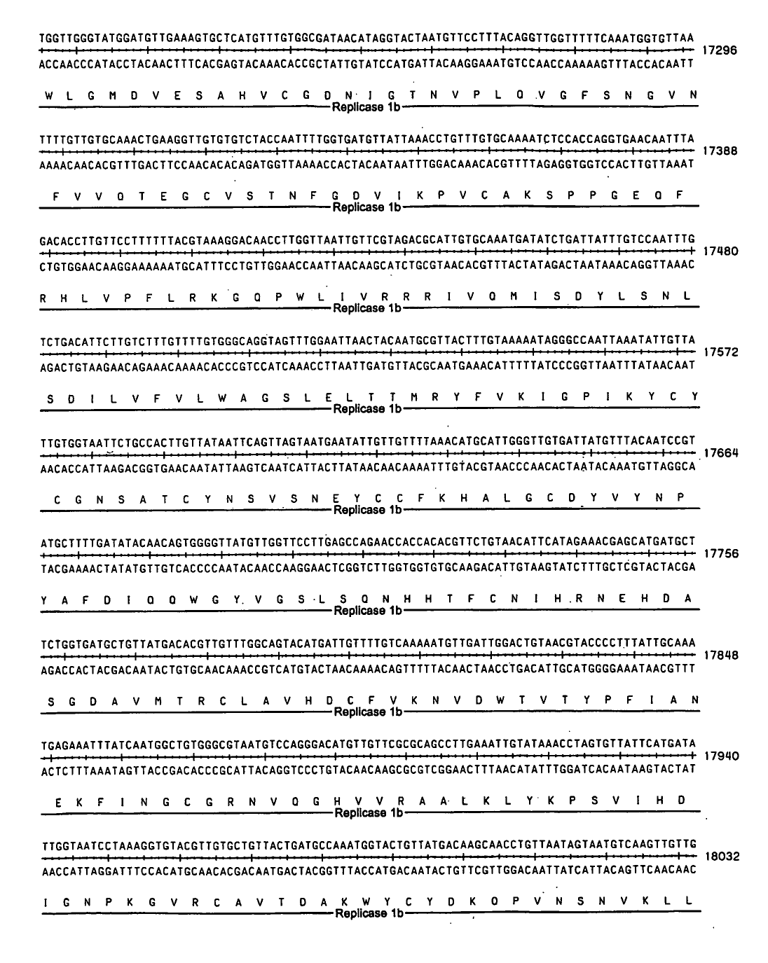

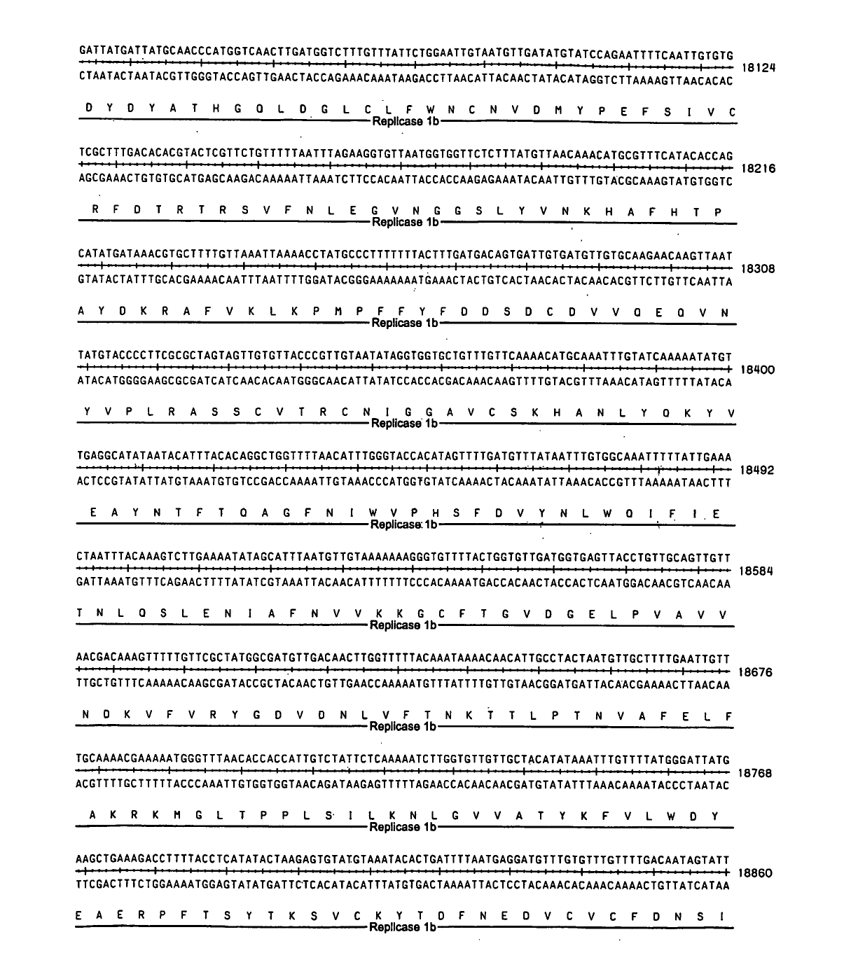

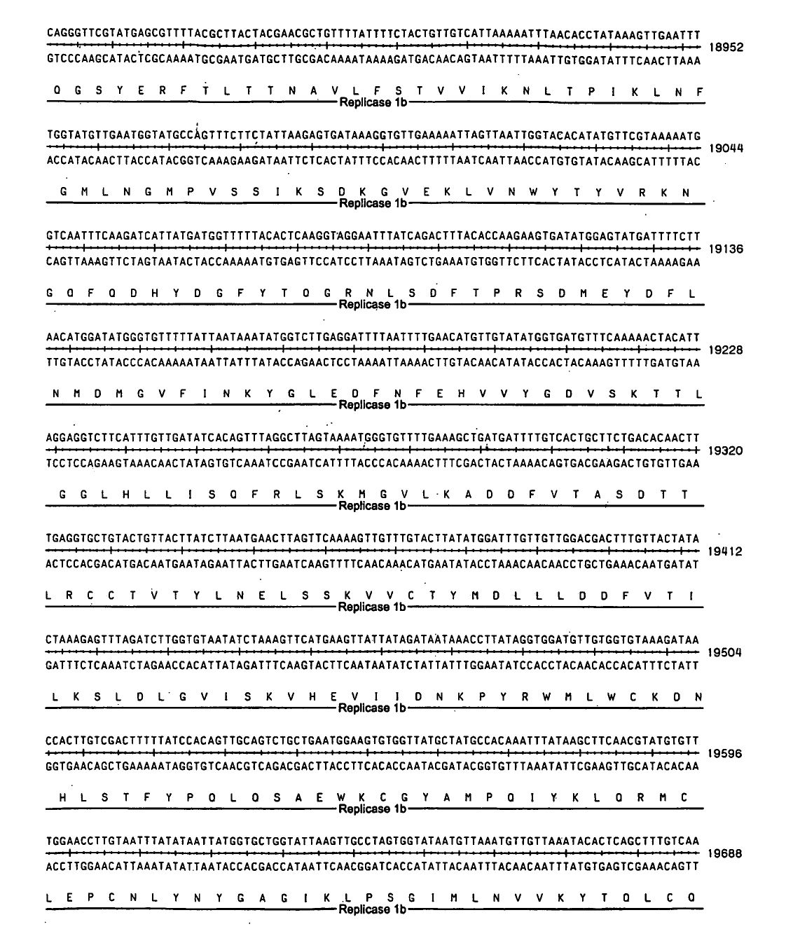

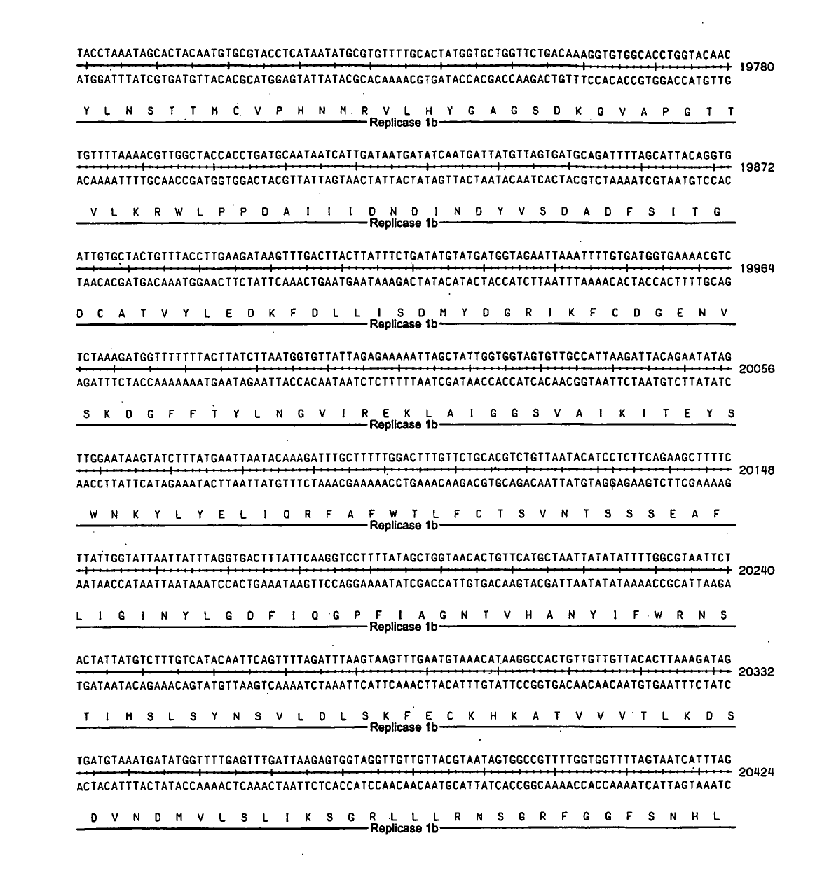

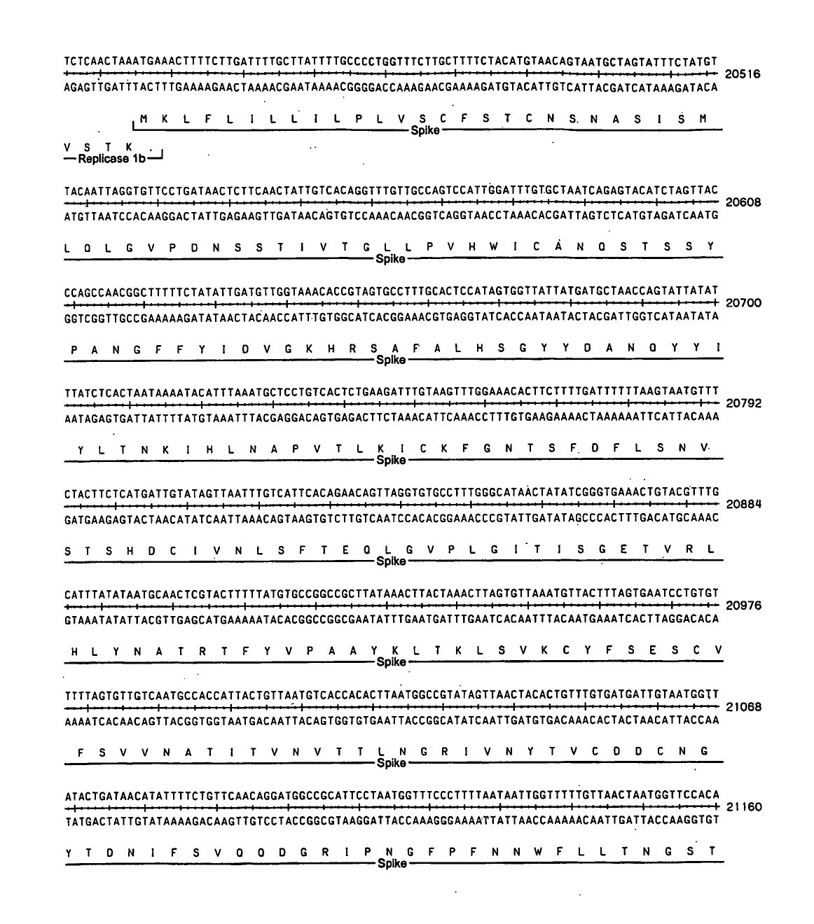

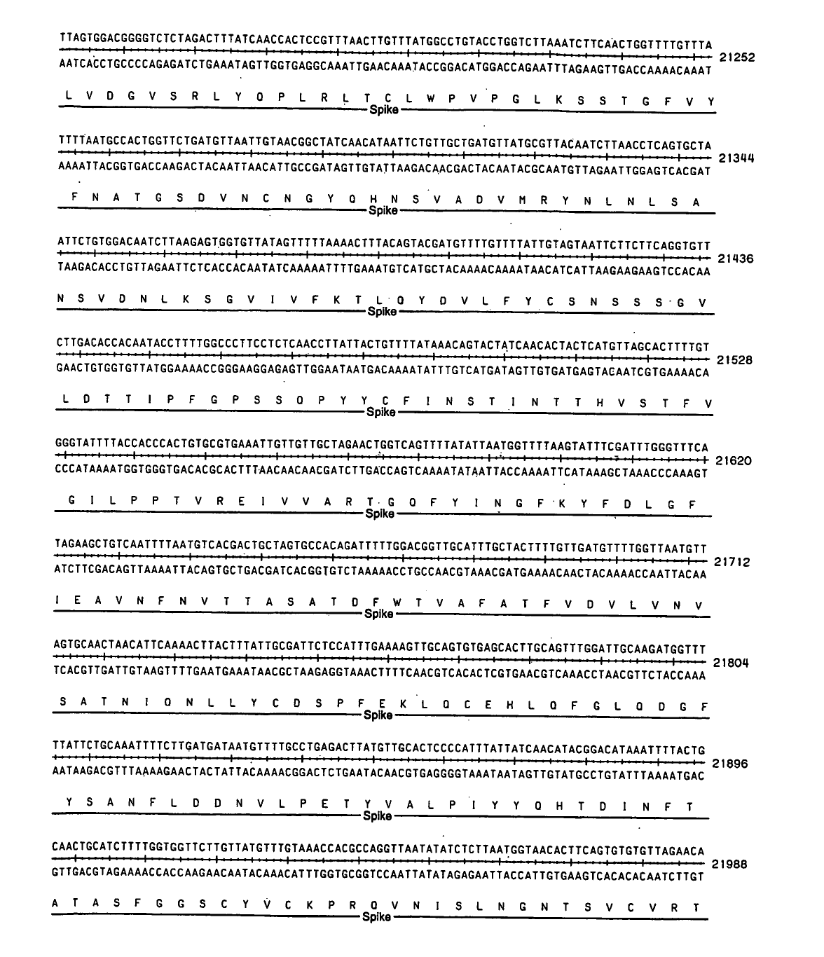

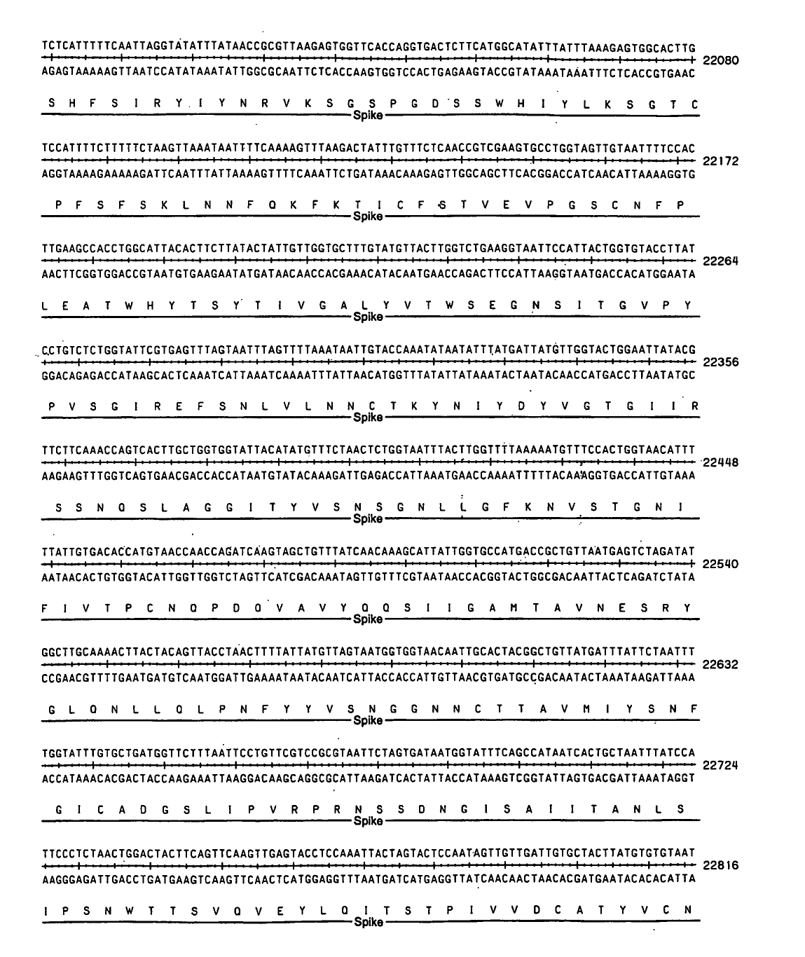

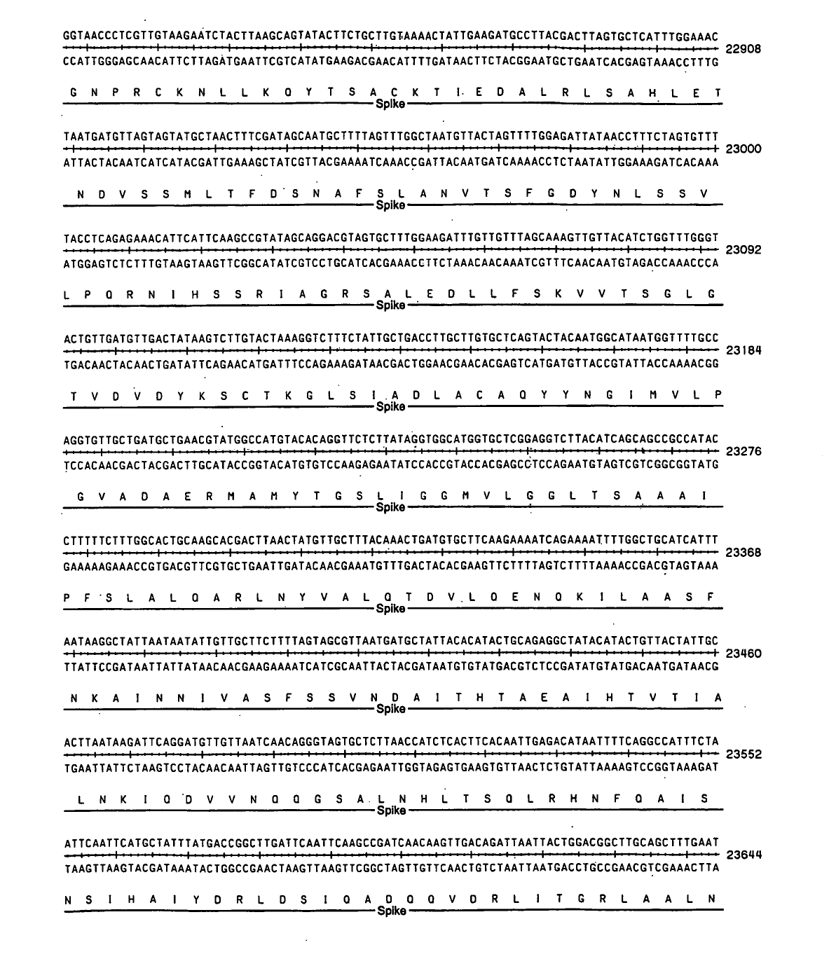

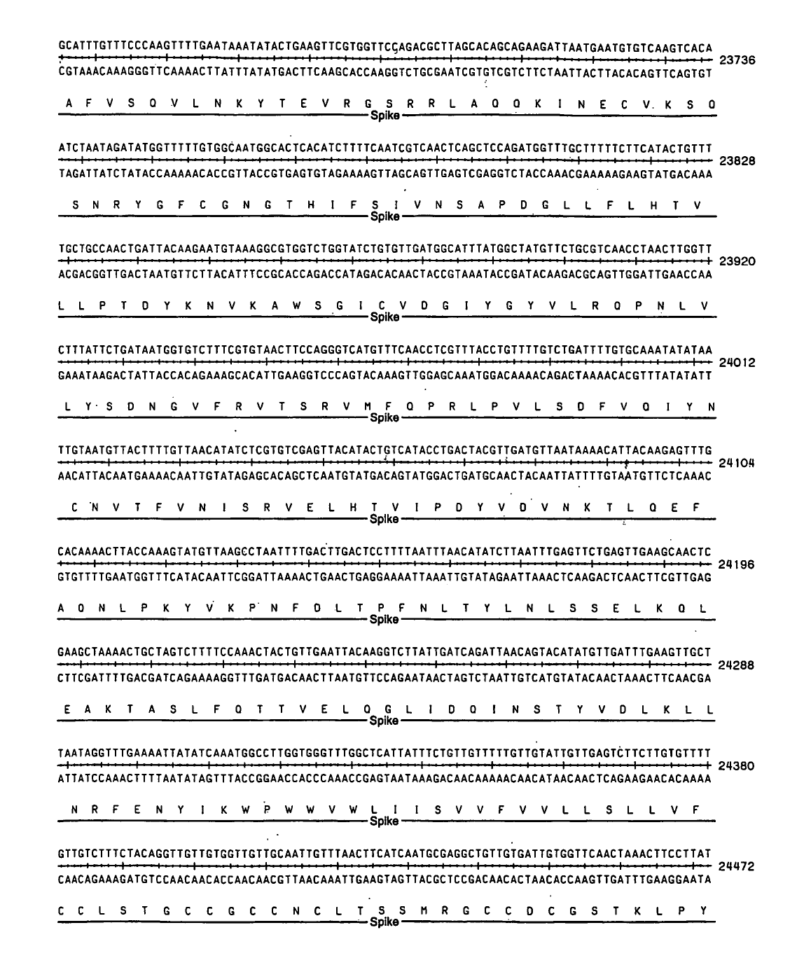

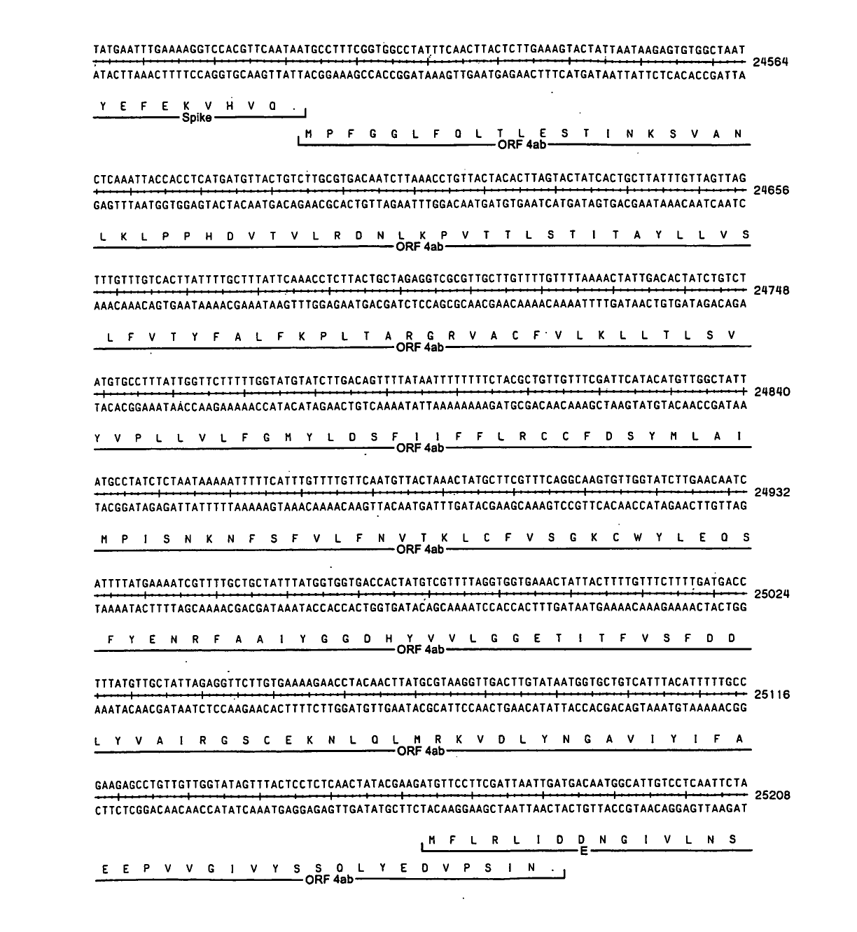

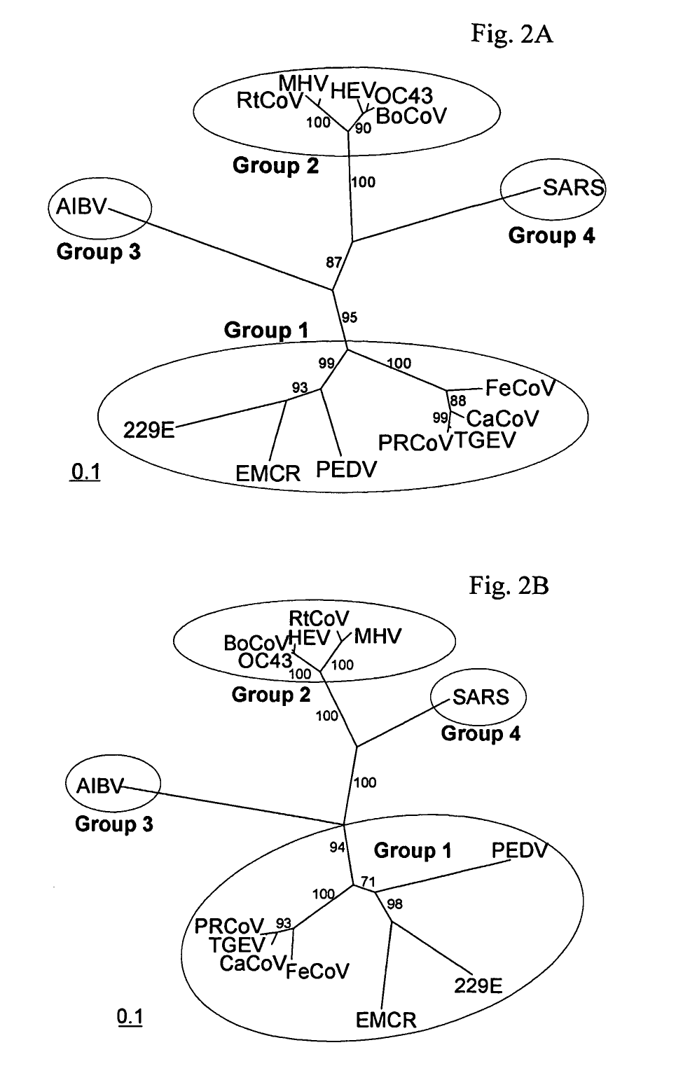

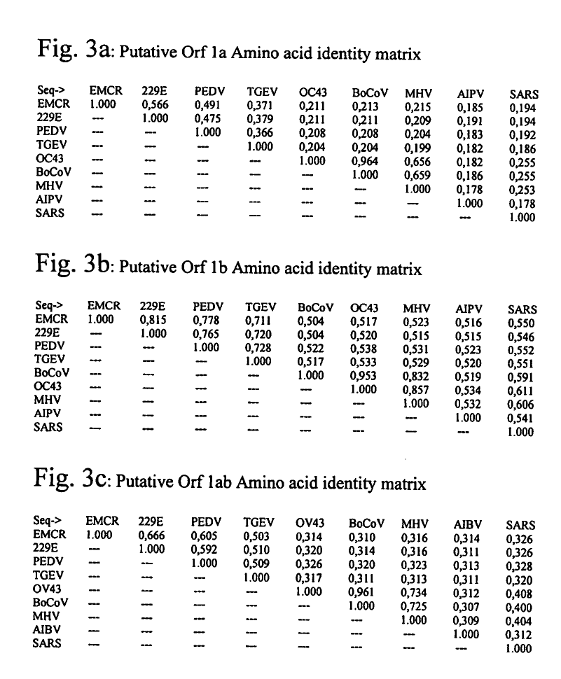

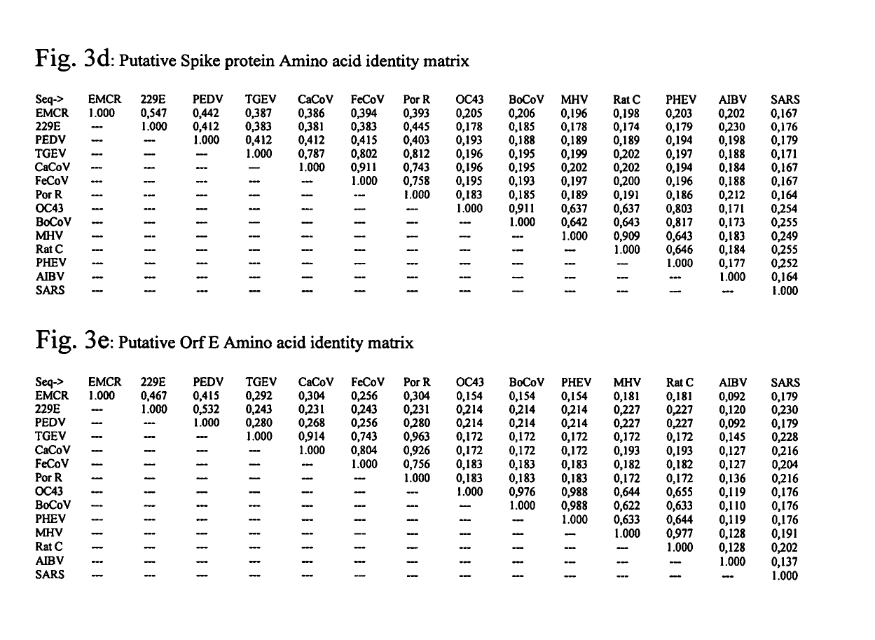

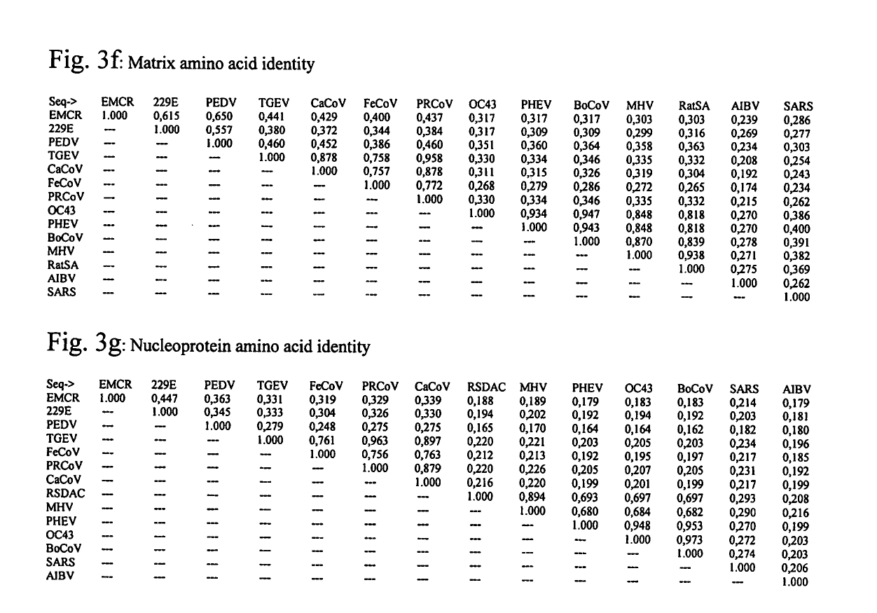

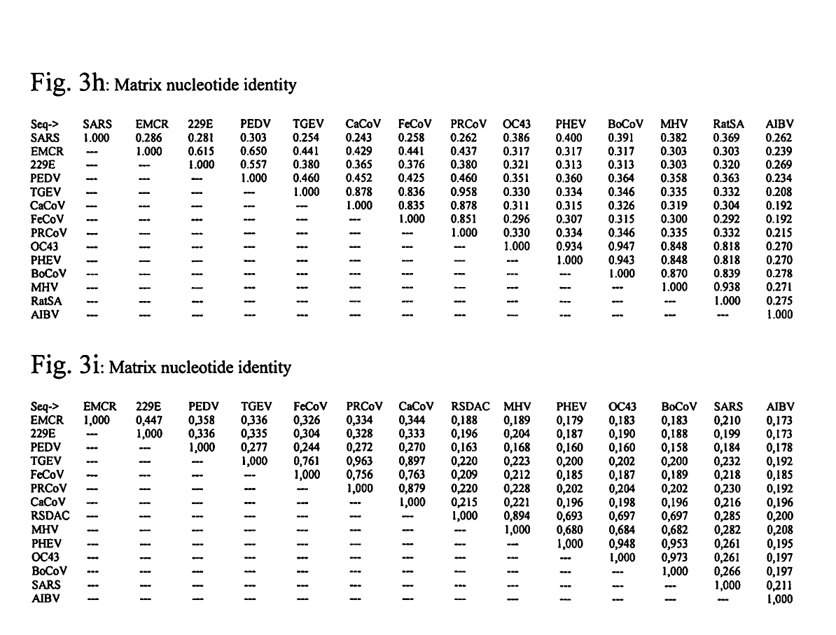

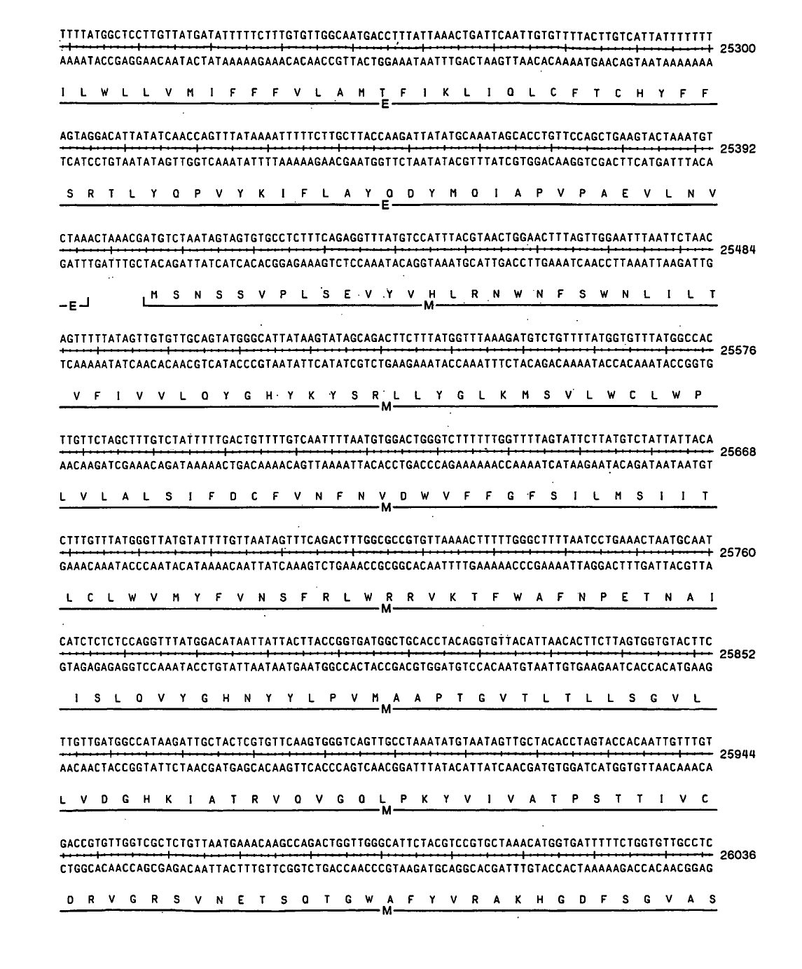

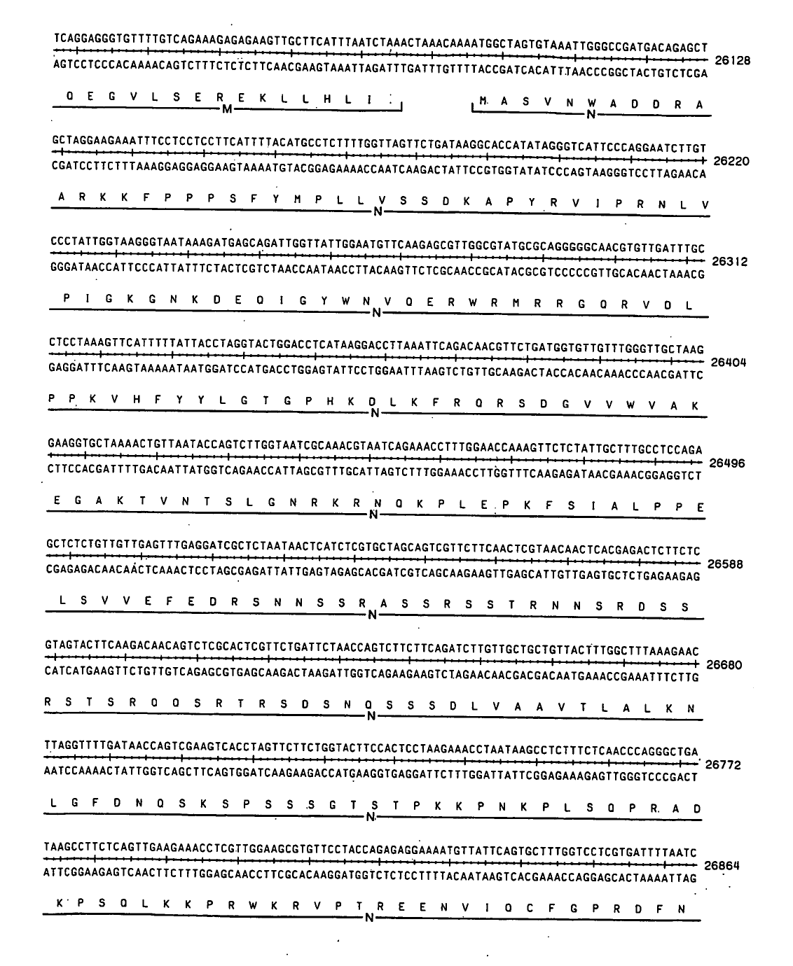

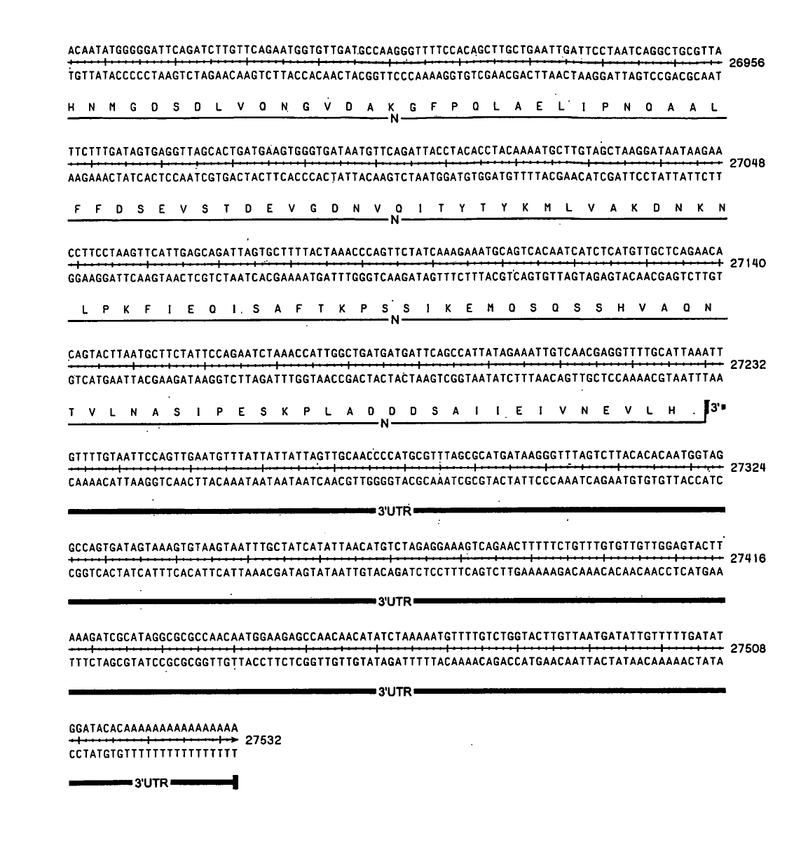

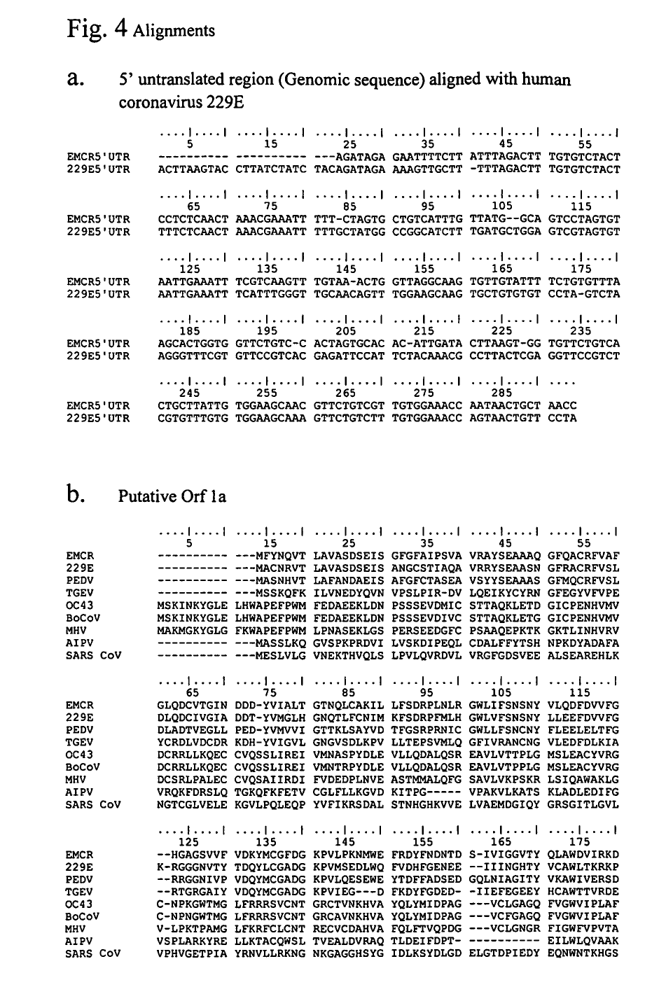

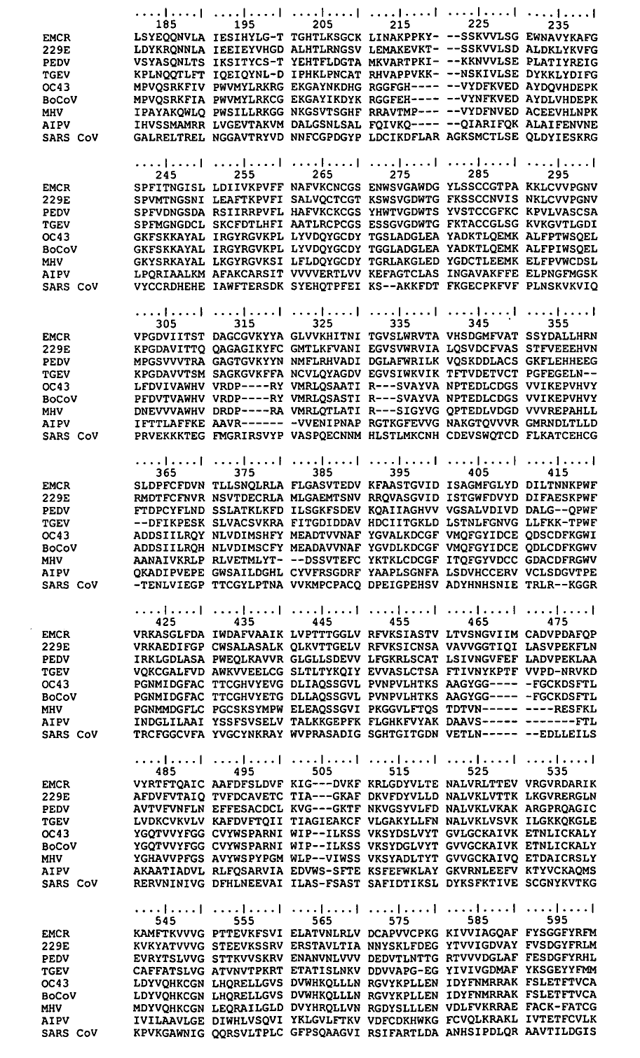

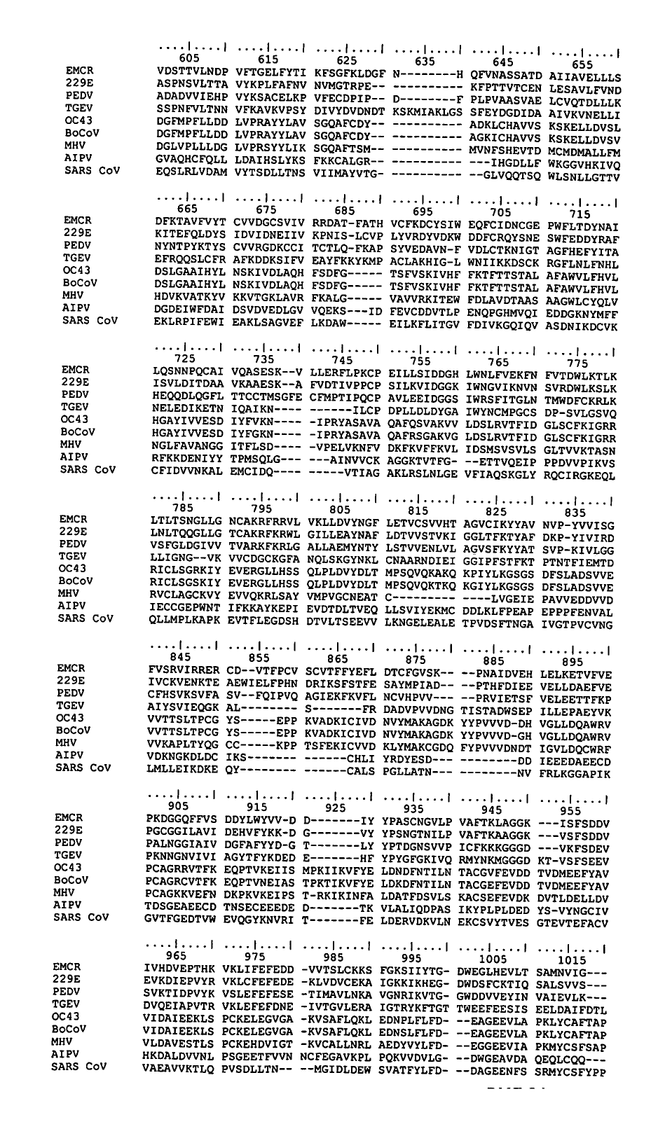

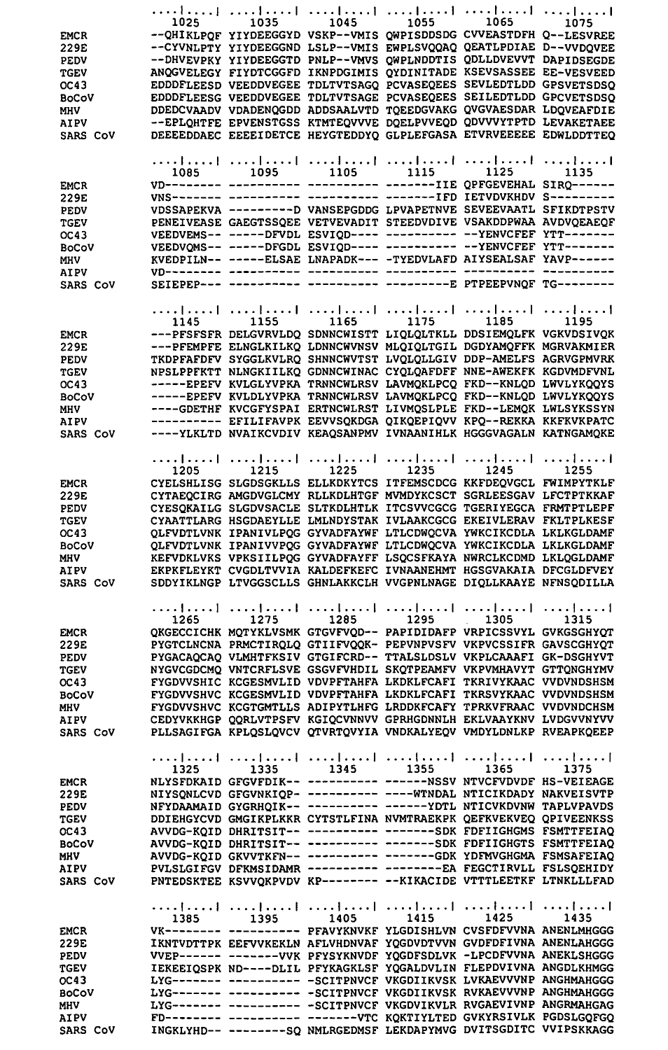

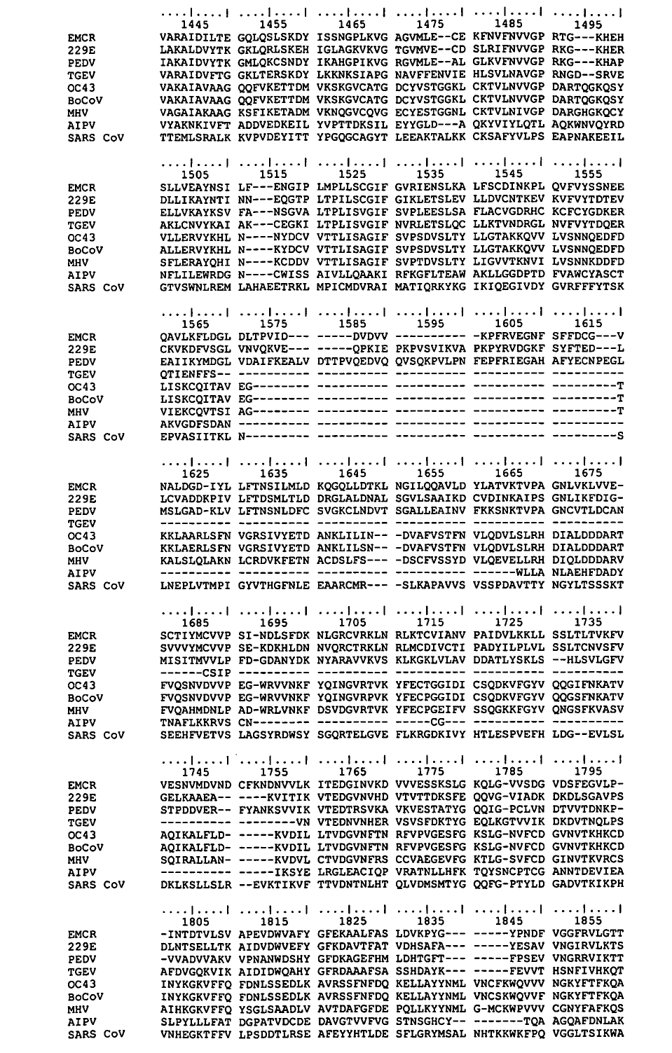

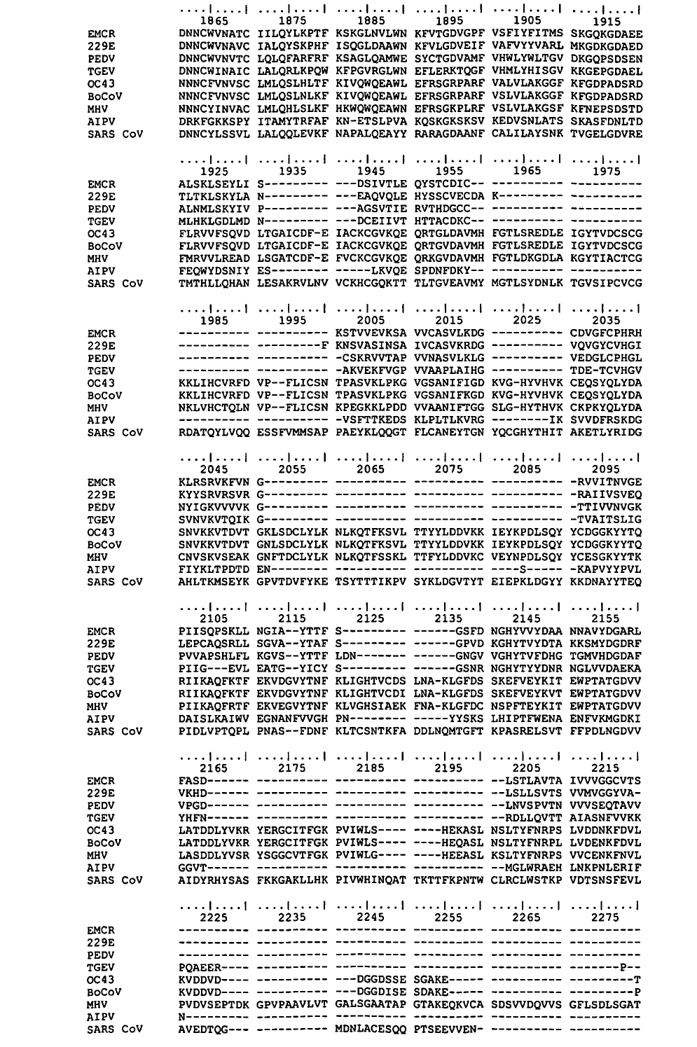

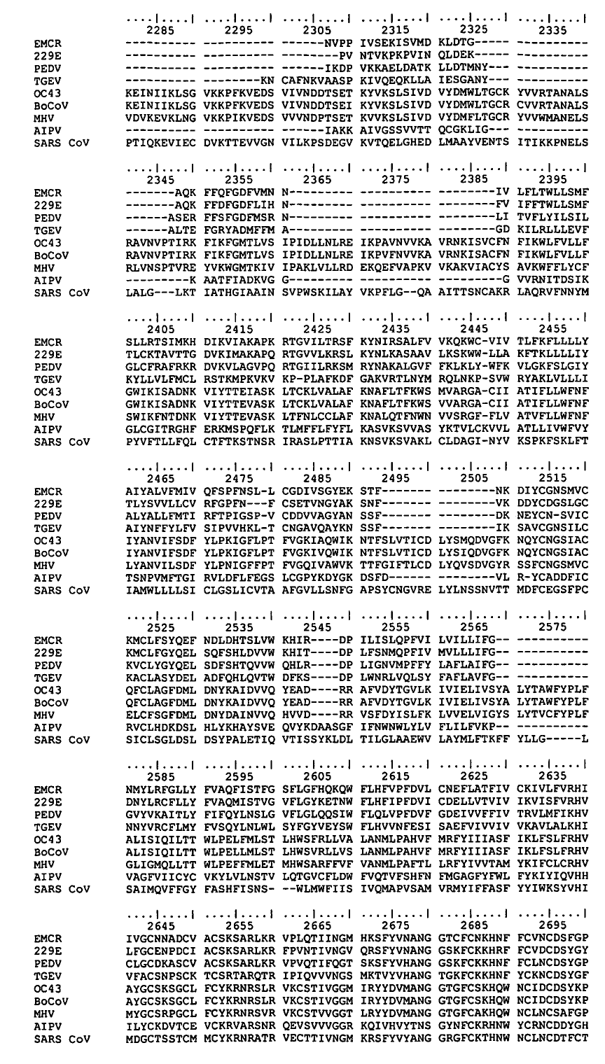

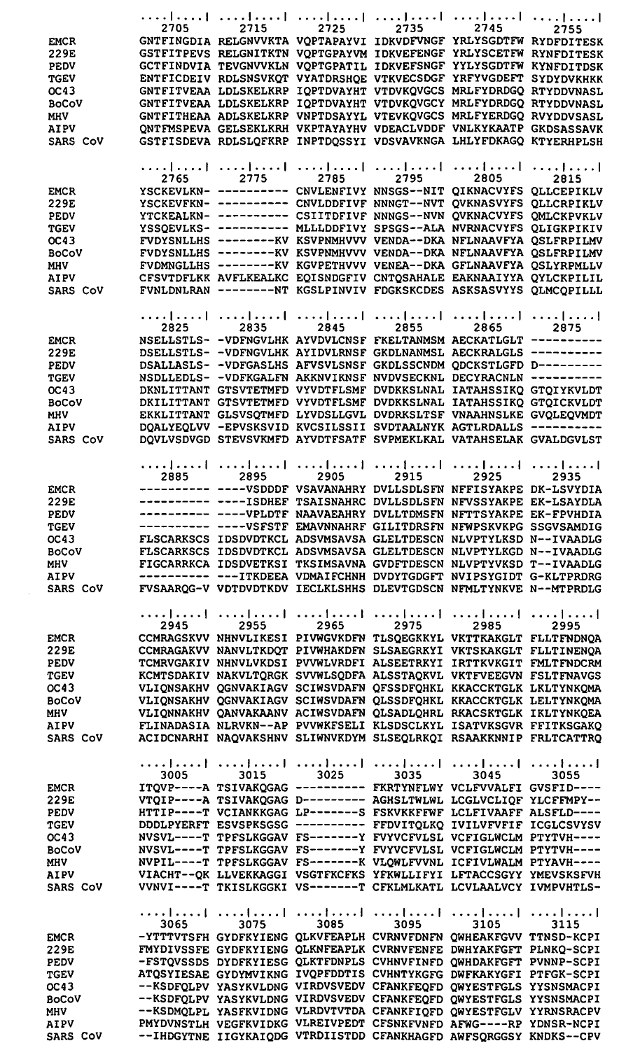

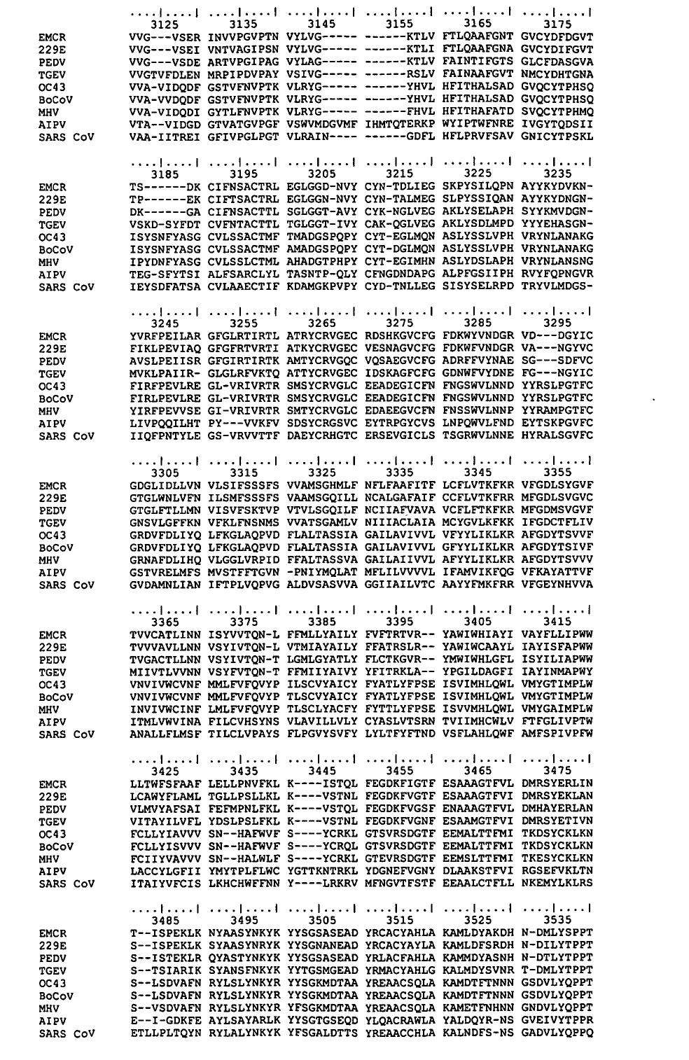

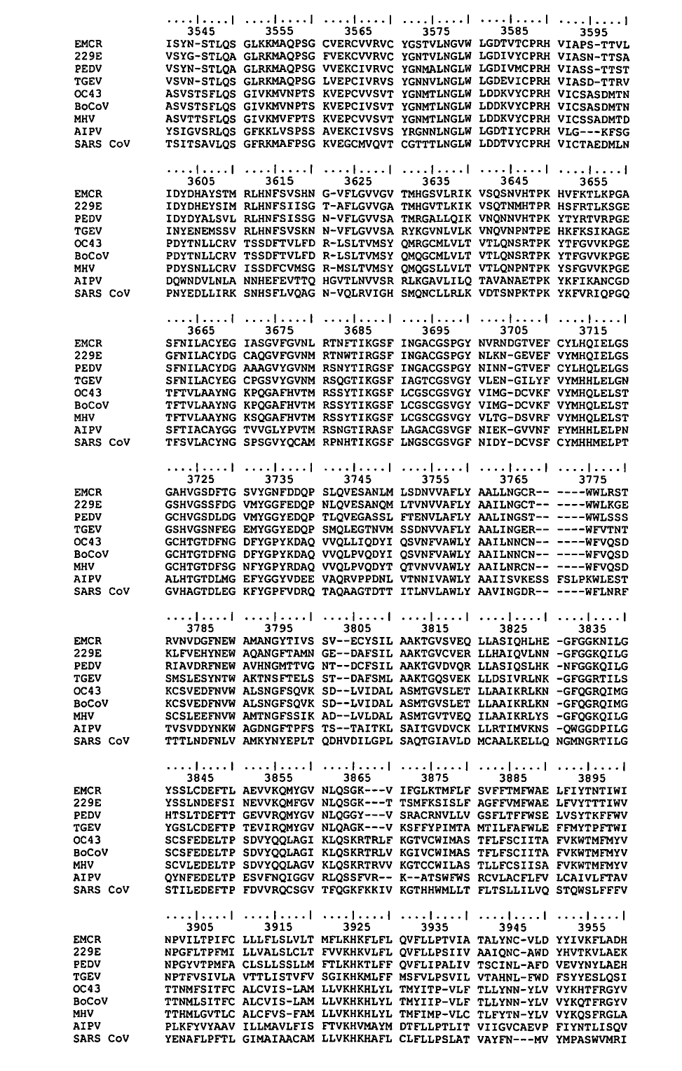

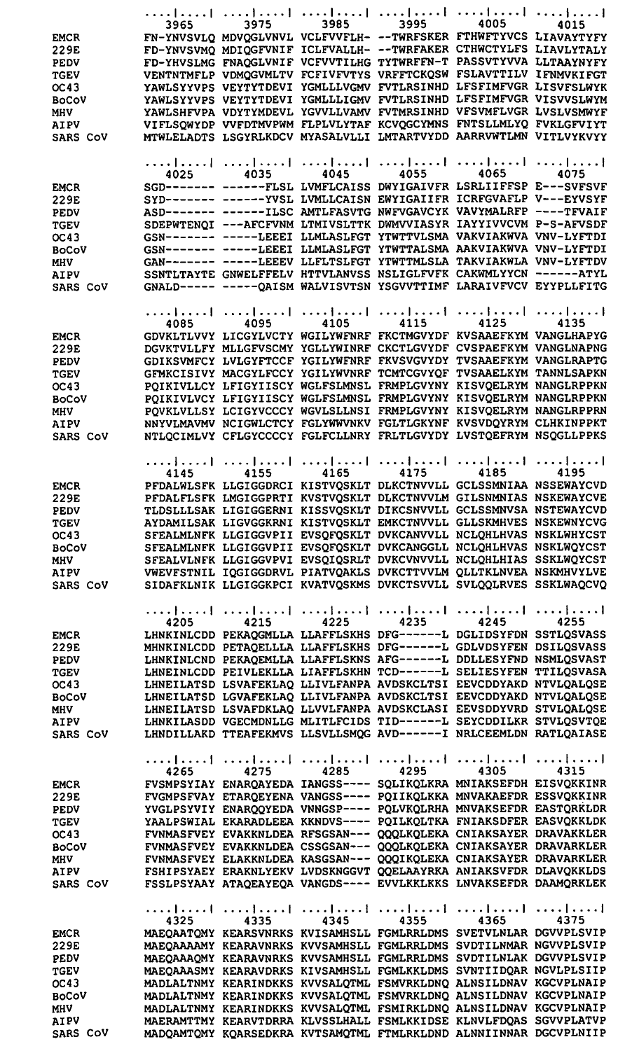

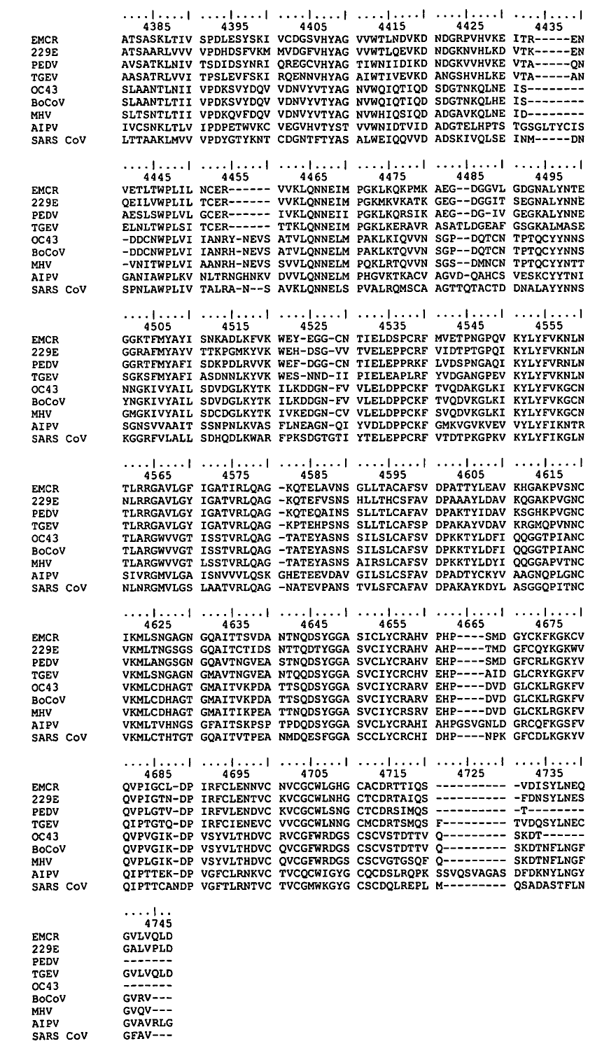

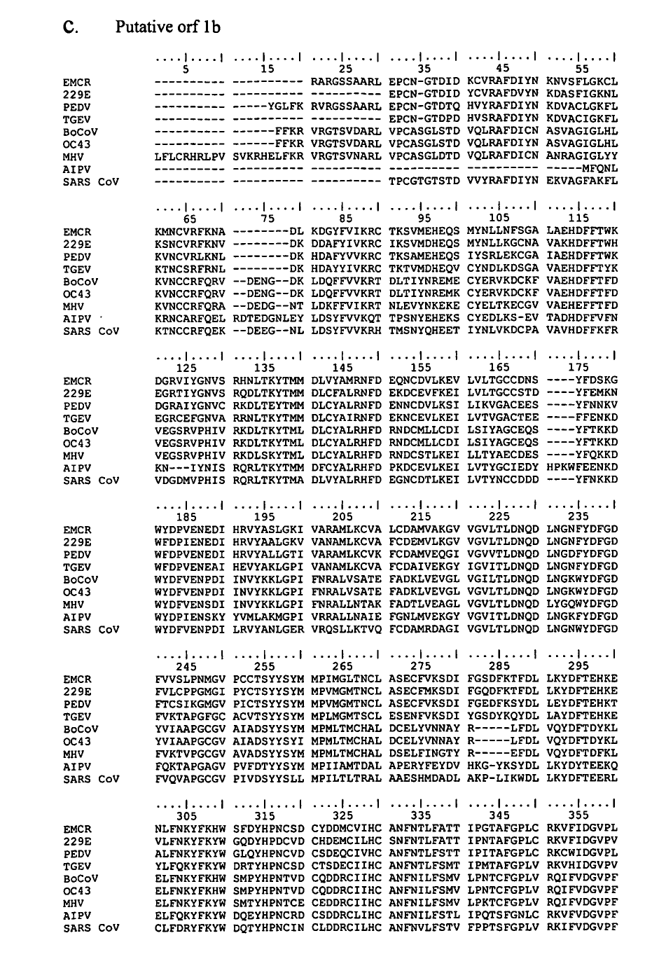

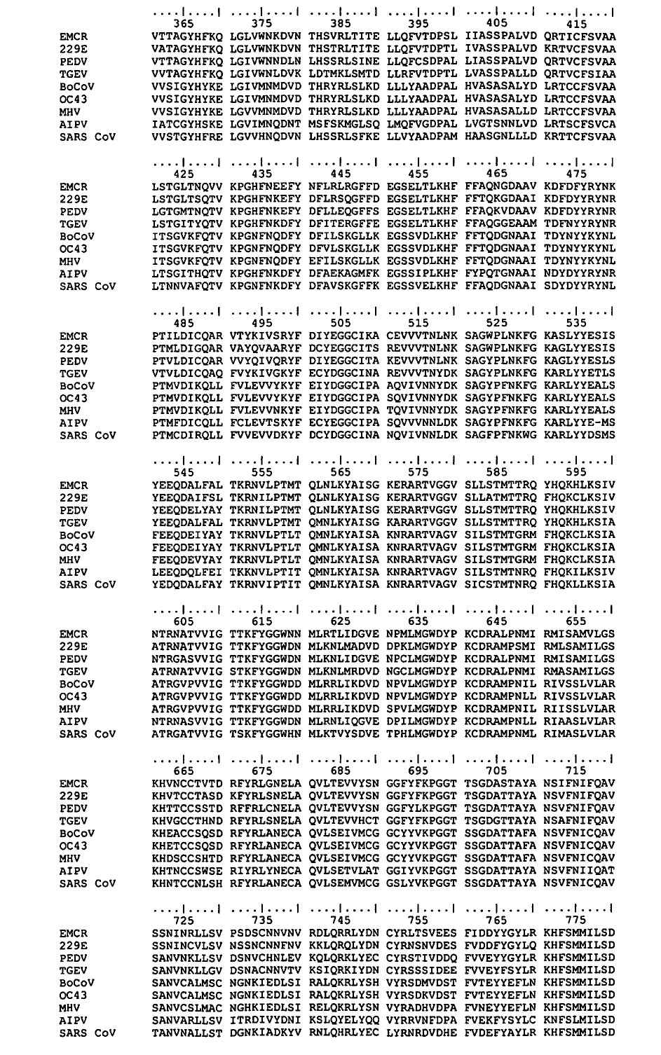

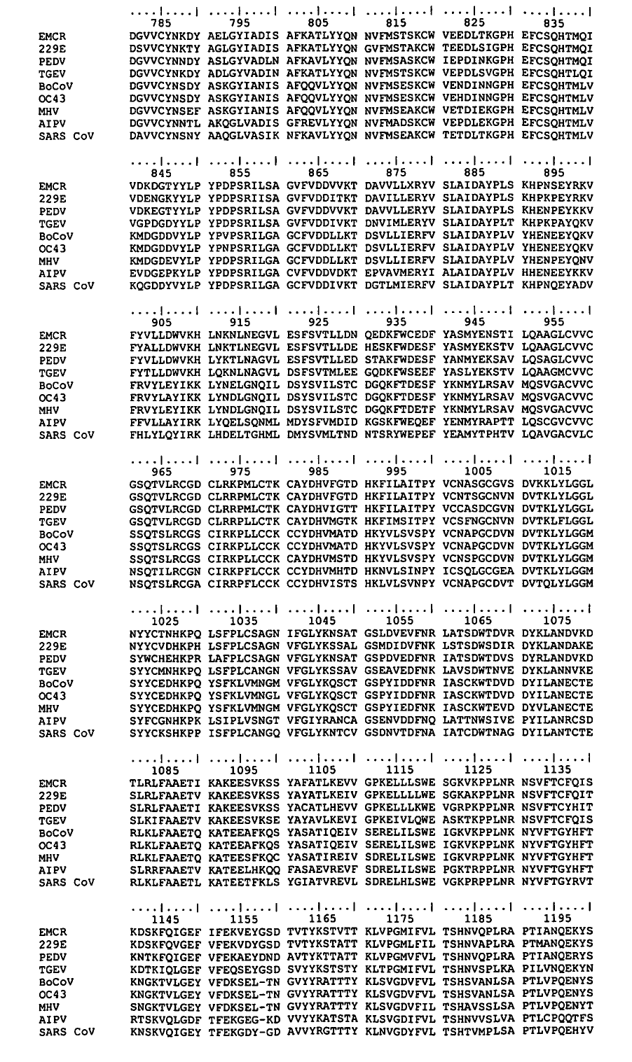

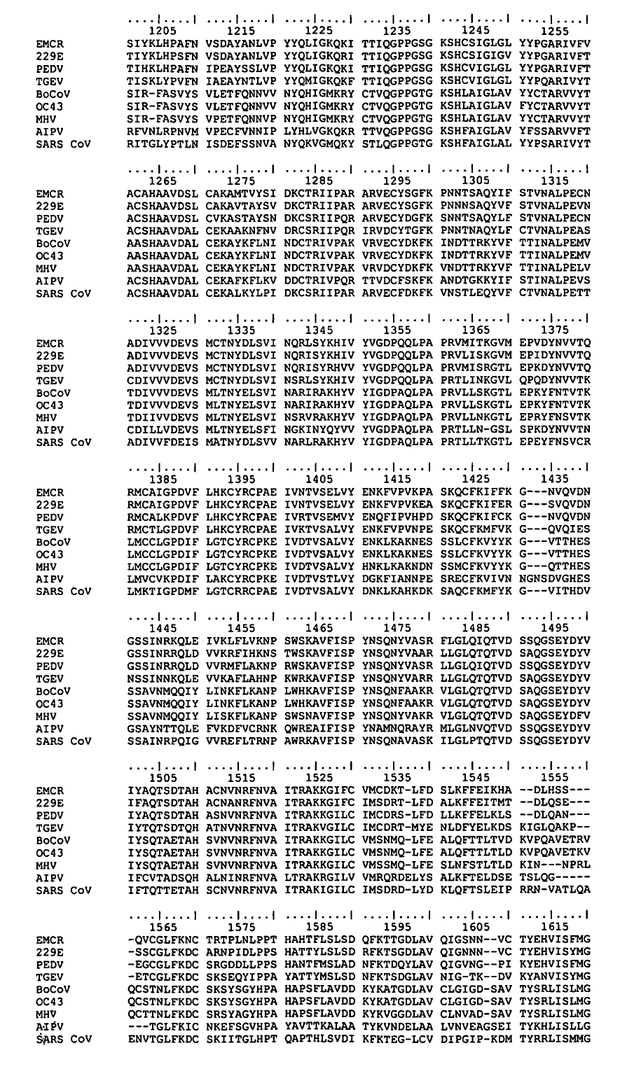

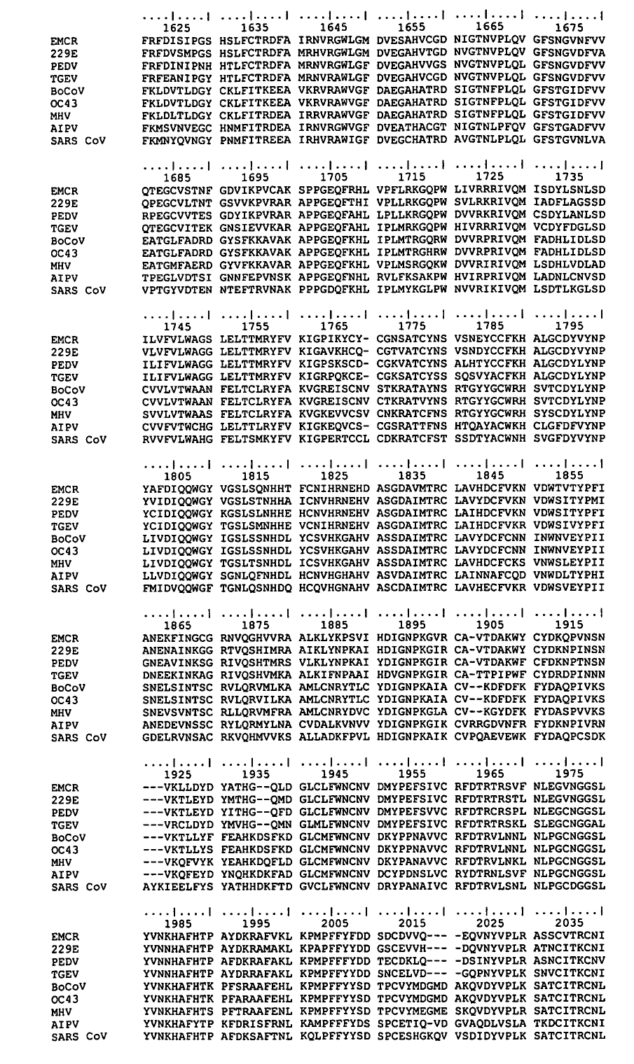

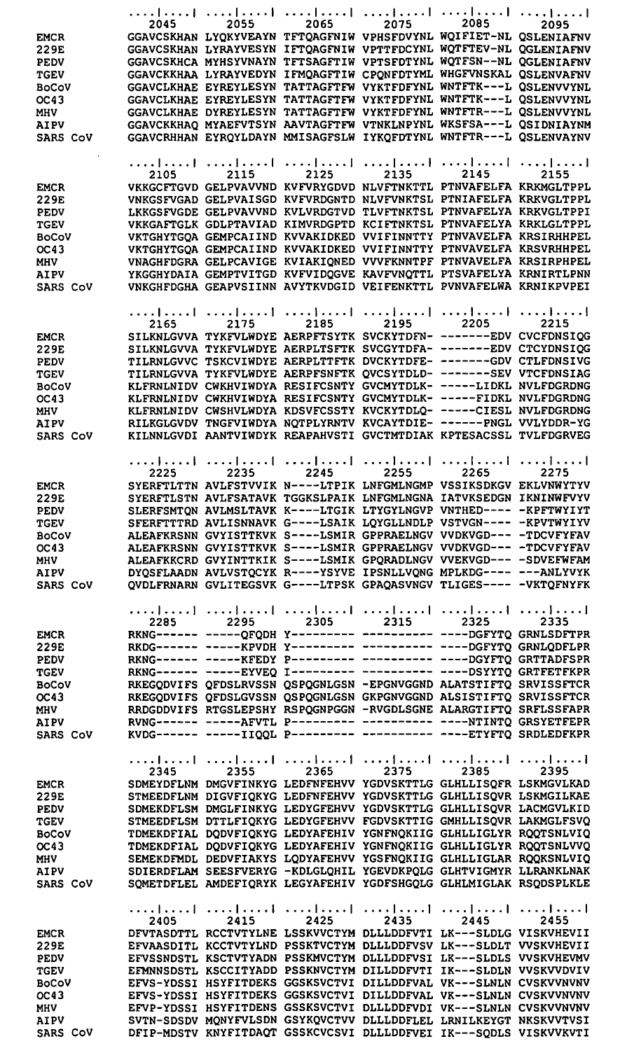

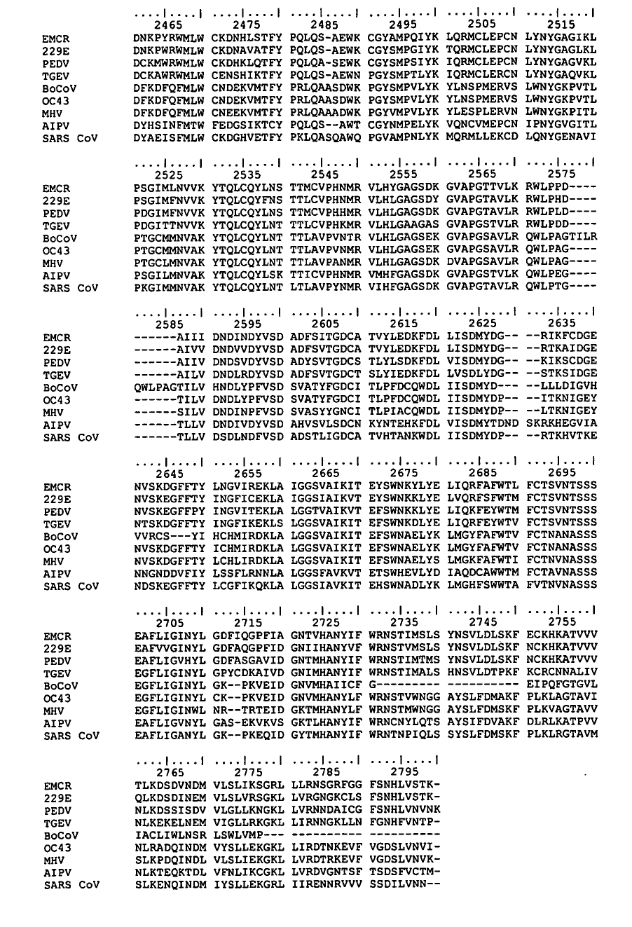

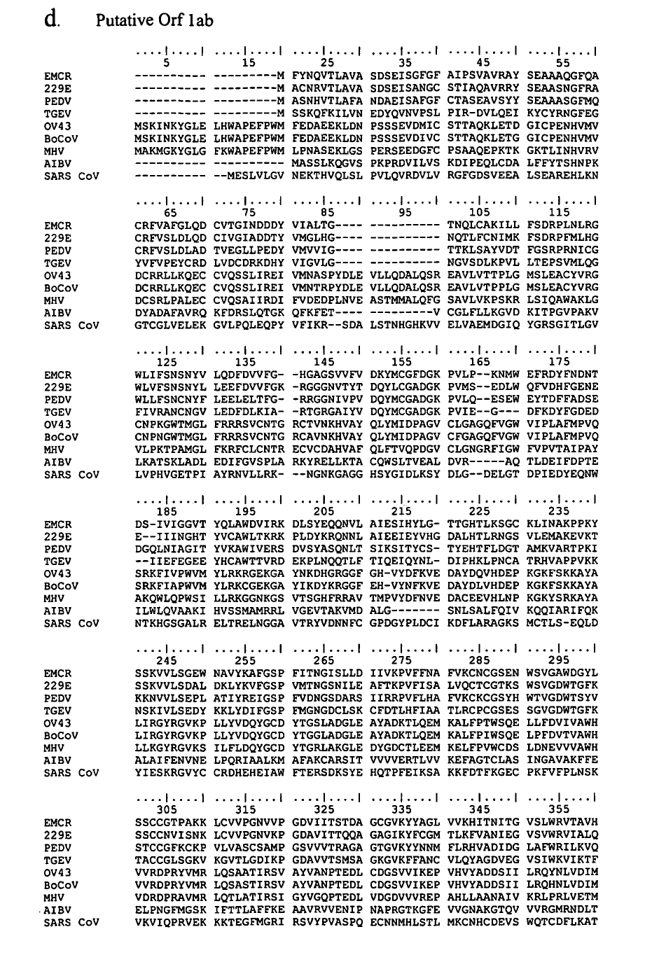

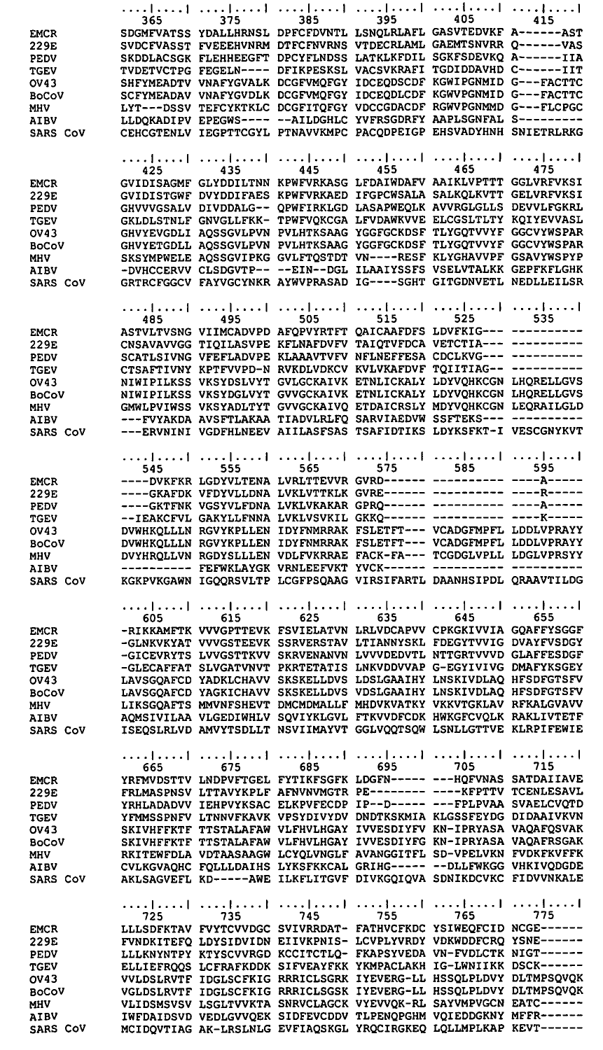

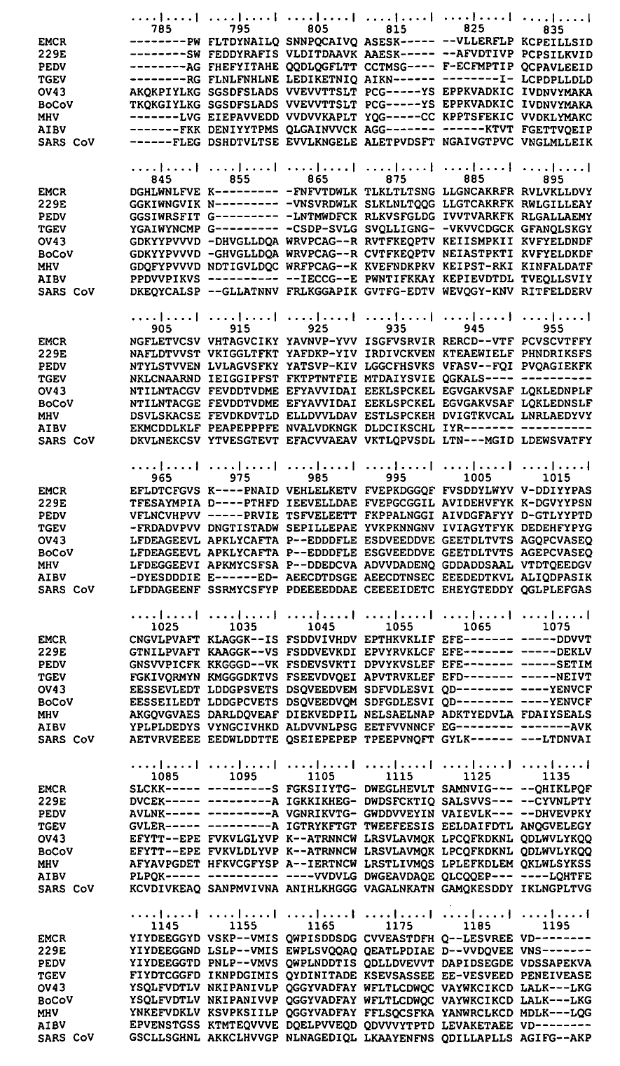

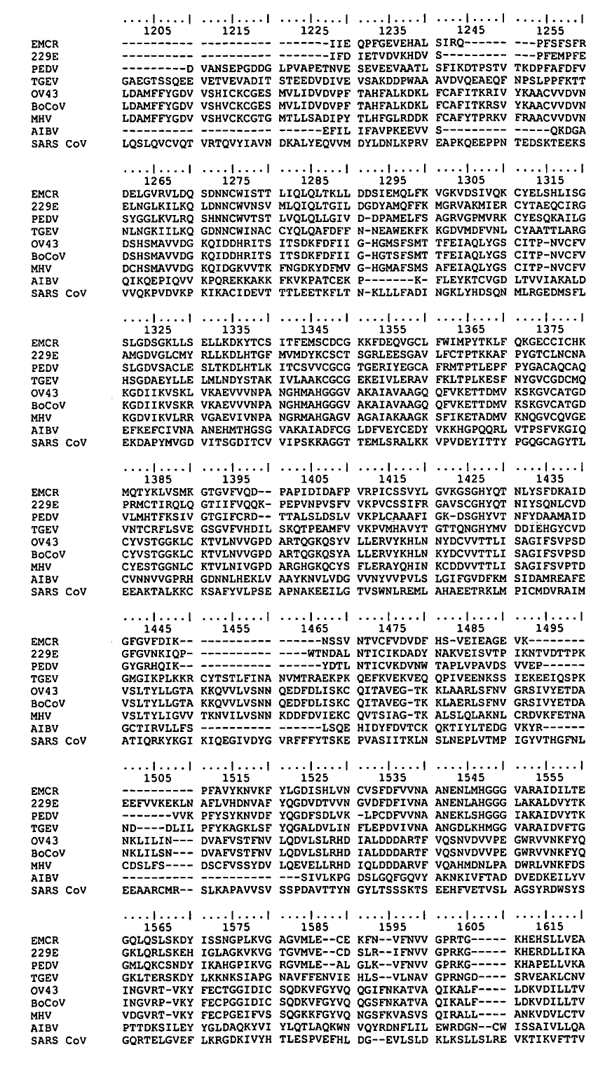

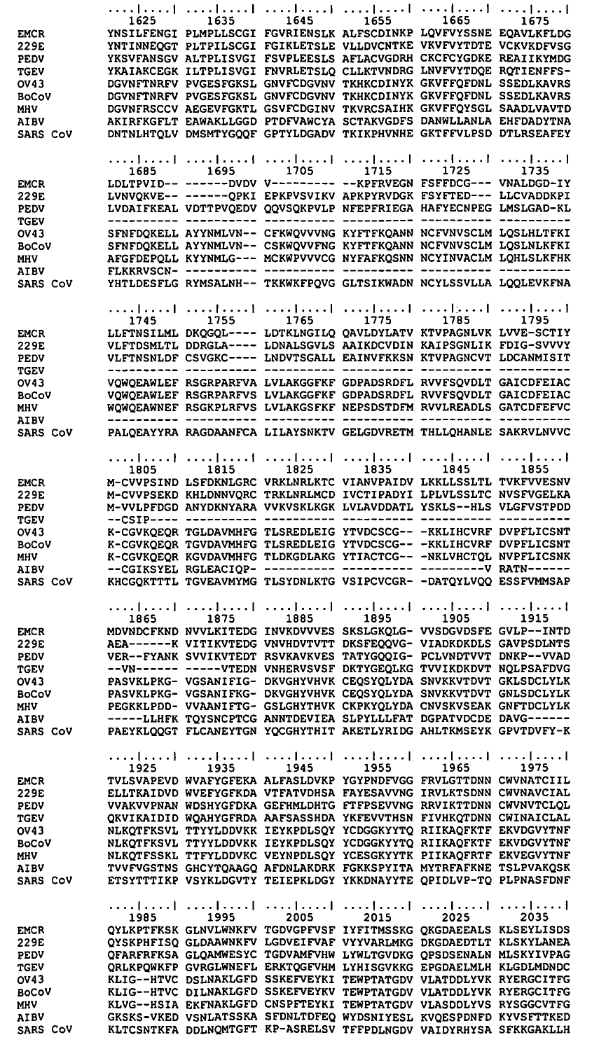

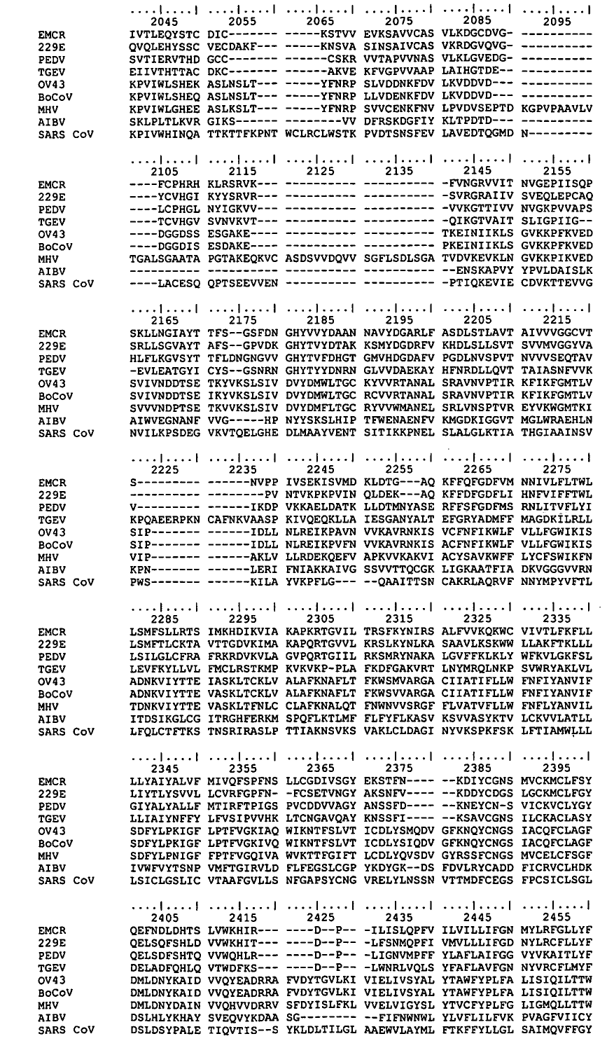

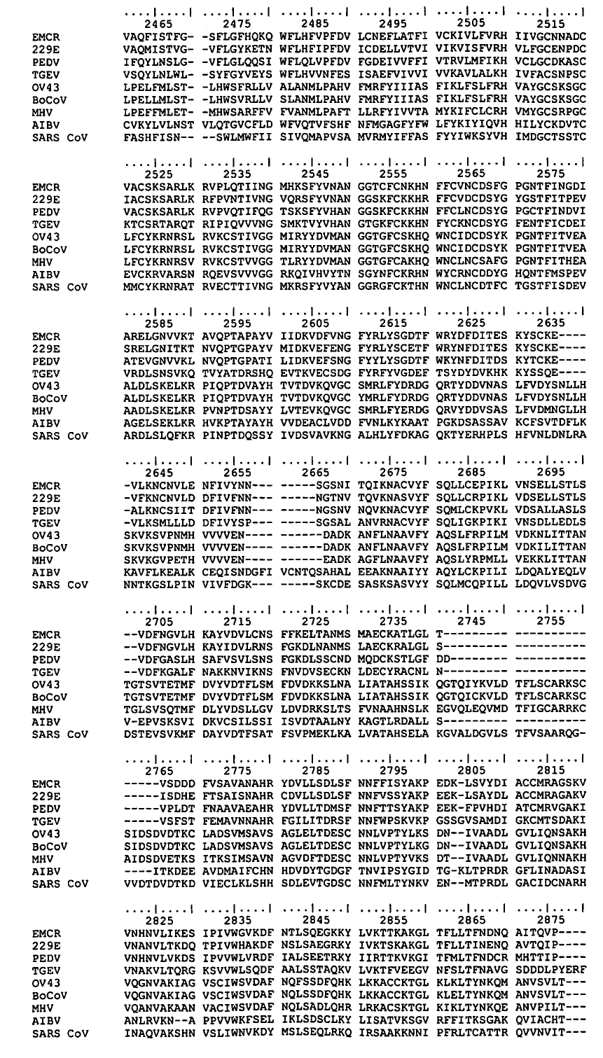

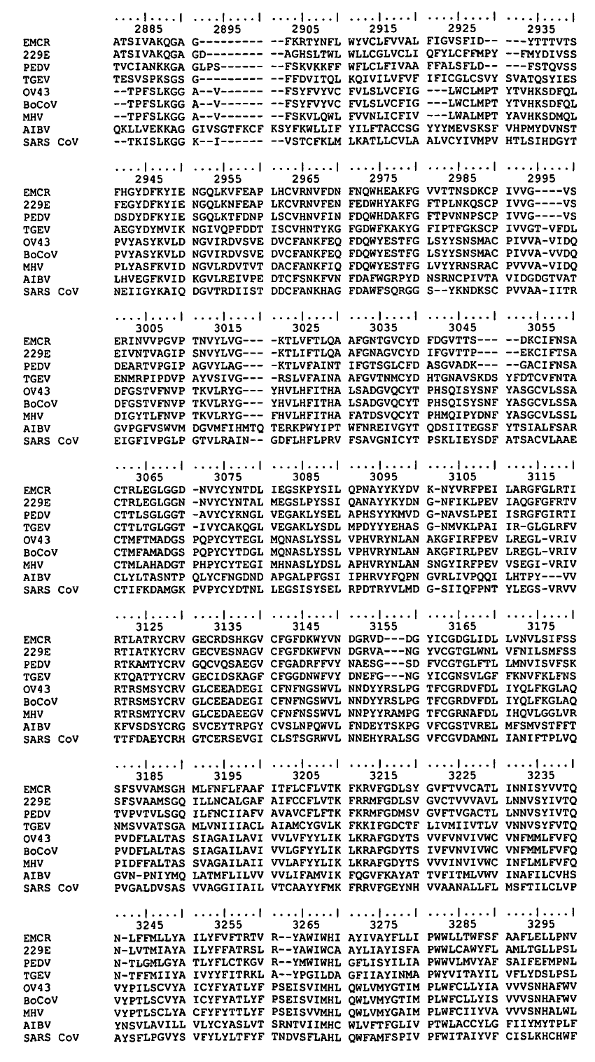

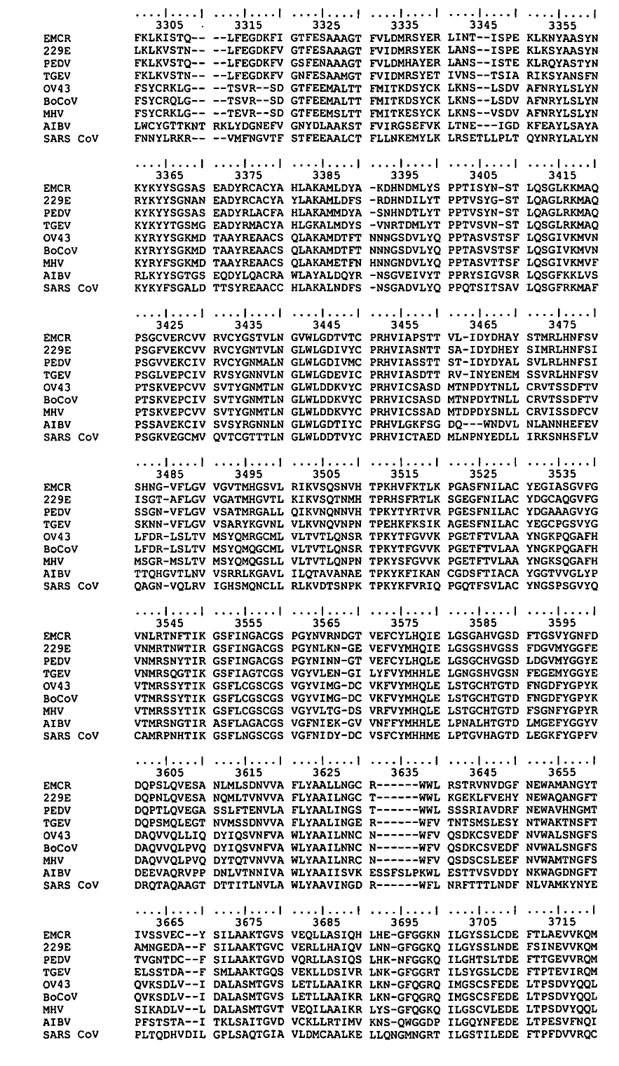

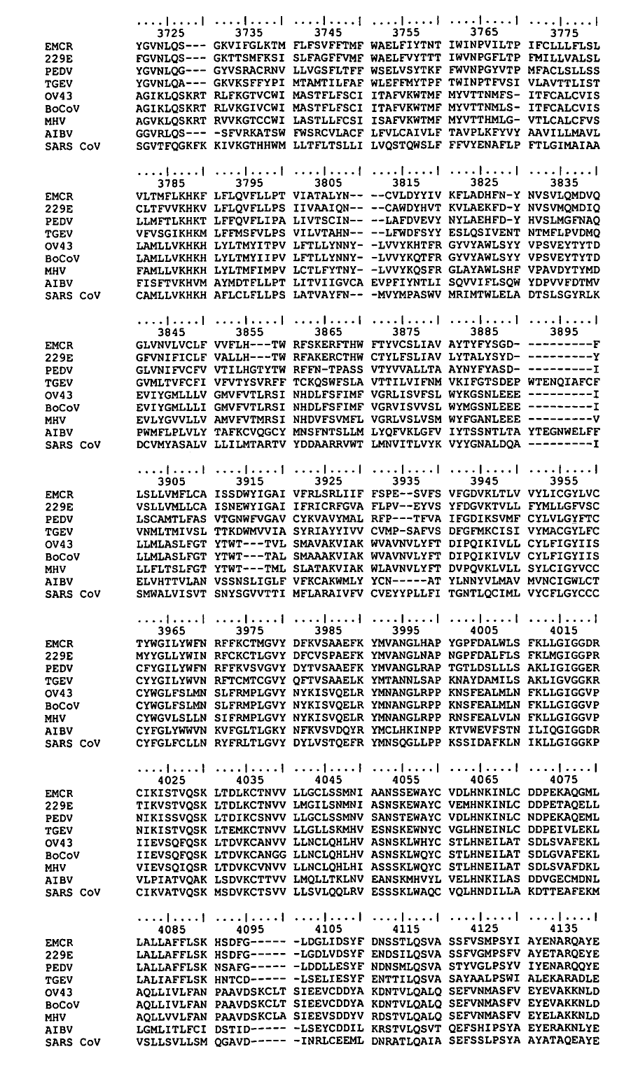

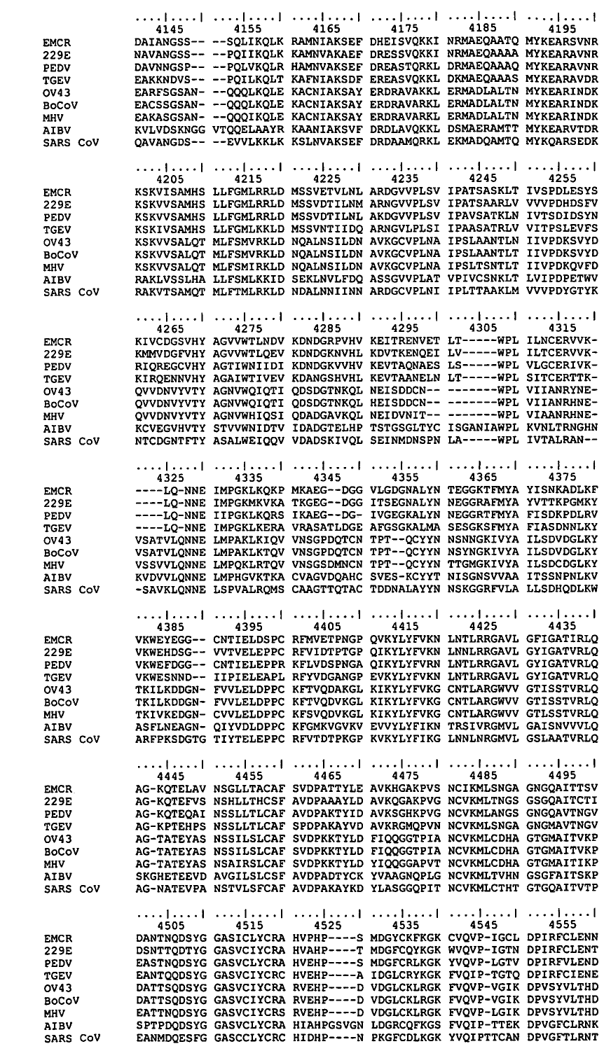

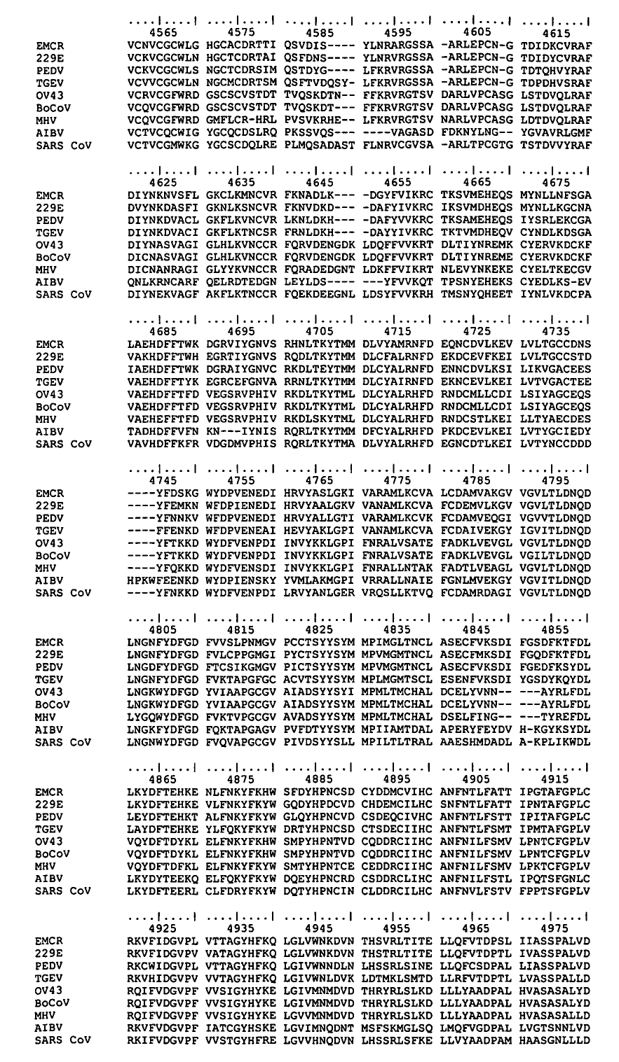

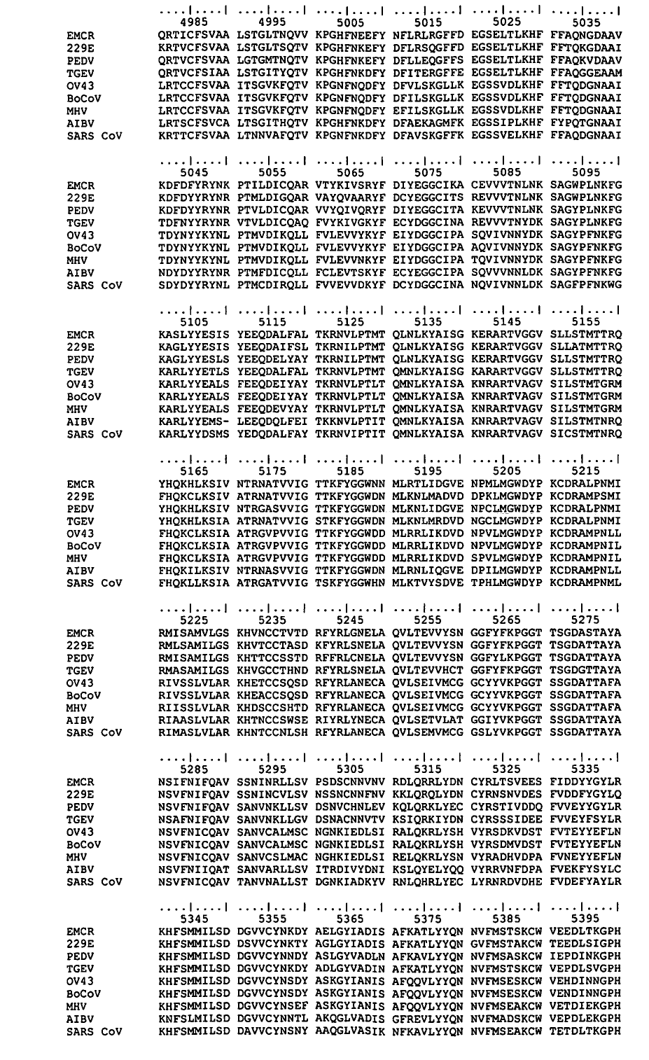

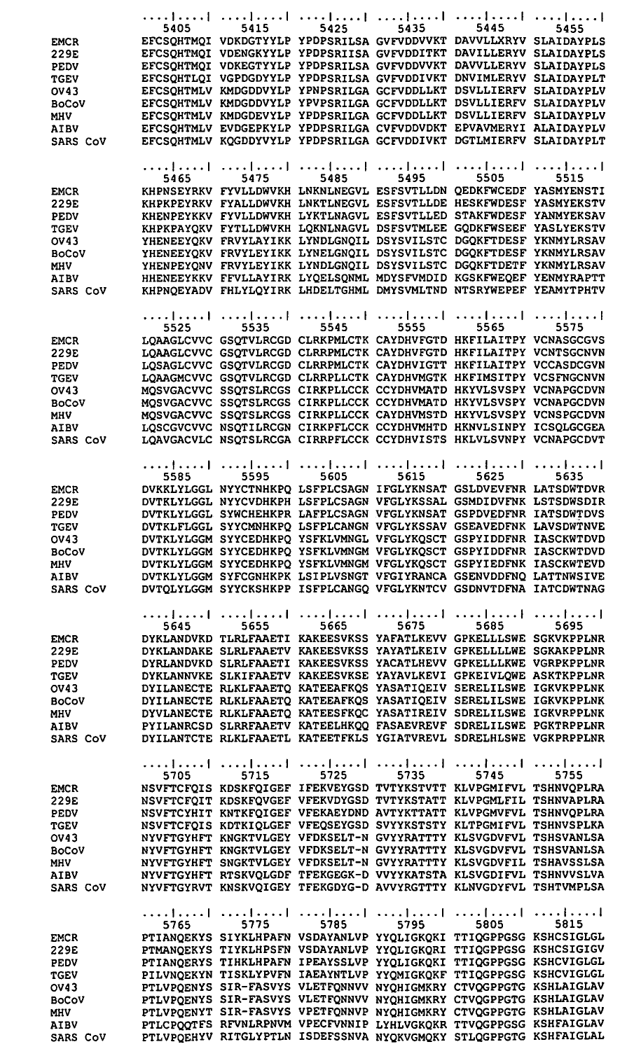

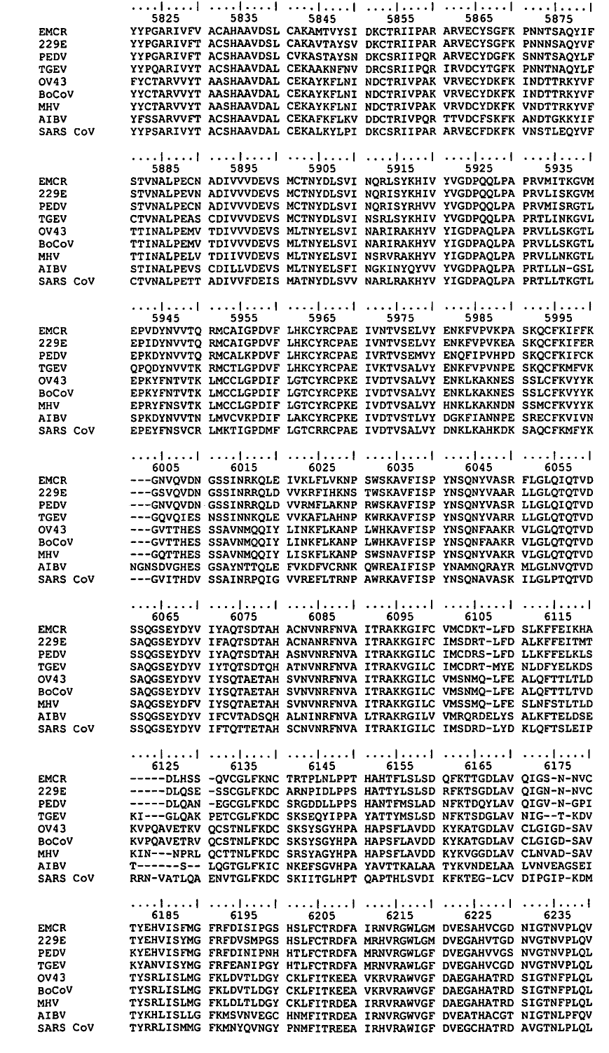

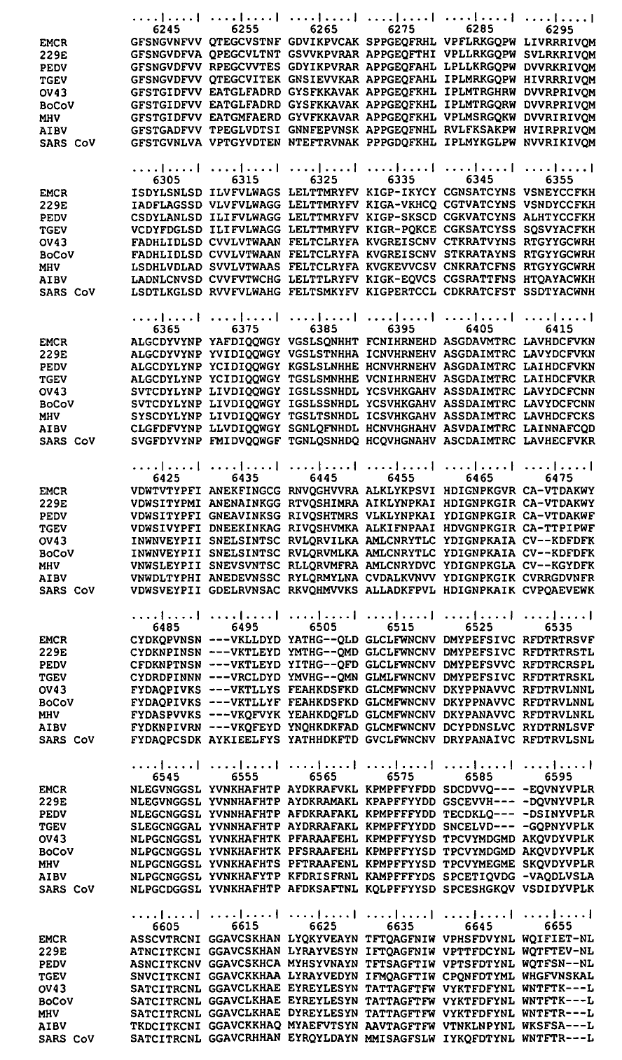

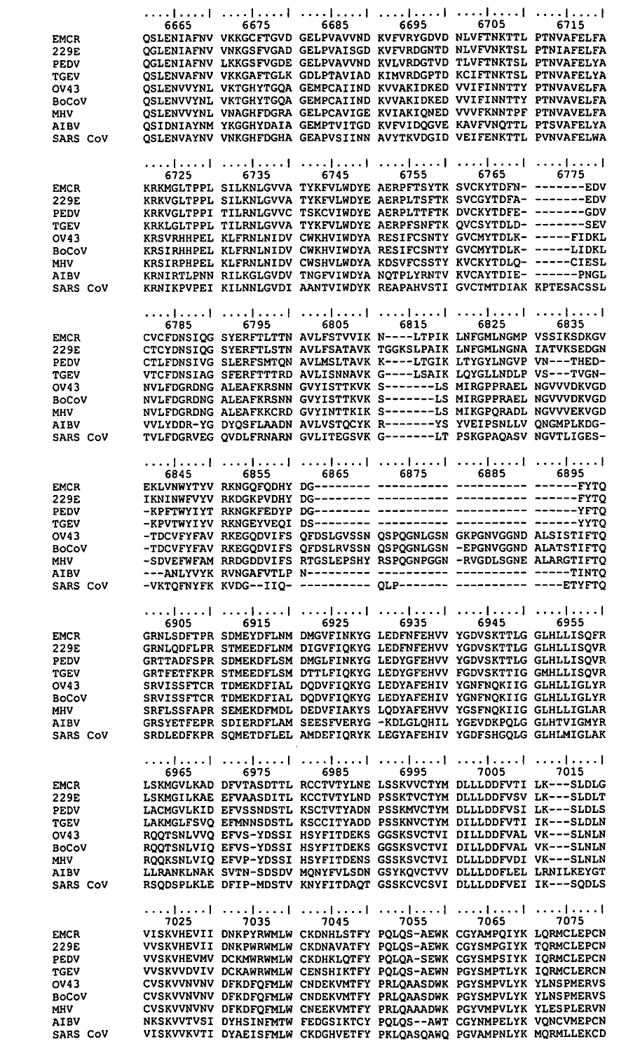

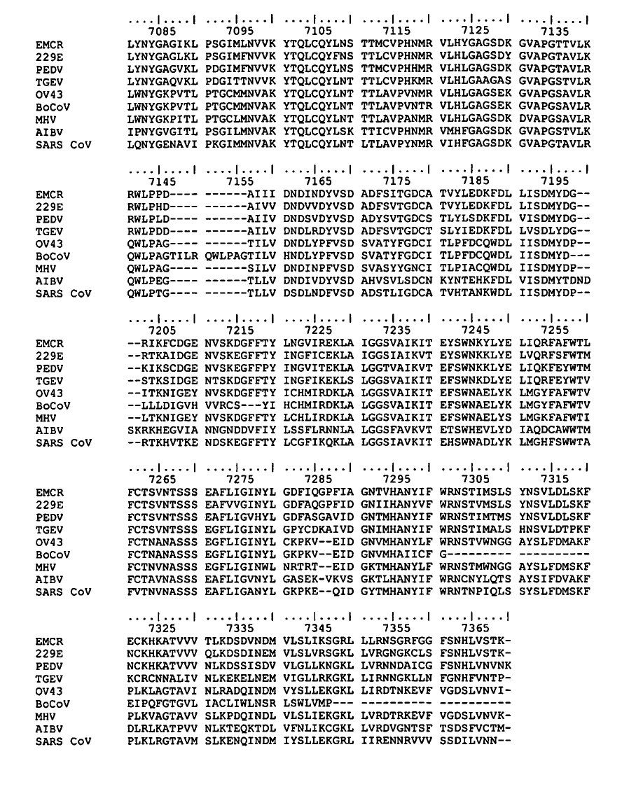

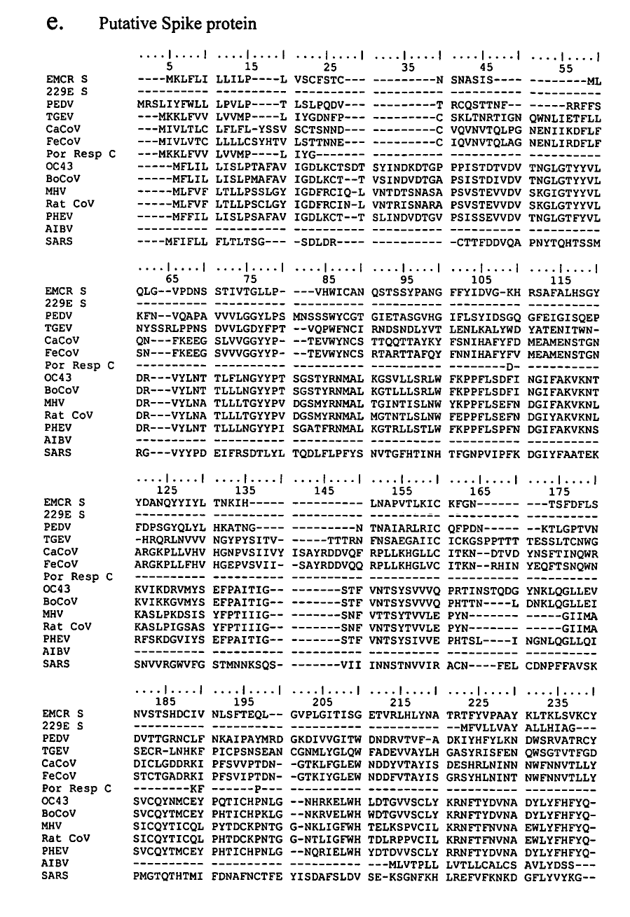

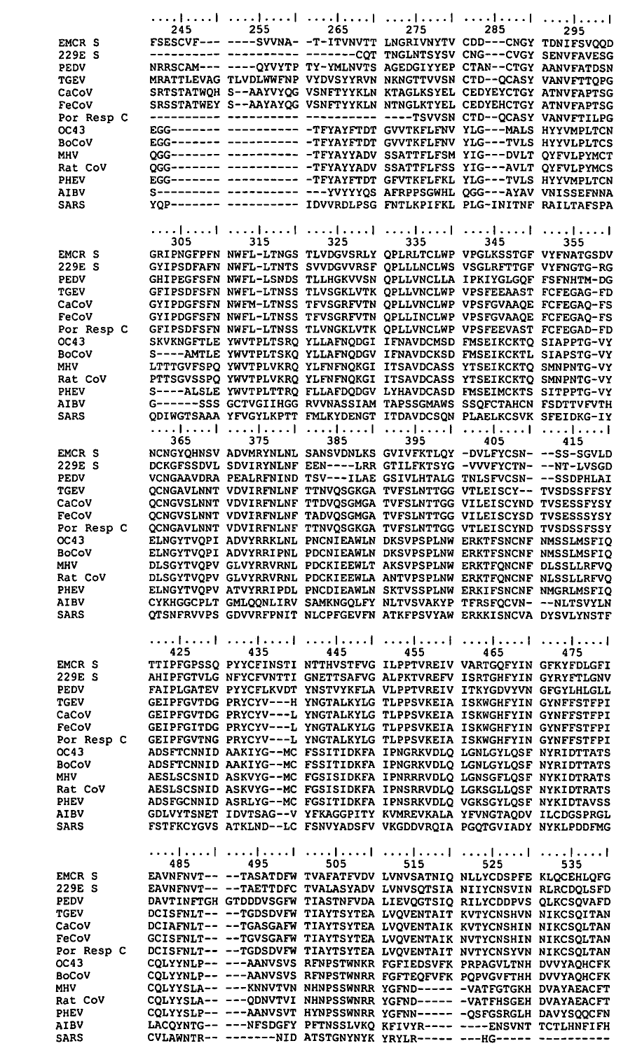

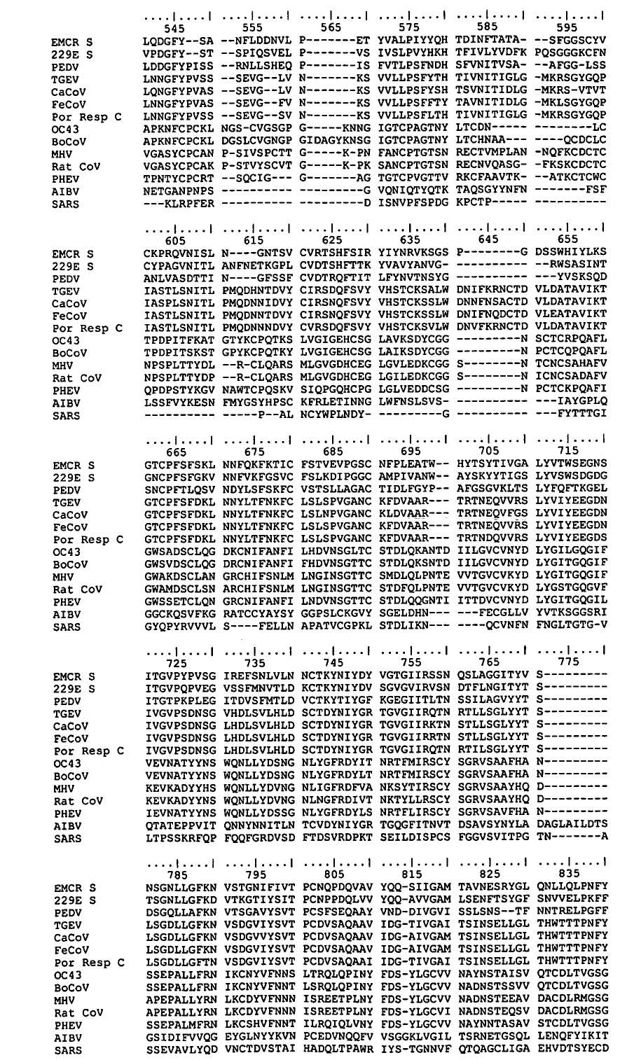

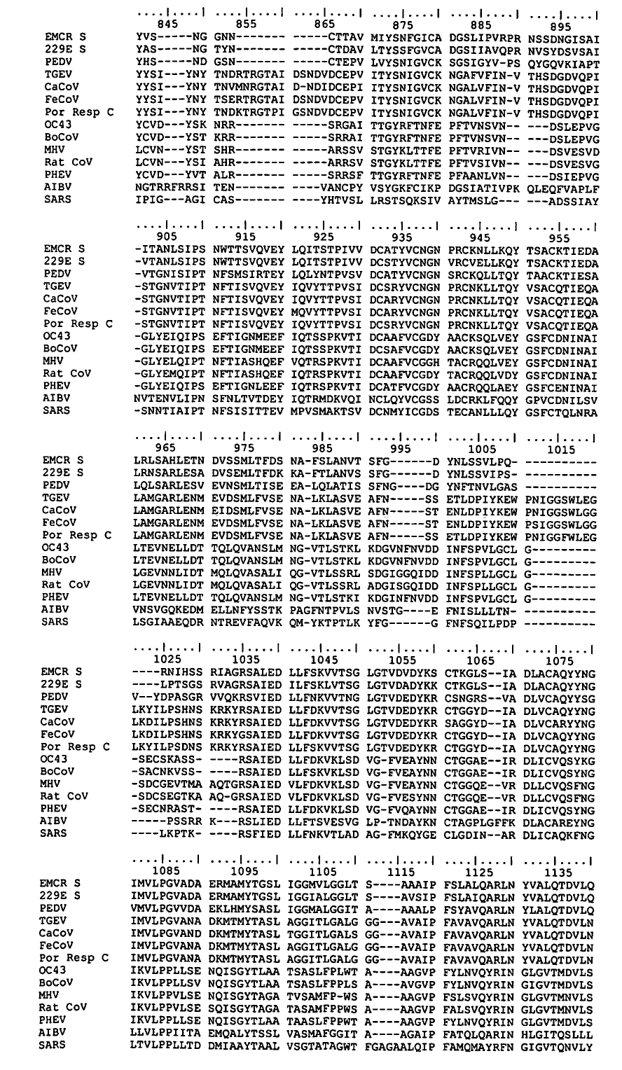

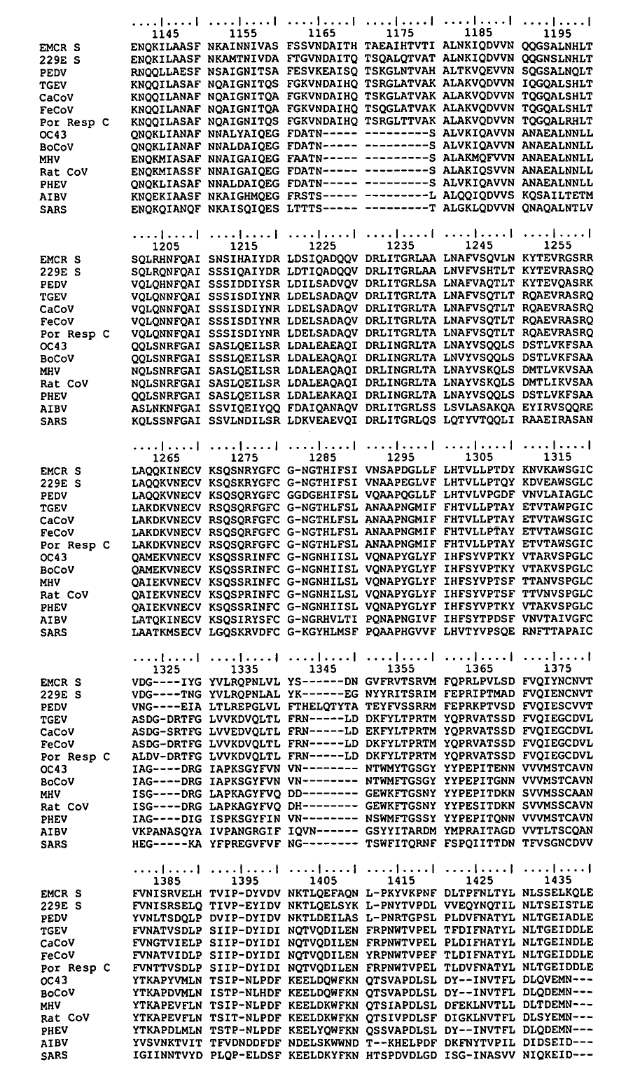

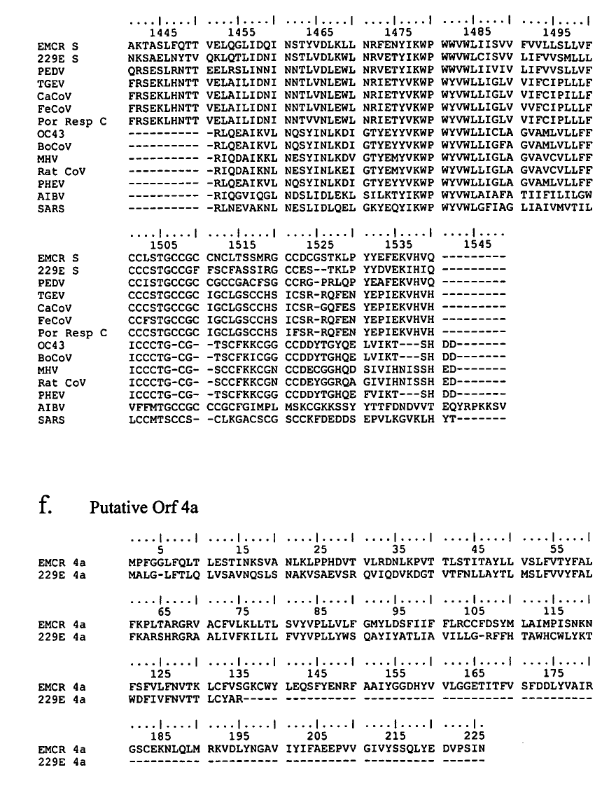

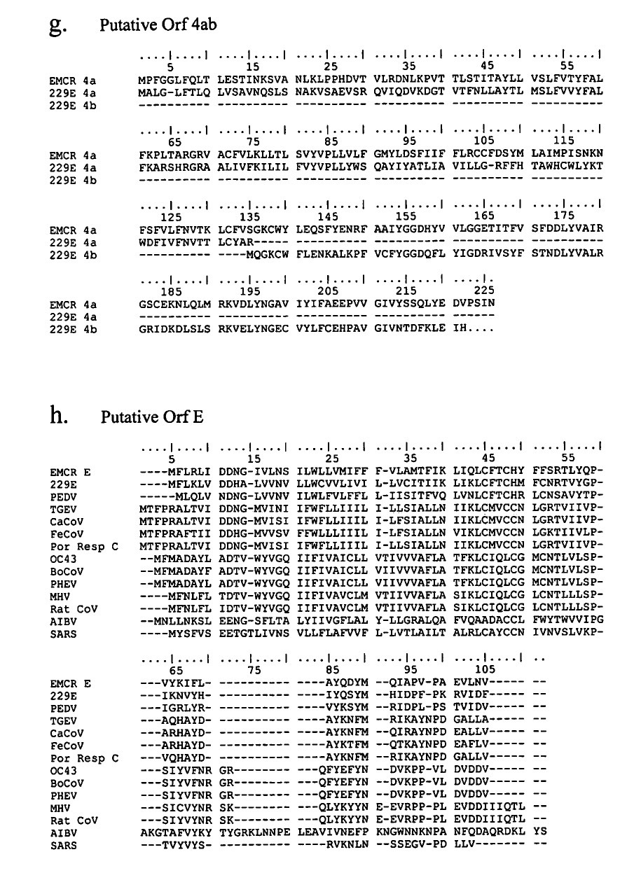

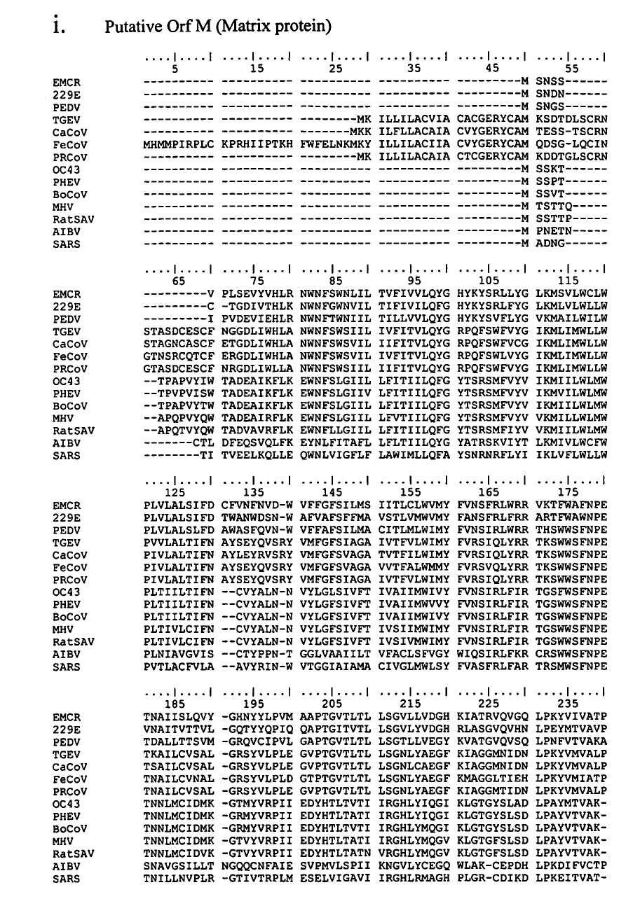

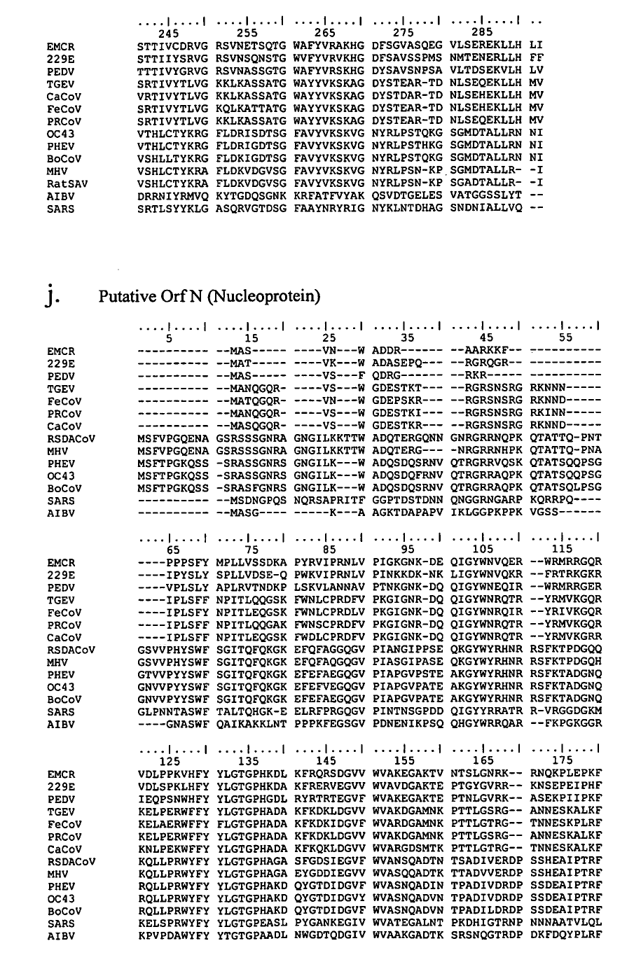

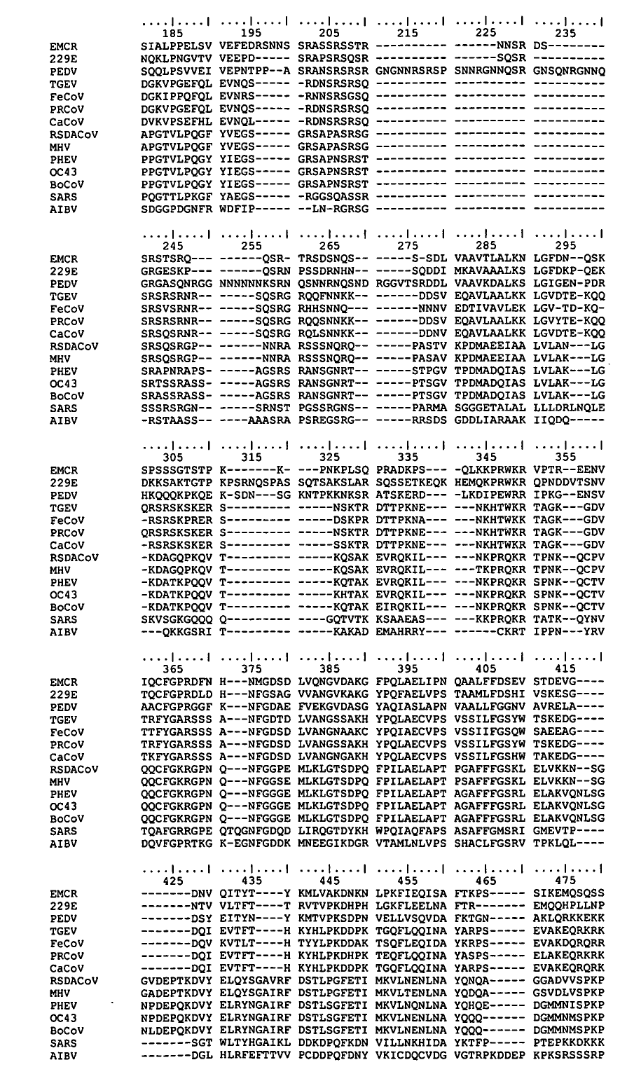

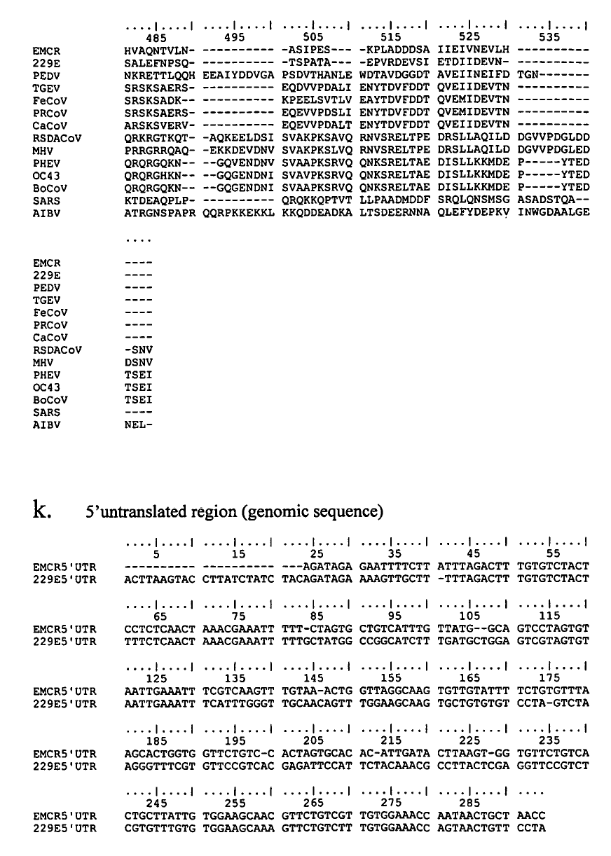

1. An isolated essentially mammalian positive-sense single stranded RNA virus (EMCR-CoV) comprising the sequence of FIG. 1 or homologues thereof.

2. An isolated positive-sense single stranded RNA virus (EMCR-CoV) belonging to the Coronaviruses and identifiable as phylogenetically corresponding thereto by determining a nucleic acid sequence of said virus and testing it in phylogenetic tree analyses wherein maximum likelihood trees are generated using 100 bootstraps and 3 jumbles and finding it to be more closely phylogenetically corresponding to a virus isolate having the sequences as depicted in FIG. 1 than it is corresponding to a virus isolate of PEDV (porcine epidemic diarrhea virus), HCoV-229E (human coronavirus 229E), PRCoV (porcine respiratory coronavirus), TGEV (transmissible gastroenteritis virus), CaCoV (Canine coronavirus) and FeCoV (feline coronavirus).

3. A virus according to claim 1 wherein said nucleic acid sequence comprises an open reading frame (ORF) encoding a viral protein of said virus.

4. A virus according to claim 3 wherein said open reading frame is selected from the group of ORFs encoding the viral replicase, nuclear capsid protein, matrix protein and the spike protein.

5. A virus according to claim 1 isolatable from a human with atypical pneumonia.

6. An isolated or recombinant nucleic acid or EMCR-CoV virus-specific functional fragment thereof obtainable from a virus according to claim 1.

7. A vector comprising a nucleic acid according to claim 6.

8. A host cell comprising a nucleic acid according to claim 6.

9. An isolated or recombinant proteinaceous molecule or EMCR-CoV virus-specific functional fragment thereof encoded by a nucleic acid according to claim 6.

10. An antigen comprising a proteinaceous molecule or EMCR-CoV virus-specific functional fragment thereof according to claim 9.

11. An antibody specifically directed against an antigen according to claim 10.

12. A method for identifying a viral isolate as an EMCR-CoV virus comprising reacting said viral isolate or a component thereof with an antibody according to claim 11.

13. A method for identifying a viral isolate as an EMCR-CoV virus comprising reacting said viral isolate or a component thereof with a nucleic acid according to claim 6.

14. A method for virologically diagnosing an EMCR-CoV infection of a mammal comprising determining in a sample of said mammal the presence of a viral isolate or component thereof by reacting said sample with a nucleic acid according to claim 6.

15. A method for serologically diagnosing an EMCR-CoV infection of a mammal comprising determining in a sample of said mammal the presence of an antibody specifically directed against an EMCR-CoV virus or component thereof by reacting said sample with a proteinaceous molecule or fragment thereof according to claim 9.

16. A diagnostic kit for diagnosing an EMCR-CoV infection comprising a virus according to claim 1.

17. Use of a virus according to claim 1 for the production of a pharmaceutical composition.

18. Use according to claim 17 for the production of a pharmaceutical composition for the treatment or prevention of an EMCR-CoV virus infection.

19. Use according to claim 17 for the production of a pharmaceutical composition for the treatment or prevention of atypical pneumonia.

20. A pharmaceutical composition comprising a virus according to claim 1.

21. A method for the treatment or prevention of an EMCR-CoV virus infection comprising providing an individual with a pharmaceutical composition according to claim 20.

22. A method for the treatment or prevention of atypical pneumonia comprising providing an individual with a pharmaceutical composition according to claim 20.

23. A viral replicase encoded by an RNA sequence comprising the indicated sequences, or homologues thereof as depicted in FIG. 1.

24. A viral spike protein comprising the indicated amino acid sequence as depicted in FIG. 1, or a homologue thereof.

25. A viral nuclear capsid protein encoded by an RNA sequence comprising the indicated sequence as depicted in FIG. 1 or a homologue thereof.

26. A viral nsp 3 or envelope protein encoded by an RNA sequence comprising the indicated sequence as depicted in FIG. 1, or a homologue thereof.

27. A nucleic acid sequence which comprises one or more of the sequences coding for separate viral proteins as depicted in FIG. 1 or a nucleic acid sequence which can hybridise with any of these sequences under stringent conditions.

28. A host cell comprising a vector according to claim 7.

29. A method for virologically diagnosing an EMCR-CoV infection of a mammal comprising determining in a sample of said mammal the presence of a viral isolate or component thereof by reacting said sample with an antibody according to claim 11.

30. A method for serologically diagnosing an EMCR-CoV infection of a mammal comprising determining in a sample of said mammal the presence of an antibody specifically directed against an EMCR-CoV virus or component thereof by reacting said sample with an antigen according to claim 10.

31. A diagnostic kit for diagnosing an EMCR-CoV infection comprising a nucleic acid according to claim 6.

32. A diagnostic kit for diagnosing an EMCR-CoV infection comprising a proteinaceous molecule or fragment thereof according to claim 9.

33. A diagnostic kit for diagnosing an EMCR-CoV infection comprising an antigen according to claim 10.

34. A diagnostic kit for diagnosing an EMCR-CoV infection comprising an antibody according to claim 11.

35. Use of a nucleic acid according to claim 6 for the production of a pharmaceutical composition.

36. Use of a vector according to claim 7 for the production of a pharmaceutical composition.

37. Use of a host cell according to claim 8 for the production of a pharmaceutical composition.

38. Use of a proteinaceous molecule or fragment thereof according to claim 9 for the production of a pharmaceutical composition.

39. Use of an antigen according to claim 10 for the production of a pharmaceutical composition.

40. Use of an antibody according to claim 11 for the production of a pharmaceutical composition.

41. A pharmaceutical composition comprising a nucleic acid according to claim 6.

42. A pharmaceutical composition comprising a vector according to claim 7.

43. A pharmaceutical composition comprising a host cell according to claim 8.

44. A pharmaceutical composition comprising a proteinaceous molecule or fragment thereof according to claim 9.

45. A pharmaceutical composition comprising an antigen according to claim 10.

46. A pharmaceutical composition comprising an antibody according to claim 11.
